(19) United States
(12) Patent Application Publication
(10) Pub. No.: US 2024/0230630 A1
(43) Pub. Date: Jul. 11, 2024

(54) USES AND METHODS FOR ELEMENTOMIC CHARACTERIZATION ANALYSIS IN DIAGNOSIS AND PROGNOSIS OF MEDICAL DISEASES AND CONDITIONS

(71) Applicant: SHANGHAITECH UNIVERSITY, Pudong District, Shanghai (CN)

(72) Inventors: Waichi SHUM, Shanghai (CN); Baoli ZHANG, Shanghai (CN); Huijuan SHI, Shanghai (CN)

(73) Assignee: SHANGHAITECH UNIVERSITY, Pudong District, Shanghai (CN)

(21) Appl. No.: 18/559,264

(22) PCT Filed: Apr. 13, 2022
(86) PCT No.: PCT/CN2022/086488

§ 371 Date: Nov. 6, 2023

(30) Foreign Application Priority Data
May 7, 2021 (CN) 202110493989.4

(51) Int. Cl. G01N 33/50; H01J 49/10

(52) U.S. Cl. CPC G01N 33/5091; H01J 49/105; G01N 2800/367

(57) ABSTRACT

The present application provides a method for detecting infertile spermatozoa in a sample, the method comprising: detecting a concentration of at least one metal in the sample that falls outside of a predetermined range using single cell inductively coupled plasma mass spectrometry (sc-ICP-MS). It also provides a method for detecting infertile spermatozoa in a sample, which comprising: detecting a dynamic or kinetic parameter of a signal spike of at least one metal selected from the group of several metallic elements by sc-ICP-MS.

### CROSS-REFERENCE TO RELATED APPLICATIONS

This application claims the benefit of and priority to Chinese Patent Application No. 202110493989.4, filed May 7, 2021, the entire contents of which are incorporated herein by reference in its entirety.

### FIELD

The present technology relates to the fields of medical diagnosis, patient monitoring, and treatment efficacy evaluation. It is directed to methods, applications and kits related to detecting (i) a concentration, or (ii) a dynamic or kinetic parameter of a signal spike, or both (i) and (ii), of at least one metal in spermatozoa using single-cell inductively coupled plasma mass spectrometry (sc-ICP-MS). The technology is suited to detect fertile or infertile spermatozoa in a sample.

### BACKGROUND

The following description is provided to assist the understanding of the reader. None of the information provided or references cited is admitted to be prior art.

Infertility has become a prevalent health issue and has been identified by the World Health Organization (WHO) as a disease in 2009 (see, for example, Zegers-Hochschild et al., Fertil Steril, 2009, 92, 1520-1524), only the third to cancer and cardiovascular diseases. Currently, about 15% of couples at their reproductive age worldwide suffer from infertility (see, for example, Levine et al., Hum Reprod Update, 2017, 23, 646-659; and Joffe, Human Reproduction, 2010, 25, 295-307). Male factors account for about 50% of all infertility cases and in some clinics can be even mounted up to 70% of male evaluations (see, for example, Agarwal et al., Reprod Biol Endocrinol, 2015, 13, 37; and Tuttelmann et al., Med Genet, 2018, 30, 12-20).

The main factors causing male infertility are sperm functional defects, sperm deficiency, infection, sexual dysfunction, and disorders in endocrine and genetics (Krausz and Riera-Escamilla, 2018, Nat Rev Urol 15, 369-384; Mehra et al., 2018, Urologia 85, 22-24; Punab et al., 2017, Hum Reprod 32, 18-31; Vander Borght and Wyns, 2018, Clin Biochem 62, 2-10; Wall and Jayasena, 2018, BMJ 363, k3202; and Zhou et al., 2018, BJOG 125, 432-441). Among these factors, azoospermia, asthenozoospermia, sexual dysfunction and other symptoms related to sperm physical parameters are easier to diagnose by conventional means (Centola, 2014, Urol Clin NAm 41, 163-7; Krausz and Riera-Escamilla; Punab et al.; Wall and Jayasena; and WHO, WHO laboratory manual for the examination and processing of human semen, World Health Organization, Geneva, Switzerland, 6th edn., 2021). Currently, clinical analyses of male infertility rely mainly on semen analysis and physical-based sperm parameters and the traditional methods of male infertility diagnosis mainly focus on the morphological observation of sperm and the biochemical component analysis of seminal plasma, such as sperm morphology and motility, semen appearance, immunology evaluation, seminal plasma biochemical detection and karyotype parameters, according to the 6^{th} edition of WHO Laboratory Manual for Examination and Processing of Human Semen (see, WHO, 2021). However, there are still about 30% of infertile men diagnosed with normal semen analysis, and the diagnostic failure rate is still ranging 30% to 70% of clinical cases, and most of the cases, which is 72% of male infertility cases in some developed countries, are still labeled idiopathic (see, for example, Agarwal et al., 2021, Lancet 397, 319-333; Hamada et al., 2012, Int Braz J Urol 38, 576-594; Mascarenhas et al., 2012. Plos Med 9; and Tuttelmann et al.).

A new technology is therefore urgently needed to assess the functional quality of human sperm. This disclosure satisfies this need and provides related advantages as well.

### SUMMARY OF THE PRESENT DISCLOSURE

In one aspect, the present disclosure provides a method for detecting infertile spermatozoa in a sample obtained from a subject.

In some embodiments, the method comprises, or consists essentially of, or yet further consists of detecting a concentration of at least one metal in the sample that falls outside of a predetermined range using single cell inductively coupled plasma mass spectrometry (sc-ICP-MS).

In some embodiments, wherein the at least one metal is selected from the group consisting of sodium (Na), potassium (K), calcium (Ca), magnesium (Mg), zinc (Zn), iron (Fe), copper (Cu), selenium (Se), cobalt (Co), chromium (Cr), cadmium (Cd), manganese (Mn), arsenic (As), mercury (Hg), lead (Pb), silver (Ag), aluminium (Al), and nickel (Ni).

In some embodiments, the sample is diluted with a buffer prior to the detecting step.

In some embodiments, the predetermined range corresponds to metal concentrations detected in spermatozoa from a population of fertile subjects.

In some embodiments, the spermatozoa are capacitated.

In some embodiments, the spermatozoa are not capacitated.

In some embodiments, the predetermined range of the Na concentration is between about 5 attogram (ag) to about 50,000 ag for spermatozoa that are not capacitated and between about 25 ag to about 50,000 ag for spermatozoa that are capacitated; wherein the predetermined range of the K concentration is between about 50 ag to about 50,000 ag for spermatozoa that are not capacitated and between about 280 ag to about 50,000 ag for spermatozoa that are capacitated; wherein the predetermined range of the Ca concentration is between about 200 ag to about 50,000 ag for spermatozoa that are not capacitated and between about 700 ag to about 20,500 ag for spermatozoa that are capacitated; wherein the predetermined range of the Mg concentration is between about 8 ag to about 50000 ag for spermatozoa that are not capacitated and between about 75 ag to about 15,100 ag for spermatozoa that are capacitated; wherein the predetermined range of the Zn concentration is between about 5 ag to about 50,000 ag for spermatozoa that are not capacitated and between about 20 ag to about 50,000 ag for spermatozoa that are capacitated; wherein the predetermined range of the Fe concentration is between about 5 ag to about 50,000 ag for spermatozoa that are not capacitated and between about 13 ag to about 50,000 ag for spermatozoa that are capacitated; wherein the predetermined range of the Al concentration is between about 3 ag to about 50,000 ag for spermatozoa that are not capacitated and between about 6 ag to about 50000 ag (e.g., such as between about 6 ag and about 46700 ag) for spermatozoa that are capacitated; wherein the predetermined range of the Se concentration is between about 59 ag to about 50,000 ag for spermatozoa that are not capacitated and between about 62 ag to about 45,810 ag for spermatozoa that are capacitated; wherein the predetermined range of the Co concentration is between about 3 ag to about 3,700 ag for spermatozoa that are not capacitated and between about 9 ag to about 20,200 ag for spermatozoa that are capacitated; wherein the predetermined range of the Cu concentration is between about 9 ng to about 50,000 ag for spermatozoa that are not capacitated and between about 9 ng to about 37,590 ag for spermatozoa that are capacitated; wherein the predetermined range of the Cr concentration is between about 4 ng to about 50,000 ag for spermatozoa that are not capacitated and between about 5 ag to about 46,000 ag for spermatozoa that are capacitated; and wherein the predetermined range of the Mn concentration is between about 2 ag to about 50,000 ag for spermatozoa that are not capacitated and between about 7 ag to about 32,610 ag for spermatozoa that are capacitated.

In one aspect, the present disclosure provides a method for detecting infertile spermatozoa in a sample obtained from a subject, comprising: detecting a dynamic or kinetic parameter of a signal spike of at least one metal selected from the group of sodium (Na), potassium (K), calcium (Ca), magnesium (Mg), zinc (Zn), iron (Fe), copper (Cu), selenium (Se), cobalt (Co), chromium (Cr), cadmium (Cd), manganese (Mn), arsenic (As), mercury (Hg), lead (Pb), silver (Ag), aluminium (Al), and nickel (Ni) in the sample that falls outside of a predetermined range by single cell inductively coupled plasma mass spectrometry (sc-ICP-MS)

In some embodiments, the predetermined range corresponds to dynamic or kinetic parameters detected in spermatozoa from a population of fertile subjects.

In some embodiments, the dynamic or kinetic parameter of the spike are selected from: dwell time, pre-peak dwell time, post-peak dwell time, peak time, ratio between the peak time and the dwell time, raising tau constant before the peak, dynamic area before the peak, tailing tau constant after the peak, dynamic area after the peak, or any combination thereof.

In some embodiments, the dynamic or kinetic parameter of the spike comprises: (a) a dwell time of the Fe spike between about 1.4 to about 7.9 ms for spermatozoa that are not capacitated and a dwell time of the Fe spike between about 1.5 to about 6.7 ms for spermatozoa that are capacitated; (b) a tailing tau constant after the peak of the Fe spike about between about 0.18 to about 0.81 ms for spermatozoa that are not capacitated and a tailing tau constant after the peak of the Fe spike between about 0.18 to about 0.90 ms for spermatozoa that are capacitated; (c) a raising tau constant before the peak of the Fe spike at about −0.35 ms or lower for spermatozoa that are not capacitated and a raising tau constant before the peak of the Fe spike at about −0.80 ms for spermatozoa that are capacitated; (d) a dwell time of the Cu spike at about 1.5 ms or shorter for spermatozoa that are not capacitated and that are capacitated; (e) a raising tau constant before the peak of the Cu spike at about −0.2 ms or lower for spermatozoa that are not capacitated and a raising tau constant before the peak of the Cu spike at about −0.6 ms or lower for spermatozoa that are capacitated; (f) a tailing tau constant after the peak of the Cu spike at about 0.15 ms or lower for spermatozoa that are not capacitated and a tailing tau constant after the peak of the Cu spike at about 0.2 ms or lower for spermatozoa that are capacitated; (g) a dwell time of the Zn spike at about 2.1 ms or shorter for spermatozoa that are not capacitated and a dwell time of the Zn spike at about 1.2 ms or shorter for spermatozoa that are capacitated; (h) a raising tau constant before the peak of the Zn spike at about −0.25 ms or lower for spermatozoa that are not capacitated and a raising tau constant before the peak of the Zn spike at about −0.20 ms or lower for spermatozoa that are capacitated; (i) a tailing tau constant after the peak of the Zn spike at about 1.15 ms or lower for spermatozoa that are not capacitated and a tailing tau constant after the peak of the Zn spike at about 0.25 ms or lower for, spermatozoa that are capacitated; (j) a dwell time of the Cr spike at about 3.25 ms or shorter for spermatozoa that are not capacitated and a raising tau constant before the peak of the Cr spike at about −0.45 ms or lower for spermatozoa that are not capacitated; (k) a tailing tau constant after the peak of the Cr spike between about 0.2 to about 0.5 ms for spermatozoa that are not capacitated; (1) a dwell time of the Se spike at about 1.5 ms or shorter for spermatozoa that are not capacitated; (m) a raising tau constant before the peak of the Se spike at about −0.15 ms or lower for spermatozoa that are not capacitated; and (n) a tailing tau constant after the peak of the Se spike at about 0.25 ms or lower for spermatozoa that are not capacitated.

In an aspect, the present disclosure provides a method for detecting infertile spermatozoa in a sample, comprising (i) contacting a first population of the spermatozoa from the subject with a Human tubal fluid (HTF) buffer and (ii) detecting a concentration of at least one metal selected from the group of potassium (K), calcium (Ca), magnesium (Mg), mercury (Hg), silver (Ag), and aluminum (Al) in the first population of spermatozoa post the contacting step that is comparable to or lower than concentration present in a second population of spermatozoa not contacted with a HTF buffer using single-cell inductively coupled plasma mass spectrometry (sc-ICP-MS); or (iii) detecting a concentration of at least one metal selected from the group of sodium (Na), potassium (K), calcium (Ca), magnesium (Mg), zinc (Zn), iron (Fe), copper (Cu), selenium (Se), cobalt (Co), chromium (Cr), cadmium (Cd), manganese (Mn), arsenic (As), mercury (Hg), lead (Pb), silver (Ag), aluminium (Al), and nickel (Ni) in the first population of spermatozoa post the contacting step that is comparable to or lower than a concentration present in a second population of spermatozoa not contacted with a HTF buffer using single-cell inductively coupled plasma mass spectrometry (sc-ICP-MS); or (iv) detecting a concentration of selenium (Se) in the first population of spermatozoa post the contacting step that is comparable to or higher than a concentration present in the second population of spermatozoa not contacted with a HTF buffer using the sc-ICP-MS; or (v) both (ii), (iii) and (iv).

In some embodiments, the sample is obtained from a subject.

In some embodiments, the subject has or suspect of having idiopathic infertility, asthenozoospermia, oligozoospermia, or oligoasthenozoospermia.

In some embodiments, the method further comprising treating the subject with an infertility therapy or infertility procedure.

In some embodiments, the concentration of at least one metal in the spermatozoa is lower than the predetermined range and the infertility therapy comprises treatment with the at least one metal.

In some embodiments, two or more of the metals are detected.

In some embodiments, the spermatozoa is diluted to a concentration of 3×10⁶ spermatozoa/ml or less prior to the detecting step.

In some embodiments, wherein the spermatozoa is diluted to 10 times or more prior to the detecting step.

In some embodiments, the spermatozoa is centrifuged to remove the seminal plasma prior to the dilution.

In some embodiments, the sample comprises semen, optionally liquefied; or fixed; or capacitated; or cryopreserved; or liquefied and fixed; or liquefied and capacitated; or liquefied and cryopreserved; or fixed and capacitated; or fixed and cryopreserved; or capacitated and cryopreserved; or liquefied, fixed and capacitated; or liquefied, fixed and cryopreserved; or liquefied, capacitated and cryopreserved; or liquefied, fixed, capacitated and cryopreserved.

In some embodiments, the method, further comprising performing a computer-aided sperm analysis (CASA) on the spermatozoa.

In some embodiments, the method further comprises purifying the spermatozoa having (i) the at least one metal concentration, or (ii) the dynamic or kinetic parameter, or both (i) and (ii), that fall within the predetermined range to obtain functional spermatozoa.

In some embodiments, the method further comprises fertilizing the purified functional spermatozoa with an egg, optionally via in vivo fertilization (IVF) or intracellular sperm injection (ICSI).

In one aspect, the present disclosure provides a kit for performing the methods as disclosed here.

In some embodiments, the kit comprises a buffer and instructions for performing the methods as disclosed herein.

### BRIEF DESCRIPTION OF THE DRAWINGS

FIG. 1 shows a quantitative analysis of multiple elements in single human sperm in different samples. By using a method as disclosed herein, the difference of multiple elements in different samples can be detected. The lowest sensitivity range for detection of multiple elements can reach to a single cell level at a concentration of ten billionths of a gram (ag).

FIG. 2 shows the example of samples with a relatively high content of essential trace elements detected in single human spermatozoon, using a method as disclosed herein. These findings can be used as reference standards and suggestions for supplementary elements can be made to people with the deficiencies of these essential elements.

FIG. 3 provides an example of a sample with a relatively high content of toxic elements in single human spermatozoon. Clinical detoxification treatment can be suggested for those samples of subjects with relatively high levels of the toxic elements.

FIG. 4 shows examples of samples with a relatively high content of elements such as Nickel (Ni) in single human spermatozoon.

FIG. 5 provides the unique characteristics of ICP-MS signal spikes for iron (Fe) and copper (Cu) of the specific elements in single human sperm. sc-ICP-MS signal dynamics of the iron (Fe) and copper (Cu) elements is detected in the same batch of single human sperm cells in the same samples A, B, and C. Signal dynamic include whole-peak dwell time, peak time of a spike signal, pre- or post-peak dwell time, dynamic raising tau constant and area before the peak, and dynamic tailing tau constant and area after the peak. These parameters are the characteristics of specific elemental signals of a given cell type with unique elemental profiles.

FIG. 6 provides the signal characteristics of essential macro elements (calcium and magnesium) in different types of cells.

FIG. 7 provides the signal characteristics of essential trace elements in single human sperm, including zinc (Zn), iron (Fe), copper (Cu), manganese (Mn), chromium (Cr), cobalt (Co), selenium (Se), etc.

FIG. 8 provides the signal characteristics of essential trace elements in mouse epididymal epithelial DC2 cells, including zinc (Zn), iron (Fe), copper (Cu), manganese (Mn), chromium (Cr), cobalt (Co), selenium (Se), etc.

FIG. 9 provides the signal characteristics of essential trace elements in human fetal kidney 293T cells, including zinc (Zn), iron (Fe), copper (Cu), manganese (Mn), chromium (Cr), selenium (Se), etc.

FIG. 10 provides the signal characteristics of essential trace elements in human cervical cancer Hela cells, including zinc (Zn), iron (Fe), manganese (Mn), chromium (Cr), etc.

FIG. 11 provides the signal characteristics of essential trace elements in human gastric cancer SNU-1 cells, including zinc (Zn), iron (Fe), copper (Cu), manganese (Mn), chromium (Cr), etc.

FIGS. 12A-12E provide signal characteristics of toxic elements in different cell types, including human sperm from semen (FIG. 12A); cultured mouse epididymal epithelial DC2 cells (FIG. 12B); cultured human fetal kidney 293T cells (FIG. 12C); cultured human cervical cancer Hela cells (FIG. 12D); and cultured human gastric cancer SNU-1 cells (FIG. 12E). Further research is under investigation on the biotoxicological significance and pathological significance of the signal values of these toxic elements and their kinetic characteristics as well as the prospects for clinical diagnosis.

FIGS. 13A-13D provide mean mass of elements in single normal human spermatozoon. FIG. 13A shows mean mass of elements detected in single human spermatozoon from the whole population in samples with a sperm density lower or higher than 1×10⁶ per ml of normal (marked as Original) or viable DGC-capacitated samples (marked as Viable DGC) with a density of 1×10⁶ per ml or lower. FIG. 13B shows mean mass of elements detected in single human spermatozoon from the whole population in normal samples (marked as Original (low)) or oligoasthenozoospermia sperm samples (marked as Oligoastheno), both of which having a density of 1×10⁶ per ml or lower. FIG. 13C shows fold change of the elemental mean mass of samples of high sperm density over low sperm density. FIG. 13D shows fold change of the elemental mean mass of capacitated-DGC stimulated samples over samples without capacitation of either normal or oligoasthenozoospermia sperm cells. Data were represented as mean±SEM (n=3-30 samples), three-way ANOVA, *P<0.05, **** P<0.0001. ND: not determined due to out of detection range.

FIG. 14A provides correlation of mean mass of K versus Na in human spermatozoa with high or low density and with normal or oligoasthenozoospermia (Oligoastheno.) samples. Arrows indicate inversely correlation of K and Na. FIGS. 14B-14C provide comparison of mean mass of elements detected in human sperm using sc-ICP-MS and conventional digestion ICP-MS measurement. FIG. 14B shows mean mass of elements detected in single human spermatozoon of each population of normal or viable DGC samples with a density of 1×10⁶ per ml or lower or control normal spermatozoa or oligoasthenospermia samples. FIG. 14C shows mean mass of elements detected in batch of digested human sperm samples as in FIG. 14B. Data are represented as mean±SEM (n=3-30 samples), three-way ANOVA, **** P<0.0001. ND: not determined due to out of detection range.

FIG. 15 provides comparison of the mean mass of elements in single human spermatozoon (having a density lower than 1×10⁶ per ml) and other somatic culturing cells, including mouse WT epididymal epithelial DC2 cells, human embryonic fetal kidney 293T cells, human cervical cancer HeLa cells, human gastric cancer SNU-1 cells. Data were represented as mean±SEM (n=3-30 samples), two-way ANOVA, *P<0.05, ****P<0.0001. ND: not determined due to out of detection range.

FIGS. 16A-16C provide correlation of mean mass of elements versus Ca in single human spermatozoon of normal and oligoasthenozoospermia subjects. Mean mass of essential macro-elements (Na, K and Mg, FIGS. 16A), essential trace-elements (Co, Cr, Cu, Fe, Mn, Se and Zn, FIGS. 16B), and other or toxic elements (As, Al, Ag, Cd, Hg, Ni and Pb, FIG. 16C) as per sperm versus Ca mean mass in single human sperm of samples prepared from original semen (marked as original) or the normal viable DGC capacitated sperm in a Human tubal fluid (HTF) solution. Data points were obtained from the mean of at least three cells per each samples at a cell density of lower than 1×10⁶ per ml of normal or oligoasthenozoospermia spermatozoa diluted from original semen or after the DGC-capacitation treatment. Arrows indicate inversely correlation potential between elements and Ca. Lines indicate the data obtained from the same samples. ND: not determined due to out of detection range.

FIGS. 17A-17C provide correlation of mean mass of elements versus Ca in single human spermatozoon at a high or low cell density. Mean mass of essential macro-elements (Na, K and Mg, FIGS. 17A), essential trace-elements (Co, Cr, Cu, Fe, Mn, Se and Zn, FIGS. 17B), and other or toxic elements (As, Al, Ag, Cd, Hg, Ni and Pb, FIG. 17C) as per sperm versus Ca mean mass in single human sperm of samples prepared from original semen (marked as original) or the normal viable DGC capacitated sperm in a HTF solution. Data points were obtained from the mean of at least three cells per each samples at a cell density of higher or lower than 1×10⁶ per ml of normal or oligoasthenozoospermia spermatozoa diluted from original semen or after the DGC-capacitation treatment. Lines indicate the data obtained from the same samples.

FIGS. 18A-18C provide correlation of mean mass of elements versus Ca in single mouse epididymal epithelial DC2 cells. The mean mass correlation of essential macro-elements (Na, K and Mg, FIG. 18A), essential trace-elements (Co, Cr, Cu, Fe, Mn, Se and Zn, FIG. 18B), and other or toxic elements (As, Al, Ag, Hg, Ni and Pb, FIG. 18C) versus Ca as per single ICP-MS signal spike of aspirated single cells. Data points were obtained from the mean of at least three cells per sample. Lines indicate the data obtained from the same samples. ND: not determined due to out of detection range.

FIGS. 19A-19C provide correlation of mean mass of elements versus Ca in human embryonic fetal kidney HEK293T cells. Mean mass of essential macro-elements (Na, K and Mg, FIG. 19A), essential trace-elements (Co, Cr, Cu, Fe, Mn, Se and Zn, FIG. 19B), and other or toxic elements (As, Al, Ag, Hg, Ni and Pb, FIG. 19C) per cell versus Ca in aspirated single cells. Data points were obtained from the mean of at least three cells per sample. Lines indicate the data obtained from the same samples.

FIGS. 20A-20C provide correlation of mean mass of elements versus Ca in human cervical cancer HELA cells. Mean mass of essential macro-elements (Na, K and Mg, FIG. 20A), essential trace-elements (Co, Cr, Cu, Fe, Mn, Se and Zn, FIG. 20B), and other or toxic elements (As, Al, Ag, Hg, Ni and Pb, FIG. 20C) per cell versus Ca in aspirated single cells. Data points were obtained from the mean of at least three cells per sample. Lines indicate the data obtained from the same samples.

FIGS. 21A-21C provide correlation of mean mass of elements versus Ca in human gastric cancer SNU-1 cells. Mean mass of essential macro-elements (Na, K and Mg, FIG. 21A), essential trace-elements (Co, Cr, Cu, Fe, Mn, Se and Zn, FIG. 21B), and other or toxic elements (As, Al, Ag, Hg, Ni and Pb, FIG. 21C) per cell versus Ca in aspirated single cells. Data points were obtained from the mean of at least three cells per sample. Lines indicate the data obtained from the same samples.

FIG. 22 provides frequency distribution characteristics of mean mass of various elements detected in single human spermatozoon. Normal or oligosasthenozoospermia samples prepared directly from normal original semen (marked as Normal/Original or Abnormal/Original) versus those viable spermatozoa treated with a standard procedure of density gradient centrifugation (marked as Normal/Capacitated) in a HTF solution for sperm to undergo capacitation during centrifugation (marked as Abnormal/Capacitated). ND: not determined due to out of detection range. Data were detected at cell density of human sperm of less than 1×10⁶ per ml in single human spermatozoon, and represented as mean±SEM (n=3-30 individual samples), two-way ANOVA, *P<0.05, **P<0.01, ***P<0.001, ****P<0.0001.

FIGS. 23A-23B provide association of elementomic mean mass frequency patterns with oligoasthenozoospermia risk. Fe element analysis was used as an example to show that the detailed frequency distribution analysis of mean mass of Fe detected in single human sperm cells can reveal additional elemental bioavailability information in the identification of sub-population of sperm cells (FIG. 23B), which was not revealed if only the mean mass of the element was analyzed as a whole population for the single human spermatozoon samples (FIG. 23A). Dot plots show tracings of the entire defined spermatozoa population of individual preparation of normal semen, with or without capacitation challenge. Arrows indicate the subpopulations with distinct lower or higher Fe-contented of cells detected from the same sample population. Normal sperm without capacitation (Normal/Original); capacitated normal sperm (Normal/Capacitated); oligoasthenozoospermia without capacitation (Abnormal/Original); capacitated oligoasthenozoospermia (Abnormal/Capacitated). Data in FIG. 23A are represented as mean±SEM (n 3-30 individual samples). Two-way ANOVA, ****P<0.0001.

FIGS. 24A-24B provide association of element-specific kinetic parameters of ICP-MS signals with oligoasthenozoospermia risk. FIG. 24A provides a summary of the sc-ICP-MS elemental signal kinetics of the specific elements of original or DGC-capacitation stimulated normal or oligoasthenozoospermia human spermatozoa, including pre- or post-peak dwell time and pre- or post-peak tau constant of the ICP-MS signal spikes for iron (Fe), copper (Cu) and zinc (Zn). FIG. 24B provides a summary of the sc-ICP-MS elemental signal kinetics (pre- or post-peak dwell time and pre- or post-peak tau constant) of the specific elements of human spermatozoa with normal motility parameters, including Cr, Fe, Zn, Cu, Mn and Se. Data are represented as mean±SEM (n>3 individual samples). Two-way ANOVA, *P<0.05, **P<0.01, ***P<0.001, ****P<0.0001.

FIG. 25 provides examples of the unique element ICP-MS spike kinetic characteristics of essential elements, including Zn, Fe, Cu, Cr and Mn, on the basis of the sc-ICP-MS spike signals determined in different cell types, including single human sperm cells, cultured mouse epididymal epithelial DC2 cells, cultured human fetal kidney HEK293T cells, cultured human cervical cancer HeLa cells, and cultured human gastric cancer SNU-1 cells. The parameters, expressed in ms, include dwell time of the signal spikes of specific element in particular cell type, dwell time consisting of the pre-peak time (T₀-to-Tₚₑₐₖ) and the post-peak time (Tₚₑₐₖ-to-T_{end}). Data are represented as mean±SEM (n>3 individual samples). Two-way ANOVA, *P<0.05, **P<0.01, ***P<0.001, ****P<0.0001.

FIGS. 26A-26B show the morphology of sperm after spray chamber and sperm smear. To clarify the effect of the loading system of single cell ICP-MS on sperm, the sperm morphology was compared of the sperm that passed the nebulizer (FIG. 26A) and the sperm smear (FIG. 26B). The figures show that there was no significant difference in sperm morphology.

FIGS. 27A-27I provide dynamic calcium (Ca) flux analysis during capacitation indicating calcium overload in live sterile spermatozoa of Pmca4 KO mice and WT mice. FIG. 27A shows Ca content (attogram per cell, 10⁻¹⁸) in single sperm cells at different time points of capacitation analyzed by sc-ICP-MS. FIGS. 27B-27F show frequency distribution of calcium mass in single sperm cells at the indicated time points (FIG. 27B, 0 hour; FIG. 27C, 0.5 hour; FIG. 27D, 1 hour; FIG. 27E, 1.5 hour; and FIG. 27F, 2 hour). FIGS. 27G-27H show skewness (FIG. 27G) and kurtosis (FIG. 27H) of the frequency distribution patterns. FIG. 27I shows the range of mean mass content (in attogram) of Ca in single live sperm cells at different time points under the capacitation conditions and analyzed by sc-ICP-MS in the same single sperm population of Pmca4 KO and WT mice. Data herein and in subsequent figures show the means (±SD) of three mice per group. *P<0.05, **P<0.01 and ****P<0.0001, 2-way ANOVA.

FIGS. 28A-28I show dynamic zinc (Zn) flux analysis during capacitation of live mouse sperm as revealed by sc-ICP-MS. FIG. 28A provides moles of Zn in single sperm cells of Pmca4 KO and WT mice at different time points in capacitation conditions. FIGS. 28B-28F show frequency distribution of Zn) content of single sperm cells at the indicated time points (FIG. 28B, 0 hour; FIG. 28C, 0.5 hour; FIG. 28D, 1 hour; FIG. 28E, 1.5 hour; and FIG. 28F, 2 hour). FIGS. 28G-28H show skewness (FIG. 28G) and kurtosis (FIG. 28H) of the frequency distribution patterns. FIG. 28I shows the range of mean mass content (in attogram) of zinc in single live sperm cells at different time points under the capacitation conditions and analyzed by sc-ICP-MS in the same single sperm population of Pmca4 KO and WT mice. *P<0.05 of 2-way ANOVA.

FIGS. 29A-29I show dynamic iron (Fe) flux analysis during capacitation as revealed by sc-ICP-MS. FIG. 29A provides moles of Mn in single sperm cells of Pmca4 KO and WT mice at different time points of capacitation. FIGS. 29B-29F show frequency distribution of Fe content of single sperm cells at the indicated time points (FIG. 29B, 0 hour; FIG. 29C, 0.5 hour; FIG. 29D, 1 hour; FIG. 29E, 1.5 hour; and FIG. 29F, 2 hour). FIGS. 29G-29H show skewness (FIG. 29G) and kurtosis (FIG. 29H) of the frequency distribution patterns. FIG. 29I shows the range of mean mass content (in attogram) of Fe in single live sperm cells at different time points under the capacitation conditions and analyzed by sc-ICP-MS in the same single sperm population of Pmca4 KO and WT mice. *P<0.05 and **P<0.01, 2-way ANOVA.

FIG. 30A-30I show dynamic copper (Cu) flux analysis during capacitation as revealed by sc-ICP-MS. FIG. 30A provides moles of Mn in live single sperm cells of Pmca4 KO and WT mice at different time points of capacitation. FIGS. 30B-30F show frequency distribution of Cu content of single sperm cells at the indicated time points (FIG. 30B, 0 hour; FIG. 30C, 0.5 hour; FIG. 30D, 1 hour; FIG. 30E, 1.5 hour; and FIG. 30F, 2 hour). FIGS. 30G-30H show skewness (FIG. 30G) and kurtosis (FIG. 30H) of the frequency distribution patterns. FIG. 30I shows the range of mean mass content (in attogram) of Cu in single live sperm cells at different time points under the capacitation conditions and analyzed by sc-ICP-MS in the same single sperm population of Pmca4 KO and WT mice. ***P<0.001, 2-way ANOVA.

FIGS. 31A-31I show dynamic manganese (Mn) flux analysis during capacitation as revealed by sc-ICP-MS. FIG. 31A provides moles of Mn in live single sperm cells of Pmca4 KO and WT mice at different time points of capacitation. FIGS. 31B-31F show frequency distribution of manganese (Mn) content of single sperm cells at the indicated time points (FIG. 31B, 0 hour; FIG. 31C, 0.5 hour; FIG. 31D, 1 hour; FIG. 31E, 1.5 hour; and FIG. 31F, 2 hour). FIGS. 31G-31H show skewness (FIG. 31G) and kurtosis (FIG. 31H) of the frequency distribution patterns. FIG. 31I shows the range of mean mass content (in attogram) of Mn in single live sperm cells at different time points under the capacitation conditions and analyzed by sc-ICP-MS in the same single sperm population of Pmca4 KO and WT mice. *P<0.05 and **P<0.01, 2-way ANOVA.

FIGS. 32A-32I show dynamic selenium (Se) flux analysis during capacitation as revealed by sc-ICP-MS. FIG. 32A provides moles of Mn in live single sperm cells of Pmca4 KO and WT mice at different time points of capacitation. FIGS. 32B-32F show frequency distribution of selenium (Se) content of single sperm cells at the indicated time points (FIG. 32B, 0 hour; FIG. 32C, 0.5 hour; FIG. 32D, 1 hour; FIG. 32E, 1.5 hour; and FIG. 32F, 2 hour). FIGS. 32G-32H show skewness (FIG. 32G) and kurtosis (FIG. 32H) of the frequency distribution patterns. FIG. 32I shows the range of mean mass content (in attogram) of Se in single live sperm cells at different time points under the capacitation conditions and analyzed by sc-ICP-MS in the same single sperm population of Pmca4 KO and WT mice. *P<0.05, 2-way ANOVA.

FIGS. 33A-33F show the ratio of the content of each element against the content of nickel (Ni) ions in the same single sperm population of Pmca4 KO and WT mice. Content ratios are plotted of the moles of calcium (Ca, FIG. 33A); iron (Fe, FIG. 33B); copper (Cu, FIG. 33C); zinc (Zn, FIG. 33D); manganese (Mn, FIG. 33E); and selenium (Se, FIG. 33F) against the molar contents of Ni ions in single cells of different samples of KO and WT sperm during capacitation process. *P<0.05 of 2-way ANOVA.

FIGS. 34A-34I show effect of cell density on the ICP-MS signal profile of single human sperm cells. FIGS. 34A-34G show different cell densities from 3×10² per ml to 2×10⁷ per ml of ICP-MS signal profile of single human sperm cells during the time-resolving scan time (the example showing the signals of Fe element). The ICP-MS intensity corresponding to ICP-MS ion counts of spike signals for the determined element. Inserts show the distribution of ICP-MS spike of Fe single events digitized with a sampling dwell time of 50-μs at various cell densities of human sperm. FIG. 34H is a graph plot showing ICP-MS spike events per second independent of sperm density at density lower than 2×10⁶ per ml (arrow). FIG. 34I show spike events were also independent of experimental time after mortalization of the samples.

### DETAILED DESCRIPTION

It is to be appreciated that certain aspects, modes, embodiments, variations and features of the present technology are described below in various levels of detail in order to provide a substantial understanding of the present technology.

As it would be understood, the section or subsection headings as used herein is for organizational purposes only and are not to be construed as limiting and/or separating the subject matter described.

### Definitions

The definitions of certain terms as used in this specification are provided below. Unless defined otherwise, all technical and scientific terms used herein generally have the same meaning as commonly understood by one of ordinary skill in the art to which this present technology belongs.

As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

All numerical designations, e.g., pH, temperature, time, concentration, and molecular weight, including ranges, are approximations which are varied (+) or (−) by increments of 1.0 or 0.1, as appropriate or alternatively by a variation of +/−15%, or alternatively 10% or alternatively 5% or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, comparative terms as used herein, such as high, low, increase, decrease, reduce, or any grammatical variation thereof, can refer to certain variation from the reference. In some embodiments, such variation can refer to about 10%, or about 20%, or about 30%, or about 40%, or about 50%, or about 60%, or about 70%, or about 80%, or about 90%, or about 1 fold, or about 2 folds, or about 3 folds, or about 4 folds, or about 5 folds, or about 6 folds, or about 7 folds, or about 8 folds, or about 9 folds, or about 10 folds, or about 20 folds, or about 30 folds, or about 40 folds, or about 50 folds, or about 60 folds, or about 70 folds, or about 80 folds, or about 90 folds, or about 100 folds or more higher than the reference. In some embodiments, such variation can refer to about 1%, or about 2%, or about 3%, or about 4%, or about 5%, or about 6%, or about 7%, or about 8%, or about 0%, or about 10%, or about 20%, or about 30%, or about 40%, or about 50%, or about 60%, or about 70%, or about 75%, or about 80%, or about 85%, or about 90%, or about 95%, or about 96%, or about 97%, or about 98%, or about 99% of the reference.

“Optional” or “optionally” means that the subsequently described circumstance may or may not occur, so that the description includes instances where the circumstance occurs and instances where it does not.

As used herein, “and/or” refers to and encompasses any and all possible combinations of one or more of the associated listed items, as well as the lack of combinations when interpreted in the alternative (“or”).

“Substantially” or “essentially” means nearly totally or completely, for instance, 95% or greater of some given quantity. In some embodiments, “substantially” or “essentially” means 95%, 96%, 97%, 98%, 99%, 99.5%, or 99.9%.

In some embodiments, the terms “first” “second” “third” “fourth” or similar in a component name are used to distinguish and identify more than one components sharing certain identity in their names. For example, “first population of cells” and “second population of cells” are used to distinguishing two populations.

As used herein, the term “spermatozoon” or “sperm” or any grammatical variation of each thereof refers to a male reproductive cell. In some embodiments, spermatozoa or spermatozoon refers to a single male reproductive cell. In some embodiments, spermatozoa refers to a plurality of male reproductive cells. Additionally or alternatively, the spermatozoa can be alive or dead. Additionally or alternatively, the spermatozoa can be motile or immotile. Additionally or alternatively, the spermatozoa can be capacitated or non-capacitated. Additionally or alternatively, the spermatozoa can be in the biological sample from a subject. In further embodiments, the spermatozoa can be liquefied. In other embodiments, the spermatozoa can be purified or enriched or both purified and enriched. Additionally or alternatively, the spermatozoa can be fixed. Additionally or alternatively, the spermatozoa can be cryopreserved.

Sperm are produced in the testis and undergo maturation in the epididymis. Following this, they undergo a series of functional activities in the female tract before they gain the ability to fertilize oocytes. Sperm capacitation is an essential functional activity for fertilization. Influx of calcium ions is required for the capacitation process to occur (see, for example, Navarrete et al., J Cell Physiol, 2015, 230, 1758-1769; and Ickowicz et al., Asian J Androl, 2012, 14, 816-821; Wassarman P M, Cell, 1999, 96:175-183).

Disturbance of calcium homeostasis in sperm by genetic deletion of the calcium efflux pump Plasma Membrane Ca²⁺-ATPase isoform 4 (Pmca4) in mice leads to defective sperm motility and male infertility, with accompanying increased resting level of intracellular calcium (see, for example, Okunade et al., J Biol Chem, 2004, 279, 33742-33750; and Schuh et al., J Biol Chem, 2004, 279, 28220-28226).

“Metal element” “element” and “metal” are used interchangeably, referring to all elements included in Groups 1 to 12 of the periodic table except for hydrogen, and all elements included in Groups 13 to 16 of the periodic table except for all the metalloid elements, C, N, P, O, S, and Se. In other words, the metal element becomes a cation when the metal element forms an inorganic compound with a halogen. In some embodiments, metal elements include metal elements in their elemental form as well as metal elements in an oxidized or reduced state, for example, when a metal element is combined with other elements in the form of compounds comprising metal elements. For example, metal elements can be in the form of hydrates, salts, oxides, as well as various polymorphs thereof, and the like.

The terms “sperm capacitation,” “capacitation,” or any grammatical variation thereof, as used herein, refer to the processing of sperm that allows the sperm to acquire the ability to fertilize an egg, such as to undergo acrosomal exocytosis and binding to and penetrating through the zona pellucida of an unfertilized egg (see, for example, Wassarman P M, Cell, 1999, 96:175-183). Completion of capacitation is manifested by the ability of sperm to bind to the zona pellucida and to undergo ligand-induced acrosomal reaction. In some embodiments, the capacitation refers to an in vivo processing. For example, sperm capacitation occurs naturally throughout the female reproductive tract. In some embodiments, the capacitation refers to an ex vivo or in vitro processing. For example, assisted reproductive techniques perform in vitro capacitation techniques to improve the chances of successful fertilization. There are different techniques to perform the capacitation step: simple washing, migration (swim-up), density gradients, and filter.

In some embodiments, the method to perform in vitro capacitation comprises, or consists essentially of, or yet further consists of one or more of the following:

(1) Simple wash: referring to a washing eliminating seminal plasma from a sample comprising spermatozoa. In some embodiments, the sample is centrifuged and then, the supernatant is eliminated.

(2) Migration (swim-up): referring to selecting sperm cells based on their ability to move upwards (“to swim”) from the bottom to the top of the tube. An non-limiting example is provided herein: firstly, centrifugation takes place and seminal plasma is eliminated; then, 0.5-1 ml of culture medium or another buffer is added at the top and after the incubation period at 37° C., the best motile spermatozoa would have ascend from the bottom to the top of the tube (healthy spermatozoa go to the culture medium); and in order to obtain the fraction rich in spermatozoa, the top layer is collected.

(3) Density gradients: referring to a density gradient centrifugation of a sample comprising sperm cells. An non-limiting example is provided herein: a tube is filled with layers of liquids of different densities and semen is placed on the top layer; then, the tube goes through a centrifugation to filter cell debris and non-motile cells; after the centrifugation, healthy sperm are on the very bottom layer of the liquid in the tube, while debris and non-motile spermatozoa are in upper layers; and at the end, all the cells would arrive to the bottom, but those with more motility would arrive sooner. This procedure is often called just the “Percoll method”, since Percoll was frequently used as the density medium, but other density mediums can also be used.

(4) Filtration: referring to filtrating a sample comprising sperm cells. A non-limiting example comprises, or consists essentially of, or yet further consists of a filter that does not allow every sperm to pass; and only spermatozoa with better motility would pass through the filter.

(5) Others: such as in vitro fertilization (IVF), physiological intracytoplasmic sperm injection (PICSI), sperm penetration assay (SPA), magnetic-activated cell sorting (MACS) or microfluidic chips, each optionally obtaining motile sperm.

Methods to determine sperm capacitation are known in the art, for example, the most common sperm-zona pellucida binding tests currently utilized are the hemizona assay (or HZA) and a competitive intact-zona binding assay. A hemizona assay measures the ability of sperm to undergo capacitation and bind to an oocyte. Sperm is incubated with dead oocytes which are surrounded by the zona pellucida, and a cellular coating of oocytes. Capacitated sperm bind to the zona and the number of sperm binding is counted microscopically. This number correlates with the number of normal capacitated sperm in a sample and with fertility of a sperm sample. For example, see Cross et al., Gamete Res. 1986; 15:213-26 and Wassarman P M, Cell, 1999, 96:175-183).

Human tubal fluid (HTF) buffer refers to a synthetic solution mimicking the composition of the fluid found in human fallopian tubes. It is suitable for procedures, such as the retrieval, handling and transfer of human gametes and embryos. In some embodiments, a HTF buffer uses a sodium bicarbonate buffering system, which is appropriate for those procedures requiring the use of a carbo dioxide atmosphere during incubation. In some embodiments, a HTF buffer as used herein also refers to a modified HTF buffer using a different buffer system, such as a combined sodium bicarbonate/HEPES buffer. In some embodiments, the buffer system provides maintenance of physiological pH (for example 7.2 to 7.4).

As used herein, the term liquefaction or any grammatical variation thereof, such as liquefied, refers to a process when the gel formed by proteins from the seminal vesicles is broken up and the semen becomes more liquid. In some embodiments, placing a semen sample at room temperature or at 37° C. for about 15 minutes to about 60 minutes (such as about 30 minutes to about 60 minutes) liquefies the sample. In further embodiments, during liquefaction, continuous gentle mixing or rotation of the sample container on a two-dimensional shaker can help to produce a homogeneous sample. Additionally or alternatively, mechanical mixing (such as repeated pipetting or gentle passage through a blunt gauge 18 or gauge 19 needle attached to a syringe) or enzymatic digestion (such as digestion by bromelain or other proteolytic enzyme) or both can be used for liquefaction.

“Fixing” refers to a process that maintains the structure of cells and/or sub-cellular components such as cell organelles (e.g., nucleus). Fixing modifies the chemical or biological structure cellular components by, e.g., cross-linking them. Fixing may cause whole cells and cellular organelles to resist lysis. “Fixative” refers to an agent such as a chemical or biological reagent that fixes cells. A fixative may disable cellular proteolytic enzymes and nucleases. Examples of fixatives include aldehydes (e.g., formaldehyde, or paraformaldehyde (PFA)), alcohols, and oxidizing agents. Examples of suitable fixatives are presented in US Patent Application Publication 2010/0184069, filed Jan. 19, 2010, and in US Patent Application Publication No. 2010/209930, filed Feb. 11, 2010.

As used herein, the term “cryopreservation” refers to stably maintaining cells for a long period of time via freezing. In some embodiments, cell cryopreservation is performed to freeze and preserve cells before losing their intrinsic characteristics, or to use them when needed, or both. Cell cryopreservation may be performed by contacting cells to be cryopreserved with a cryprotectant, and optionally a cell culture medium to prevent cell damage due to a cryoprotectant, thereby improving safety and stability in cryopreservation of cells. The term “cryoprotectant” as used herein refers to a substance used when the cells are stored below 4° C., or at an ultra-low temperature of −80° C. to −200° C. In particular, the substance can minimize the formation of ice crystals and cell damage due to imbalance of ion and osmotic pressure inevitably accompanied by freezing and thawing processes.

The terms “isolated”, “isolating” or “isolation” and “purified”, “purifying” or “purification” as used herein with reference to molecules does not refer to absolute purity. Rather, “purified”, “purifying” or “purification” refers to a substance in a composition that contains fewer substance species in the same class (e.g., cell species) other than the substance of interest in comparison to the sample from which it originated.

As used herein, CASA is an acronym for computer-aided sperm analysis or computer-assisted sperm analysis, referring to the process analyzing concentration, motility, cell morphology, or any combination thereof of a semen sample with the help of a computer. Several manufacturers produce CASA systems, such as MICROPTIC S. L. and Hamilton Thorne. Additionally, uses of CASA to measure sperm motility and concentration are described in Sections 3.5.2 and 3.5.3 of WHO, 2010, respectively.

As used herein, the term “IVF” or “in-vitro fertilization” refers to fertilization of an oocytes with a sperm outside of an organism. IVF may also refer to a technique, whereby oocytes and spermatozoa are mixed in the laboratory to achieve fertilization.

As used herein, the term “intracytoplasmic sperm injection” (ICSI) refers to a process in which a sperm cell is injected directly into an ovum, such as a human ovum, so as to promote fertilization of the ovum and zygote formation. The sperm cell may be injected into the ovum, for instance, by piercing the oolemma with a microinjector so as to deliver the sperm cell directly to the cytoplasm of the ovum. ICSI procedures useful in conjunction with the compositions and methods described herein are known in the art and are described, for instance, in WO 2013/158658, WO 2008/051620, and WO 2000/009674, among others.

Inductively coupled plasma mass spectrometry (ICP-MS) refers to a type of mass spectrometry (MS) that uses an inductively coupled plasma (ICP) to ionize the sample. It is known and used for its ability to detect metals and several non-metals in liquid samples at very low concentrations. Once a sample (such as a single cell as disclosed herein) enters the ICP, the sample is vaporized, atomized and ionized, forming a cloud of elemental ions. The generated ions are directed from the ICP toward the mass analyzer through a pressure-reduction interface that reconciles the pressure difference between the atmospheric-pressure ICP and the low pressure (e.g., 10-6 mbar) mass analyzer. In some embodiments, ion optics are used to efficiently transmit ions to the mass analyzer. The mass analyzer uses electric or magnetic field or both fields to separate ions according to their mass-to-charge ratio (m/Q) before they strike a detector. The generated data show the number of ions recorded at each m/Q. The m/Q can be used to determine the elemental identity of an ion, and the number of ions to determine element concentration. The cloud of elemental ions created from a sample in the ICP source generates a very fast transient signal (which is referred to herein as a “signal spike” or a “spike”), with a total duration (which is referred to herein as “dwell time”). Scanning analyzers generally target one or two elements, whereas time-of-flight (TOF) mass analyzers are able to record the entire mass spectrum (all m/Q values) for each sample. For any recorded isotope (m/Q value), the total ion signal observed during the duration of the transient particle signal is proportional to the mass of that element in the sample. The frequency of particle events (transient signal spikes) detected by the ICP-MS is proportional to the particle number concentration in the introduced liquid sample. Continuous signal regions that do not contain spikes (single particle detection events) represents the concentration of the sample fraction present in dissolved form. The terms “peak” and “spectral peak” refer to a peak in the output from a mass spectrum, such as a peak of the spike.

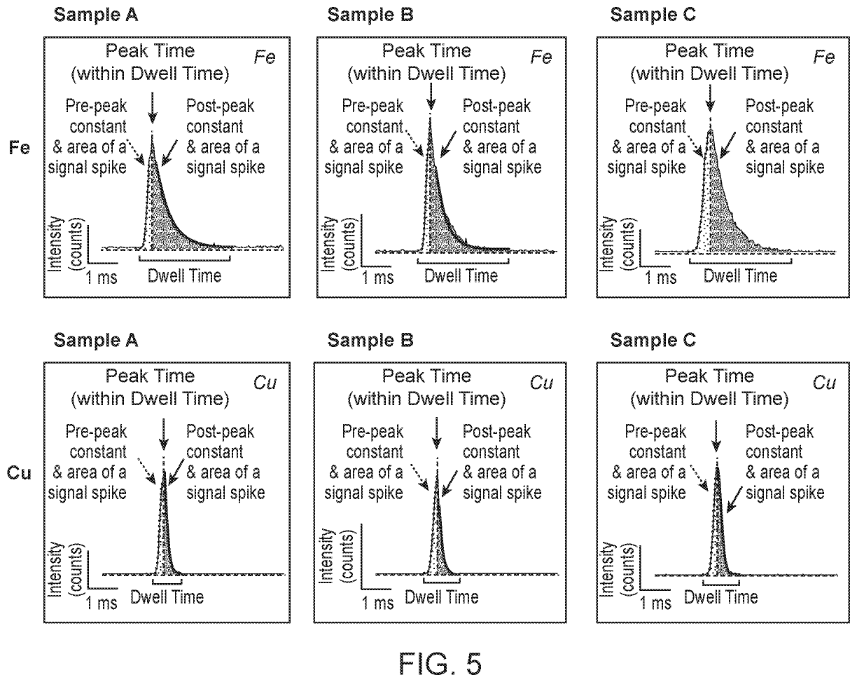

In some embodiments, a dynamic parameter refers to a parameter whose change over time of interest, rather than a parameter whose absolute vale at a specific point in time is of interest, such as raising tau constant before the peak (i.e., the amount of time elapsed from the beginning of the spike to the peak in a manner following an alpha exponential functional algorithm, which is also called standard exponential; the fitting of functional algorithm should not only limited to this function), dynamic area before the peak (i.e., the area under the spike before the peak), tailing tau constant after the peak (i.e., the amount of time elapsed from the peak to the end of the spike in a manner following an alpha exponential functional algorithm, which is called standard exponential; but the fitting of functional algorithm should not only limited to this function), dynamic area after the peak (i.e., the area under the spike after the peak), and area of the peak or the spike (i.e., the area under the spike). See, the illustration in FIG. 5.

In some embodiments, the alpha exponential function comprises the algorithm:

$$f ( t ) = {\sum }_{i = 1}^{n} {A}_{i} {e}^{- t / τ i} + C .$$

In some embodiments, the dynamic parameters can be calculated using functional algorithms. In some embodiments, the functional algorithms are selected from Exponential alpha; Exponential cumulative probability, Exponential log probability, Exponential power, Exponential probability, Exponential product, Exponential sloping baseline, Exponential standard, Exponential weighted, Exponential weighted/constrained, Guassian, Binomial, Polynomial, Boltzman charge-voltage, Boltzman shifted, Boltzman standard, Boltzman Z-delta (ascending), Boltzman Z-delta (descending).

In some embodiments, a kinetic parameter refers to a parameter describing kinetics of a spike, such as dwell time (i.e., the amount of time elapsed during the spike), pre-peak dwell time (i.e., the amount of time elapsed during the spike and before the peak), post-peak dwell time (i.e., the amount of time elapsed during the spike and after the peak), and peak time (i.e., the amount of time elapsed for the spike to reach the peak in proportion of the spike dwell time). See, the illustration in FIG. 5. In some embodiments, the term “kinetic parameter” is used interchangeably with “dynamic parameter.”

As used herein, a first level “comparable to” a second level refers to the first level is substantially similar compared to the second level. For example, the first level is about 50% to about 2 folds (or any percentage or fold or range there between) of the second level. In some embodiments, the first level is about 80% to about 120% (or any percentage or range there between, such as about 90%, about 91%, about 92%, about 93%, about 94%, about 95%, about 96%, about 97%, about 98%, about 99%, about 100%, about 101%, about 102%, about 103%, about 104%, about 105%, about 106%, about 107%, about 108%, about 109%, or about 110%) of the second level. In some embodiments, a first level corresponding to a second level refers to the first level comparable to the second level. Additionally or alternatively, a first range corresponding to a second range refers to the upper or lower limit or both the upper and the lower limits of the first range comparable to that of the second range.

As used herein, a predetermined level, a predetermined range, a reference level, a reference range, and a reference refer to a range of the level in a control sample. In some instances, the control sample is obtained from a healthy subject, e.g., a subject who does not have one or more of a disease or condition. In further instances, the control sample is a reference sample specific to the laboratory facility and the predetermined level is established for a particular assay of interest by the laboratory facility that carries out the particular assay of interest. In some cases, the predetermined level is measured utilizing a sample as disclosed herein. A skilled artisan would appreciate that the level is influenced by the assay, by the subject's age, and by the health of the subject.

As used herein, a biological sample, or a sample, is obtained from a subject. Exemplary samples include, but are not limited to, cell sample, cellular-derived particles (for example, sperm-derived exosomes, non-sperm derived exosomes, epididymosomes, membrane-enveloped particles, lipid-enveloped biological particles, cytoplasmic droplets, and/or cellular organelles such as mitochondria, lysosomes, endosomes, endoplasmic reticulum, smooth endoplasmic reticulum, rough endoplasmic reticulum, cilia, primary cilia, ribosome, Golgi apparatus, nucleus, chromatin, chromosome, mesosome, peroxisome, microtubules, actin filaments, and/or intermediate filaments) tissue sample, tumor biopsy, liquid samples such as blood and other liquid samples of biological origin, including, but not limited to, ocular fluids (aqueous and vitreous humor), peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, female ejaculate, sweat, tears, cyst fluid, pleural and peritoneal fluid, pericardial fluid, ascites, lymph, chyme, chyle, bile, interstitial fluid, menses, pus, sebum, vomit, vaginal secretions/flushing, synovial fluid, mucosal secretion, stool water, pancreatic juice, lavage fluids from sinus cavities, bronchopulmonary aspirates, blastocyl cavity fluid, or umbilical cord blood. In some instances, the sample is a semen sample. In some instances, the sample is a cellular-derived particles (for example, sperm-derived exosomes, non-sperm derived exosomes, epididymosomes, membrane-enveloped particles, lipid-enveloped biological particles, cytoplasmic droplets, and/or cellular organelles such as mitochondria, lysosomes, endosomes, endoplasmic reticulum, smooth endoplasmic reticulum, rough endoplasmic reticulum, cilia, primary cilia, ribosome, Golgi apparatus, nucleus, chromatin, chromosome, mesosome, peroxisome, microtubules, actin filaments, and/or intermediate filaments).

As used herein, the term “animal” refers to living multi-cellular vertebrate organisms, a category that includes, for example, mammals and birds. The term “mammal” includes both human and non-human mammals.

The term “subject,” “host,” “individual,” and “patient” are as used interchangeably herein to refer to animals, typically mammalian animals. Any suitable mammal can be treated by a method described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments, a mammal is a human. A mammal can be any age or at any stage of development (e.g., an adult, teen, child, infant, or a mammal in utero). A mammal can be male or female. In some embodiments, a subject is a human. In some embodiments, a subject has or is diagnosed of having or is suspected of having a disease.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. In one aspect, treatment excludes prophylaxis.

In some embodiments, the term “disease” or “disorder” as used herein refers to infertility, a status of being diagnosed with infertility, a status of being suspect of having infertility, or a status of at high risk of having infertility. In some embodiments, the disease is of a male subject.

As used herein, the term “infertility” refers to the inability or diminished ability to conceive or produce offspring. Infertility can be present in either male or female. As used herein, male infertility refers to a male's inability to cause pregnancy in a fertile female. Male infertility is commonly due to deficiencies in the semen, and semen quality may be used as a surrogate measure of male fecundity. In some embodiments, the infertility comprises, or consists essentially of, or yet further consists of idiopathic infertility, such as asthenozoospermia, asthenoteratozoospermia, necrozoospermia, oligoteratozoospermia, oligozoospermia, oligoasthenozoospermia, oligoasthenoteratozoospermia, or teratozoospermia.

As used herein, the term “fertile” refers to a male subject having motile or viable or both motile and viable sperm and which therefore have a fairly high probability for initiating a pregnancy. In some embodiments, the term “infertile” refers to failure to establish a clinical pregnancy after twelve months of regular, unprotected sexual intercourse.

The term “unexplained” or “idiopathic” infertility is applied to either or both subjects of every couple whose failure to conceive within a period of at least one year, is clinically inexplicable and where fertility testing of both partners has revealed no identifiable cause. When a couple is found to be “normal” in a standard fertility evaluation (for example, female—ovulatory and normal post-coital tests, timed endometrial biopsy, hysterosalpingogram, laparoscopy; male—“normal” sperm analysis, with sperm concentration greater than 20 million/ml on at least two occasions, total sperm numbers of 40 million or more, sperm motility greater than 50%, and normal morphology in more than 30% of the spermatozoa), and the couple has had a history of involuntary infertility for at least 1 year, a human couple or either partner of the couple is diagnosed unsatisfactorily as “unexplainably” or “idiopathic” infertile. Such couples often undergo numerous invasive, protracted and expensive assisted reproductive technology attempts in their pursuit of pregnancy, such as IVF.

Asthenozoospermia refers to a condition characterized by reduced sperm motility compared to a healthy subject or the average level or the range of a population of healthy subjects. In some embodiments, a male subject having an asthenozoospermia shows a percentage of progressively motile (PR) spermatozoa below a predetermined level.

Asthenoteratozoospermia refers to a condition characterized by reduced sperm motility compared to a healthy subject or the average level or the range of a population of healthy subjects, and abnormal morphology of the sperm. In some embodiment, a male subject having an asthenoteratozoospermia shows percentages of both progressively motile (PR) and morphologically normal spermatozoa below predetermined levels.

Necrozoospermia refers to a condition where spermatozoa in semen are either immobile or dead or both. In some embodiments, a male subject having necrozoospermia shows lower percentage of live, or higher percentage of immotile, or both lower percentage of live and higher percentage of immotile, spermatozoa in the ejaculate, compared to a healthy subject or the average level or the range of a population of healthy subjects.

Oligoteratozoospermia refers to a combination of oligozoospermia and teratozoospermia. In some embodiments, a male subject having oligoteratozoospermia shows a total number (or a concentration) of spermatozoa, and a percentage of morphologically normal spermatozoa, below predetermined levels.

Teratozoospermia refers to a condition characterized by the presence of morphologically abnormal spermatozoa. In some embodiments, a male subject having teratozoospermia shows a percentage of morphologically normal spermatozoa below a predetermined level.

Oligozoospermia refers to a condition where lower numbers of spermatozoa are produced in an ejaculation compared to a healthy subject or the average level or the range of a population of healthy subjects. In some embodiments, a male subject having oligozoospermia shows a total number (or concentration) of spermatozoa below a predetermined level.

Oligoasthenozoospermia refers to a combination of oligozoospermia and asthenozoospermia. In some embodiments, a male subject having oligoasthenozoospermia shows a total number (or concentration) of spermatozoa, and a percentage of progressively motile (PR) spermatozoa, below predetermined levels.

Oligoasthenoteratozoospermia refers a combination of oligozoospermia, asthenozoospermia and teratozoospermia. In some embodiments, a male subject having oligoasthenoteratozoospermia shows a total number (or concentration) of spermatozoa, and percentages of both progressively motile (PR) and morphologically normal spermatozoa, below predetermined levels.

A simple system for grading motility is recommended by WHO, 2010 that distinguishes spermatozoa with progressive or non-progressive motility from those that are immotile. The motility of each spermatozoon is graded as follows: Progressive motility (PR): spermatozoa moving actively, either linearly or in a large circle, regardless of speed. Non-progressive motility (NP): all other patterns of motility with an absence of progression, e.g. swimming in small circles, the flagellar force hardly displacing the head, or when only a flagellar beat can be observed. Immotility (IM): no movement. In some embodiments, the predetermined level for PR is about 31% to about 34% or any percentage or range therebetween, such as about 31%, or about 32%, or about 33%, or about 34%. In some embodiments, the predetermined level for total motility (PR and NP) is about 38% to about 42% or any percentage or range therebetween, such as about 38%, or about 39%, or about 40%, or about 41%, or about 42%.

In some embodiment, the predetermined level for vitality (such as membrane-intact spermatozoa) is about 55% to about 63% or any percentage or range therebetween, such as about 55%, or about 56%, or about 57%, or about 58%, or about 59%, or about 60%, or about 61%, or about 62%, or about 63%.

In some embodiment, the predetermined level for sperm concentration is about 12×10⁶ to 16×10⁶ spermatozoa per ml or any concentration or range therebetween, such as about 12×10⁶ spermatozoa per ml, or about 13×10⁶ spermatozoa per ml, or about 14×10⁶ spermatozoa per ml, or about 15×10⁶ spermatozoa per ml, or about 16×10⁶ spermatozoa per ml.

In some embodiment, the predetermined level for total sperm number is about 33×10⁶ to 46×10⁶ spermatozoa per ejaculate or any concentration or range therebetween, such as about 33×10⁶ spermatozoa per ejaculate, or about 34×10⁶ spermatozoa per ejaculate, or about 35×10⁶ spermatozoa per ejaculate, or about 36×10⁶ spermatozoa per ejaculate, or about 37×10⁶ spermatozoa per ejaculate, or about 38×10⁶ spermatozoa per ejaculate, or about 39×10⁶ spermatozoa per ejaculate, or about 40×10⁶ spermatozoa per ejaculate, or about 41×10⁶ spermatozoa per ejaculate, or about 42×10⁶ spermatozoa per ejaculate, or about 43×10⁶ spermatozoa per ejaculate, or about 44×10⁶ spermatozoa per ejaculate, or about 45×10⁶ spermatozoa per ejaculate, or about 46×10⁶ spermatozoa per ejaculate.

In some embodiment, the predetermined level for morphologically normal spermatozoa in an ejaculate is about 3% to about 4% or any percentage or range therebetween, such as about 3%, or about 4%.

An infertility therapy refers to a treatment of infertility, such as a method, a composition, an active ingredient, or any combination thereof. A variety of infertility therapies include, but are not limited to, an assisted reproductive technology (ART), in vitro fertilization (IVF), ovarian hyperstimulation, controlled ovarian hyperstimulation, natural cycle in vitro fertilization, final maturation induction, transvaginal oocyte retrieval, egg and sperm preparation, co-inoculation, embryo culture, adjunctive medication, cycle-stimulation therapies, follicle-stimulating hormone (FSH) therapies, microdose gonadotropin-releasing hormone antagonist (GnRHa) flare therapies, antagonist (e.g., GnRHant) therapies, intracytoplasmic sperm injection (ICSI), augmented intracytoplasmonic sperm injection, mitochondrial augmented intracytoplasmic sperm injection and related female germline stem cell treatments (e.g., the AUGMENT™, OVAPRIME™ and OVATURE™ treatments offered by OvaScience, Inc. of Waltham, Mass.), adoption, and the like.

### MODES FOR CARRYING OUT THE DISCLOSURE

Elementomics in biomedical sciences: Metals play essential function in life. One third of proteins are believed to be metalloproteins and require metal cofactors, which usually are transition metals, such as copper (Cu), iron (Fe), cobalt (Co), chromium (Cr) or manganese (Mn) (Nolan, 2016, Science 352, 1055-1056; and Waldron et al., 2009, Nature 460, 823-830), as well as selenium (Se) element (Green, 2018, Cell 172, 389-390; and Kieliszek et al., 2021, Biol Trace Elem Res). Most of these transition metals or elements are redox active in biological systems, or bind to macromolecules that can be regulated by redox potential. Metal cofactors are also required for proper folding and other biological function of proteins, such as zinc (Zn) and calcium (Ca), which are among the most common cofactors (Bushmarina et al., 2006, Protein Sci 15, 659-671). Understanding of the entirety of metal and metalloid and elemental species within a cellular compartment or a cell or a tissue type, i.e. elemental profiling, is therefore important and is getting more and more attention in the field of life sciences (Miyashita et al., 2017, In Metallomics: Recent Analytical Techniques and Applications, Y Ogra, and T. Hirata, eds. (Tokyo: Springer Japan), pp. 107-124; Mounicou et al., 2009, Chem Soc Rev 38, 1119-1138; Nelson, 1999, Embo J 18, 4361-4371; Nolan; and Waldron et al., 2009, Nature 460, 823-830). The characteristics of elemental profile of a given biological system requires comprehensive analysis in a systemic approach by studying the element content, speciation and localization, as well as the entirety of associated biomolecular profile of how an element is sensed, stored, or used. For example, the bioavailability of a set of transition metals such as Cu, Fe and Zn, and the intracellular concentrations of their associated metallotheoneins are believed to be correlated with the expression of some protein levels and their distribution among the various cell compartments (Calvo et al., 2017, IUBMB Life 69, 236-245; Outten and O'Halloran, 2001, Science 292, 2488-2492; Ruttkay-Nedecky et al., 2013, Int J Mol Sci 14, 6044-6066; Sakulsak, 2012, International Journal of Morphology 30, 1007-1012; and Waldron et al., 2009, Nature 460, 823-830). Whereas albumin and transferrin are essential metallo-transporters in extracellular fluid or blood plasma and when necessary are internalized through endocytosis process and trafficked to the destinated cell compartments (Kawabata, 2019, Free Radic Biol Med 133, 46-54; and Waldron et al., 2009, Nature 460, 823-830). The distribution of these metals and their metalloproteins are therefore tightly controlled and might be functionally interconnected (Chang, 2015, Nat Chem Biol 11, 744-747; Duncan, 2009, Metal Ions in Life Sciences Vol. 5. Edited by Astrid Sigel, Helmut Sigel and Roland K. O. Sigel. 48, 7966-7967; Krezel and Maret, 2017, Int J Mol Sci 18; Mounicou et al.; Nelson; Sakulsak; and Tvrda et al., 2015, J Assist Reprod Genet, 2015, 32, 3-16). It is believed that each biological system is engineered to a specific set of the element-protein partnerships. With increasing evidence for bioinorganic function of metals in reproductive biology, the importance of metal ion homeostasis in sperm function and male fertility is well-documented. However, why each metal-protein partnership occurs in a given biology system, such as in spermatozoa, and the mechanisms for how this partnership is maintained under physiological conditions and how it is dysregulated in pathophysiological conditions are still largely unknown.

Recent advancement of single cell elementomics: Intact sperm function is the foundation of male reproductive health. Bio-metals play essential role in sperm function and male fertility and the homeostatic imbalance of trace elements is one of the key factors leading to male infertility (Ali et al., 2017, Biol Trace Elem Res 175, 244-253; Mirnamniha et al., 2019, Rev Environ Health 34, 339-348; Schmid et al., Human Reproduction, 2013, 28, 274-282; and Tvrda et al.). For example, dynamic calcium concentration in the state of sperm capacitation (Navarrete et al., J Cell Physiol, 2015, 230, 1758-1769). In addition, the effects of different elements such as Zn and Se can improve male fertility (Colagar et al., 2009, Nutr Res 29, 82-88; and Hawkes and Turek, 2001, J Androl 22, 764-772), while Cd can cause weakening of male fertility and the different concentration of nickel (Ni) has a dual effect on sperm motility on the sperm after capacitation have also been reported (de Angelis et al., 2017, Reprod Toxicol 73, 105-127; Kumar and Sharma, 2019, Rev Environ Health 34, 327-338; and Mirnamniha et al.). These inorganic elements exert their function in biological system with their unique physiochemical property and quantity. For the metals in macro amounts, it is usually those metals that are stable in ionic state, such as potassium (K), sodium (Na), Ca and magnesium (Mg) (Mirnamniha et al.). Those elements that play essential roles at moderate levels in reproduction and sperm biology are usually those transition metals, such as Fe, Cu and Zn, while some other essential metals can induce various physiological effects in a very trace amount, such as Co, Cr and Mn as well as rare earth elements (Marzec-Wroblewska et al., Arch Environ Contam Toxicol, 2015, 69, 191-201; Mirnamniha et al.; and Tvrda et al.). Some metalloids or heavy metals pollution introduce toxic effect on sperm function is known, like arsenic (As), cadmium (Cd), mercury (Hg) and lead (Pd) (Inhorn et al., 2008, Reprod Toxicol 25, 203-212; Wang et al., 2016, Sci Total Environ 571, 307-313; and Wang et al., 2017, Environ Pollut 224, 224-234).

In fact, ion content can also become the standard for evaluating sperm quality. Many studies have reported that cadmium and nickel cause male fertility to be weakened, selenium can improve fertility, and calcium is the key essential ion for sperm capacitation. See, for example, Ingold et al.; Kasperczyk et al.; Li et al., 2012a; Li et al., 2012b; Marzec-Wroblewska et al.; Schmid et al.; Tvrda et al.; and Zhao et al., 2017. Therefore, detecting the ion spectrum of single sperm provides a new angle and direction for the sperm quality evaluation method. On the other hand, there is still a lack of clinical ion spectrum analysis methods for single cells. Therefore, the establishment of a method to detect the ion spectrum of a single sperm and a single cell is of great significance for clinically improving sperm quality, cell function and providing reference values.

Interestingly, there is increasing evidence that sperm dysfunction is associated with elemental imbalance (see, for example, Schmid et al.; Marzec-Wroblewska et al.; and Kasperczyk et al., J Trace Elem Med Biol, 2015, 30, 153-159). Studies have reported that disordered trace metals such as cadmium, manganese and nickel are associated with male weakened fertility cases (see, for example, Li et al., 2012a; Li et al., 2012b, BMC Public Health, 2012, 12, 919; and Zhao et al., PLoS One, 2017, 12, e0186727), whereas selenium can improve fertility (see, for example, Ingold et al., Cell, 2018, 172, 409-422 e421). Moreover, imbalance between iron and copper homeostasis can cause increased oxidative stress and thus affect sperm cell viability and even sperm cell death (see, for example, Tvrda et al., JAssist Reprod Genet, 2015, 32, 3-16). Thus, the contents of elemental ions can be chosen as standard parameters for evaluating sperm quality. However, methods monitoring of dynamic elemental changes during sperm capacitation at the single cell level have not yet been reported.

Although it is ambitious, a comprehensive network on element-protein partnerships and their spatial-temporal distribution in compartments of sperm cells and reproductive tissues, as well as how this network is maintained and how the element-protein partnerships are interacted in a concerted manner, would enable a comprehensive understand the elemental biology in sperm and reproduction. However, there is no method reported for detecting metal ion concentrations, the so-called metallomics, in single sperm cells evaluating functional activity. Elemental availability, or elementomics in other term, particularly at the single cell level, is believed to be the first step to understand the specific set of the element-protein partnerships of sperm cells. In some embodiments, the aim of this disclosure was therefore to develop and validate a method for simultaneously determination the content of multiple elements and analysis of the elemental characteristics in human sperms, namely elementomic profiling.

A need for a method of elementomic analysis of sperm at single-cell level: The drive of developing the technology as disclosed herein is due to the main shortcomings of the existing element determination methods for biological materials, which require either large quantity of sample volume, or have a low detection sensitivity, few detection elements, long detection time, and a harsh sample preparation procedure which is tremendously different from the physiological conditions for inorganic elements functioning in biological system. For example, these analytical methods are usually applied to samples in solutions and comprise digesting organic samples into simpler inorganic form in solution using strong acids, which is tremendously different from the physiological conditions for inorganic elements functioning in biological system. Additionally, the main disadvantages of the existing element determination method “flame method” technology are low sensitivity, few detection elements, and long detection time. The methods available for analyzing elements in biological material include potentiometric, voltametric, atomic spectrometric, and X-ray fluorescence technologies (Brown and Milton, 2005, Trac-Trend Anal Chem 24, 266-274; and Zhao et al., 2014, Trends in plant science 19, 183-192). In clinical labs, atomic spectrometry is the common technique used for elemental analysis of human biological material, including flame atomic absorption spectrometry (FAAS), graphite furnace atomic absorption spectrometry (GFAS), inductively coupled plasma atomic emission spectrometry (ICP-AES) and inductively coupled plasma mass spectrometry (ICP-MS) (Bulska and Ruszczynska, 2017, Physical Sciences Reviews 2).

ICP-MS technique has been applied to evaluate the elemental composition of digested samples, including digested human spermatozoa and semen (Ali et al.; Li et al., 2012a, Biol Trace Elem Res, 2012, 148, 1-6; Marzec-Wroblewska et al.; Sorensen et al., 1999, Molecular human reproduction 5, 331-337; Wang et al., 2016; and Wang et al., 2017). Despite traditional ICP-MS technology has been applied in sperm elemental composition evaluation (see, for example, Sorensen et al, 1999), however, it cannot achieve elemental analysis at the single cell level without the advancement of recent technology (Cao et al., 2019, Talanta 206, 120174; Ho and Chan, 2010, J Anal Atom Spectrom 25, 1114-1122; Laborda et al., 2014, Anal Chem 86, 2270-2278; Liu et al., 2010, Talanta 83, 48-54; Meyer et al., 2018, Metallomics 10, 73-76; Miyashita et al.; Mueller et al., 2014, Anal Bioanal Chem 406, 6963-6977; and Wang et al., 2015, Analyst 140, 523-531). The latest development of single-cell inductively coupled plasma mass spectrometry (sc-ICP-MS) technology allows direct detection of the element content in a single cell. Several studies have applied sc-ICP-MS to quick and simultaneous evaluation of the composition of multiple elements at single particle or single mammal cell level (Bandura et al., 2009, Anal Chem 81, 6813-6822; Cao et al.; Ho and Chan; Meyer et al.; Miyashita et al.; Mueller et al.; and Wang et al., 2015). The sc-ICP-MS technology uses a single-cell nebulizer to atomize the cell suspension into small droplets containing single cells into the single-cell fog chamber, and realizes single-cell-level analysis through a rapid-analyzing computer software module. However, most of the validated cells are all somatic cells, including red blood cells and human cell lines, and there are no related detection methods for highly morphological polarized cells such as spermatozoa or cells isolated form body fluid. And thus far it has never applied in the analysis of single human spermatozoon.

As illustrated in the Examples, sc-ICP-MS technology was employed to verify the feasibility of elementomic characteristic analysis for the evaluation of normal human spermatozoa and oligoasthenozoospermia at the single cell level. Laboratory commonly used cultured somatic cells, such as cancerous cells, were also included in the analysis for comparison purpose. To this end, eighteen elements were measured using sc-ICP-MS in single cells of a given biological sample for validation. It was found that different cell types not only have different single cell elementomic profiles, but also have unique dynamic kinetics of sc-ICP-MS signal spike of specific elements. In highly polarized spermatozoa, with the exception of Ca, no difference was observed in the mean mass content of elements in single normal spermatozoon and oligoasthenozoospermia. However, further analysis on frequency distribution of the mean mass content and the element-to-element correlations revealed significant differences in certain elements measured in normal and abnormal sperm, such as the inverse correlation between the essential elements K and Na, as well as the toxic elements Cd or Pb inversely correlated with the essential element Ca. Moreover, by using more sophisticated analysis, the unique dynamic kinetics of single sc-ICP-MS spike signals of specific elements (such as Fe and Cu) can be determined in human spermatozoa. These elementomic characteristics of Fe and Cu in normal human sperm were significantly different from that of oligoasthenozoospermia under normal versus capacitated conditions. The elementomic characteristics of somatic cells, including cancer cells, were also found to be different from that of human spermatozoa. It was then proposed that this previously unreported single-cell elemental-specific kinetic characteristic in specific cell types, such as human spermatozoa, can be used as an elementomic signature evaluating physiological and pathophysiological status of specific cell types of a given biological sample from a subject. As a summary, the disclosure herein explores potential uses of single-cell ICP-MS elementomic characterization analysis for the diagnosis and prognosis of medical diseases and conditions, such as sperm functional quality, male fertility, and even cancer.

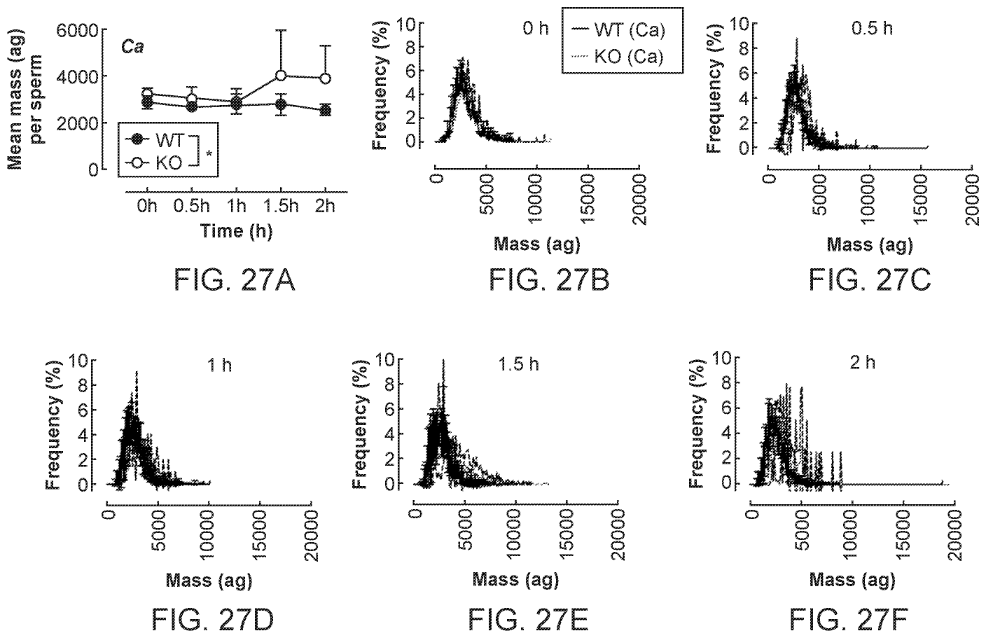

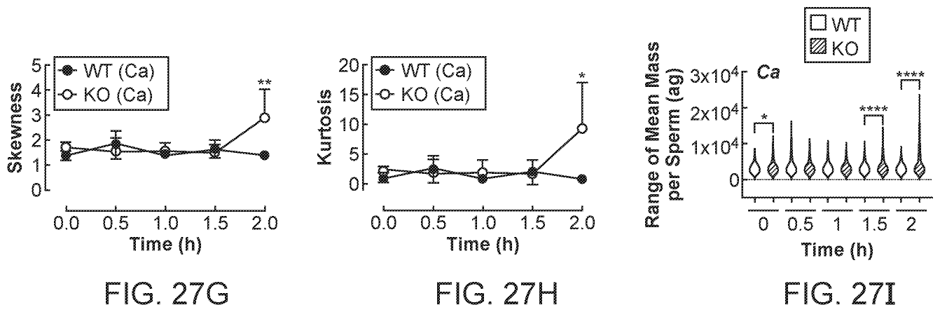

In some embodiments and as a proof-of-concept demonstration, employed herein is single-cell inductively coupled plasma mass spectrometry (ICP-MS) technology to measure the contents of inorganic metal ions in live single sperm cells undergoing the capacitation process. A male sterile calcium efflux pump Pmca4 KO mouse model was used, demonstrating, for the first time, the uses of a single-cell ICP-MS technology to assess sperm functional activity and to evaluate the elemental composition of single sperm cells during in vitro capacitation process to correlate the sperm quality of individual animal. Accordingly, the disclosure herein provides a new method and a metallomics-based diagnostic tool for the assessment of sperm quality and male fertility. Sterile Pmca4 KO sperm cells were used to monitor the dynamic intracellular calcium levels as well as the levels of other elements for example, but not limited to, Zn, Fe, Cu, Mn, and Se (FIG. 27A-27I to FIG. 33A-33F). This previously unreported method is useful for experimental and clinic evaluation of metal composition during dynamic biological process to identify normal and abnormal sperm metallomic characteristics at a single cell level.

The scientific discovery disclosed herein provides dynamic and kinetic characteristics of element-specific sc-ICP-MS signal of a specific cell, thereby solving the technical issues of assessing functions of the cell, for example under a clinical setting, utilizing multilateral analysis of single-cell ion spectroscopy. This technology can be widely used in the quality examination of specific cells, including but not limited to the following cells and their related functions:
(1) providing reference parameters for sperm function, infertility and offspring health;
(2) providing reference parameters for function and infertility of an egg cell; (3) providing reference parameters for function and infertility of granular cell; (4) providing reference parameters for function, physiological healthy status and pathological diagnosis of blood leukocytes;
(5) providing reference parameters for diagnosis of physiological healthy status and pathological conditions of cells isolated from urine;
(6) providing reference parameters for diagnosis of physiological healthy status and pathological conditions of cells (including somatic and prokaryotic cells) isolated from stool; or
(7) providing reference parameters for diagnosis of physiological healthy status and pathological conditions of cells isolated from saliva.

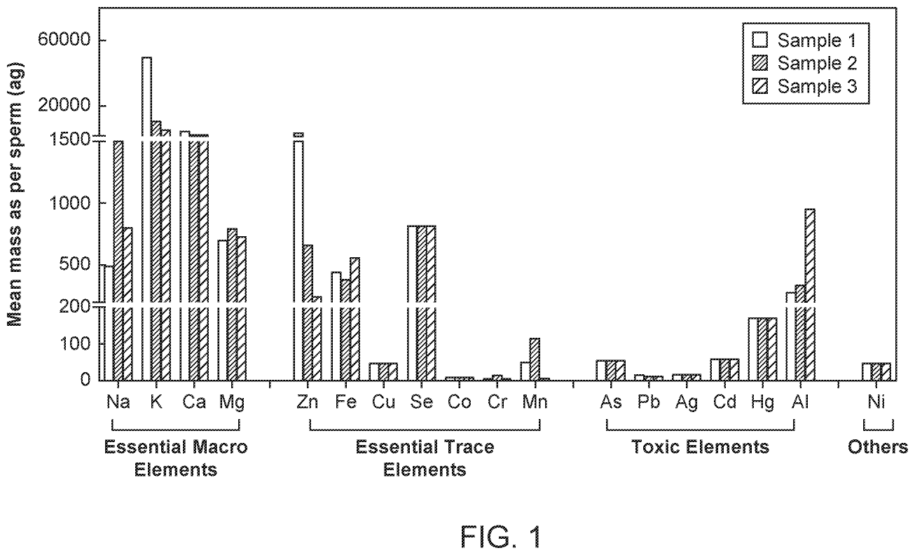

In some embodiments, the methods described in this disclosure is to detect multiple elements in trace amounts of single cells and to analyze dynamic or kinetic parameters of the element-specific ICP-MS signal spike in a particular cell type as well as the correlation of one element to other elements at the single cell level in the same biological samples to be detected. In some embodiments, the present disclosure provides a method for evaluating functional quality of a cell, comprising, or consisting essentially of, or yet further consisting of detecting bioavailability of specific elements in specific cell types, as well as the kinetic or dynamic characteristics of ICP-MS single signal spike of specific elements in specific cell types, such as dwell time, peak time of a single signal spike, its ratio to the dwell time, peak value of the elemental signal, as well as constants and area under the pre-peak and the post-peak of the signal spike. See, for example FIGS. 1-4 for the bioavailability and FIG. 5 for kinetic parameters.

In some embodiments, the purpose of the present disclosure is to solve the technical problems of how to determine the content of multiple elements in a trace amount of cells or even in a single cell, and how to evaluate the functional quality of the cell. In some embodiments, a method of the present disclosure can be applied to determine the composition of multiple elements in trace cells or in even single cells, and the determined composition can be used as a reference parameter for evaluating the functional quality of cells. In some embodiments, a method of the present disclosure can be used to analyze the signal dynamics characteristics of specific elements in a single cell, and the resultant characteristics can be used as a reference parameter for evaluating the functional quality of the cell. In some embodiments, the disclosure provides methods of evaluating human sperm function. The method comprises, or consists essentially of, or yet further consists of detecting multiple trace elements in trace amounts of single cells.

In some embodiments, a method as disclosed herein evaluates quality of a culture medium and measures contents (such as absolute amount or concentration) of essential elements and toxic heavy metals.

In some embodiments, the disclosure herein develops a software analyzing elemental bioavailability and dynamic or kinetic characteristics of elemental-specific ICP-MS signal spike in single cells. See, the Examples.

In some embodiments, a method of this disclosure is applicable to all morphologies and types of cells, greatly expanding the application range of sc-ICP-MS. In some embodiments, the present disclosure provides a method of detecting multiple trace elements in trace amounts of single cells and uses thereof in evaluating cells of heterogeneous morphologies and types. In some embodiments, the present disclosure provides a method of evaluating functional quality of a cell and uses thereof in evaluating cells of various morphologies and types. In some embodiments, the cells are of any species, such as all somatic cells, gamete cells, prokaryotic cells, or cells isolated from semen, fluids of reproductive tracts, follicular fluid, blood, urine, saliva, or faeces. In some embodiments, the present disclosure uses sperm in human semen as a model to test the feasibility of a method as disclosed herein, and then tests cells isolated from other human body fluids.

In some embodiments, the methods in this disclosure can be applied to the following levels.

(1) Cell level: for one example, a method as disclosed herein is used to detect the change of the element content in relation to the change of the element concentration in the culture medium by adding the trace elements in different concentrations to the cultured cells; and for an additional or alternative example, a protein known to bind to certain elements is overexpressed, knockdown, or knockout in the cells (for example, gamma-glutamyl carboxylase (GGCX), matrix Gla protein (MGP), occludin (OCLN), TRPV6, TMEM16A, lipocalins (LCNs), etc.), and a method as described in this disclosure is used to detect the element difference in the cell with or without addition of the element. Further, the results can be verified by detecting the changes of other elements.

(2) Animal level: for one example, using the animal model of infertility or subfertility with a known element homeostasis disorder, a method as disclosed herein is used to detect fertility of epididymal sperm and/or isolated epithelial cells from infertile animals or animals having a low fertility with the element homeostasis disorder; and for an additional or alternative example, the method as disclosed herein is (a) to determine the differences among various element characteristics of epididymal sperm from fertile animals, as well as changes in various element characteristics of sperm during different capacitation periods, (b) to determine the relationship of the various elements and a known element upon supplementing the various elements in medium culturing epididymal sperm from infertile animals or animals having a low fertility animal, or (c) to determine whether supplementing the various elements by oral or injection improves sperm quality optionally using CASA, IVF, ICSI, etc., or improves the fertility of the animal optionally by naturally mating the animals with healthy female.

(3) Clinical level: for one example, a method as disclosed herein is to analyze sperm collected from clinically healthy males and sperm collected from representative infertile males (such as patients having asthenozoospermia or, idiopathic infertility, or sperm after cryo-resuscitation, etc.); and for an additional or alternative example, a method as disclosed herein is (a) to establish the relationship of contents of various elements in sperm and seminal plasma, (b) to screen the various elements, compare the element composition in known sperm culture medium and adjust the concentration of various elements in the sperm culture medium thus improving the sperm culture medium, or (c) to screen the various element in sperm culture medium for an element or a concentration thereof improves sperm quality, optionally evaluated using CASA, IVF, ICSI, etc.

### Methods and Uses

In one aspect, provided is a method for detecting infertile spermatozoa in a sample. The method comprises, or consists essentially of, or yet further consists of detecting a concentration of at least one metal selected from the group of sodium (Na), potassium (K), calcium (Ca), magnesium (Mg), zinc (Zn), iron (Fe), copper (Cu), selenium (Se), cobalt (Co), chromium (Cr), cadmium (Cd), manganese (Mn), arsenic (As), mercury (Hg), lead (Pb), silver (Ag), aluminium (Al), and nickel (Ni) that falls outside of a predetermined range in spermatozoa using single-cell inductively coupled plasma mass spectrometry (sc-ICP-MS). In one aspect, the at least one metal is selected from sodium (Na), potassium (K), calcium (Ca), magnesium (Mg), zinc (Zn), iron (Fe), copper (Cu), selenium (Se), cobalt (Co), chromium (Cr), cadmium (Cd), manganese (Mn), arsenic (As), mercury (Hg), lead (Pb), silver (Ag), aluminium (Al), or nickel (Ni). In one aspect, the at least one metal is sodium (Na). In one aspect, the at least one metal is potassium (K). In one aspect, the at least one metal is magnesium (Mg). In one aspect, the at least one metal is zinc (Zn). In one aspect, the at least one metal is iron (Fe). In one aspect, the at least one metal is copper (Cu). In one aspect, the at least one metal is selenium (Se). In one aspect, the at least one metal is cobalt (Co). In one aspect, the at least one metal is chromium (Cr). In one aspect, the at least one metal is cadmium (Cd). In one aspect, the at least one metal is manganese (Mn). In one aspect, the at least one metal is arsenic (As). In one aspect, the at least one metal is mercury (Hg). In one aspect, the at least one metal is lead (Pb). In one aspect, the at least one metal is silver (Ag). In one aspect, the at least one metal is aluminium (Al). In one aspect, the at least one metal is nickel (Ni).

In some embodiments, the spermatozoa are capacitated. In some embodiments, the spermatozoa are not capacitated.

In some embodiments, the predetermined range corresponds to metal concentrations detected in spermatozoa from a population of fertile subjects.

In some embodiments, the predetermined range of the Na concentration is between about 5 attogram (ag) to about 50,000 ag for spermatozoa that are not capacitated and between about 25 ag to about 50,000 ag for spermatozoa that are capacitated. In some embodiments, the predetermined range of the K concentration is between about 50 ag to about 50,000 ag for spermatozoa that are not capacitated and between about 280 ag to about 50,000 ag for spermatozoa that are capacitated. In some embodiments, the predetermined range of the Ca concentration is between about 200 ag to about 50,000 ag for spermatozoa that are not capacitated and between about 700 ag to about 20,500 ag for spermatozoa that are capacitated. In some embodiments, the predetermined range of the Mg concentration is between about 8 ag to about 50000 ag for spermatozoa that are not capacitated and between about 75 ag to about 15,100 ag for spermatozoa that are capacitated. In some embodiments, the predetermined range of the Zn concentration is between about 5 ag to about 50,000 ag for spermatozoa that are not capacitated and between about 20 ag to about 50,000 ag for spermatozoa that are capacitated. In some embodiments, the predetermined range of the Fe concentration is between about 5 ag to about 50,000 ag for spermatozoa that are not capacitated and between about 13 ag to about 50,000 ag for spermatozoa that are capacitated. In some embodiments, the predetermined range of the Al concentration is between about 3 ag to about 50,000 ag for spermatozoa that are not capacitated and between about 6 ag to about 50000 ag (e.g. about 6 ag to about 46700 ag) for spermatozoa that are capacitated. In some embodiments, the predetermined range of the Se concentration is between about 59 ag to about 50,000 ag for spermatozoa that are not capacitated and between about 62 ag to about 45,810 ag for spermatozoa that are capacitated. In some embodiments, wherein the predetermined range of the Co concentration is between about 3 ag to about 3,700 ag for spermatozoa that are not capacitated and between about 9 ag to about 20,200 ag for spermatozoa that are capacitated. In some embodiments, the predetermined range of the Cu concentration is between about 9 ng to about 50,000 ag for spermatozoa that are not capacitated and between about 9 ng to about 37,590 ag for spermatozoa that are capacitated. In some embodiments, the predetermined range of the Cr concentration is between about 4 ng to about 50,000 ag for spermatozoa that are not capacitated and between about 5 ag to about 46,700 ag for spermatozoa that are capacitated. In some embodiments, the predetermined range of the Mn concentration is between about 2 ag to about 50,000 ag for spermatozoa that are not capacitated and between about 7 ag to about 32,610 ag for spermatozoa that are capacitated.

In some embodiments, the mean mass of As concentration corresponds to about 60 ag or less per cell per spermatozoon either capacitated or uncapacitated; the predetermined range of the mean mass of Ag concentration corresponds to about 800 ag or less per cell per spermatozoon either capacitated or uncapacitated; the predetermined range of the mean mass of Cd concentration corresponds to about 510 ag or less per cell per spermatozoon either capacitated or uncapacitated; the mean mass of Hg concentration corresponds to about 5400 ag or less per cell per spermatozoon either capacitated or uncapacitated; the predetermined range of the mean mass of Pb concentration corresponds to about 1610 ag or less per cell per spermatozoon either capacitated or uncapacitated; the mean mass of Ni concentration corresponds to about 2570 ag or less per cell per spermatozoon either capacitated or uncapacitated. As one of skill in the art would understand, a corresponding predetermined range can refer to the same ranges, but is detected or calculated using a parameter other than ag of the metal per spermatozoon, such as concentration of the metal in the spermatozoa. Accordingly, such corresponding predetermined ranges are also included in the disclosure herein.

In one aspect, provided is a method for detecting infertile spermatozoa in a sample. The method comprises, or consists essentially of, or yet further consists of: detecting a dynamic or kinetic parameter of a signal spike of at least one metal selected from the group of sodium (Na), potassium (K), calcium (Ca), magnesium (Mg), zinc (Zn), iron (Fe), copper (Cu), selenium (Se), cobalt (Co), chromium (Cr), cadmium (Cd), manganese (Mn), arsenic (As), mercury (Hg), lead (Pb), silver (Ag), aluminium (Al), and nickel (Ni) that falls outside of a predetermined range in spermatozoa using single-cell inductively coupled plasma mass spectrometry (sc-ICP-MS).

In some embodiments, the predetermined range corresponds to dynamic or kinetic parameters detected in spermatozoa from a population of fertile subjects.

In some embodiments, the dynamic or kinetic parameter of the spike are selected from: dwell time, pre-peak dwell time, post-peak dwell time, peak time, ratio between the peak time and the dwell time, raising tau constant before the peak, dynamic area before the peak, tailing tau constant after the peak, dynamic area after the peak, or any combination thereof. In some embodiments, the detected dynamic or kinetic parameter of the spike comprises, or consists essentially of, or yet further consists of any one or more of the following: (a) a dwell time of the Fe spike between about 1.4 to about 7.9 ms for spermatozoa that are not capacitated and a dwell time of the Fe spike between about 1.5 to about 6.7 ms for spermatozoa that are capacitated; (b) a tailing tau constant after the peak of the Fe spike about between about 0.18 to about 0.81 ms for spermatozoa that are not capacitated and a tailing tau constant after the peak of the Fe spike between about 0.18 to about 0.90 ms for spermatozoa that are capacitated; (c) a raising tau constant before the peak of the Fe spike at about −0.35 ms or lower for spermatozoa that are not capacitated and a raising tau constant before the peak of the Fe spike at about −0.80 ms for spermatozoa that are capacitated; (d) a dwell time of the Cu spike at about 1.5 ms or shorter for spermatozoa that are not capacitated and that are capacitated; (e) a raising tau constant before the peak of the Cu spike at about −0.2 ms or lower for spermatozoa that are not capacitated and a raising tau constant before the peak of the Cu spike at about −0.6 ms or lower for spermatozoa that are capacitated; (f) a tailing tau constant after the peak of the Cu spike at about 0.15 ms or lower for spermatozoa that are not capacitated and a tailing tau constant after the peak of the Cu spike at about 0.2 ms or lower for spermatozoa that are capacitated; (g) a dwell time of the Zn spike at about 2.1 ms or shorter for spermatozoa that are not capacitated and a dwell time of the Zn spike at about 1.2 ms or shorter for spermatozoa that are capacitated; (h) a raising tau constant before the peak of the Zn spike at about −0.25 ms or lower for spermatozoa that are not capacitated and a raising tau constant before the peak of the Zn spike at about −0.20 ms or lower for spermatozoa that are capacitated; (i) a tailing tau constant after the peak of the Zn spike at about 1.15 ms or lower for spermatozoa that are not capacitated and a tailing tau constant after the peak of the Zn spike at about 0.25 ms or lower for, spermatozoa that are capacitated; (j) a dwell time of the Cr spike at about 3.25 ms or shorter for spermatozoa that are not capacitated and a raising tau constant before the peak of the Cr spike at about −0.45 ms or lower for spermatozoa that are not capacitated; (k) a tailing tau constant after the peak of the Cr spike between about 0.2 to about 0.5 ms for spermatozoa that are not capacitated; (1) a dwell time of the Se spike at about 1.5 ms or shorter for spermatozoa that are not capacitated; (m) a raising tau constant before the peak of the Se spike at about −0.15 ms or lower for spermatozoa that are not capacitated; and (n) a tailing tau constant after the peak of the Se spike at about 0.25 ms or lower for spermatozoa that are not capacitated.

In one aspect, provided is a method for detecting infertile spermatozoa in a sample. The method comprises, or consists essentially of, or yet further consists of: (i) contacting a first population of the spermatozoa from the subject with a Human tubal fluid (HTF) buffer and optionally centrifuging the first population; and (ii) detecting a concentration of at least one metal selected from the group of potassium (K), calcium (Ca), magnesium (Mg), mercury (Hg), silver (Ag), and aluminium (Al) in the first population of spermatozoa post the contacting step that is comparable to or lower than concentration present in a second population of spermatozoa not contacted with a HTF buffer using single-cell inductively coupled plasma mass spectrometry (sc-ICP-MS); or (iii) detecting a concentration of selenium (Se) in the first population of spermatozoa post the contacting step that is comparable to or higher than concentration present in the second population of spermatozoa not contacted with a HTF buffer using the sc-ICP-MS; or (iv) both (ii) and (iii).

In some embodiments, the sample is obtained from a subject. In some embodiments, the subject has or is suspect of having idiopathic infertility, asthenozoospermia, oligozoospermia, or oligoasthenozoospermia.

In some embodiments, a method as disclosed herein further comprises treating the subject with an infertility therapy. In some embodiments, the infertility therapy comprises, or consists essentially of, or yet further consists of administering the element, a decreased level of which is detected in the sample of the subject compared to a control. In some embodiments, the concentration of at least one metal in the spermatozoa is lower that the predetermined range and the infertility therapy comprises treatment with the at least one metal. Additionally or alternatively, the infertility therapy comprises, or consists essentially of, or yet further consists of administering an agent, such as a small molecule or a protein, binding to the element, an increased level of which is detected in the sample of the subject compared to a control. In some embodiments, a concentration of the metal lower than the predetermined range is detected in the spermatozoa and the infertility therapy comprises the at least one metal. In some embodiments, a subject is treated with infertility therapy or an infertility procedure.

In some embodiments, two or more of the metals are detected.

In some embodiments, the sample is diluted with a buffer. In some embodiments, the sample is diluted with a buffer prior to detecting steps. In some embodiments, the spermatozoa is diluted to a concentration of 3×10⁶ spermatozoa/ml or less prior to the detection by the sc-ICP-MS. Additionally or alternatively, the spermatozoa is diluted to 10 times or more prior to the detection by the sc-ICP-MS. In some embodiments, the spermatozoa is diluted using any one of the following buffers: an HTF buffer, 4-(2-hydroxyethyl)-1-piperazineethanesulfonic acid (HEPES) buffer, a Phosphate Buffered Saline (PBS), a tris(hydroxymethyl)aminomethane (tris) buffer, or a Bis-tris methane (bis-tris) buffer.

In some embodiments, the spermatozoa is centrifuged to remove the seminal plasma prior to the dilution.

In some embodiments, the sample comprises, or consists essentially of, or yet further consists of semen. In further embodiments, the semen is liquefied; or fixed; or capacitated; or cryopreserved; or liquefied and fixed; or liquefied and capacitated; or liquefied and cryopreserved; or fixed and capacitated; or fixed and cryopreserved; or capacitated and cryopreserved; or liquefied, fixed and capacitated; or liquefied, fixed and cryopreserved; or liquefied, capacitated and cryopreserved; or liquefied, fixed, capacitated and cryopreserved.

In some embodiments, a method as disclosed herein further comprises performing a computer-aided sperm analysis (CASA) on the spermatozoa.

In some embodiments, a method as disclosed herein further comprises purifying the spermatozoa having (i) the at least one metal concentration, or (ii) the dynamic or kinetic parameter, or both (i) and (ii), that fall within the predetermined range to obtain functional spermatozoa.

In some embodiments, a method as disclosed herein further comprises fertilizing the purified functional spermatozoa with an egg, for example via in vitro fertilization (IVF) or intracytoplasmic sperm injection (ICSI).

In some embodiments, a method as disclosed herein detects all somatic cells, gamete cells, prokaryotic cells, regardless of its species.

In some embodiments, the detection concentration of cells is below 3×10⁶/ml.

In some embodiments, the detected elements are more than two.

In some embodiments, dynamic or kinetic parameters (also referred to herein as dynamic or kinetic characteristics or dynamic or kinetic features) of an sc-ICP-MS signal spike of an element in a cell, comprise, or consist essentially of, or yet further consist of any one of more of the following: dwell time, peak time of a single signal spike, ratio of the peak time to the dwell time, area of the peak, dynamic constant before the peak, area before the peak, dynamic constant after the peak, or area after the peak. See, for example, FIG. 5.

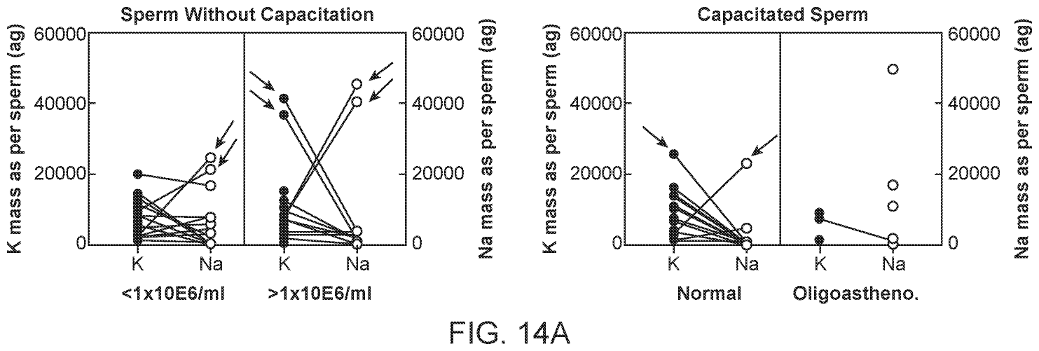

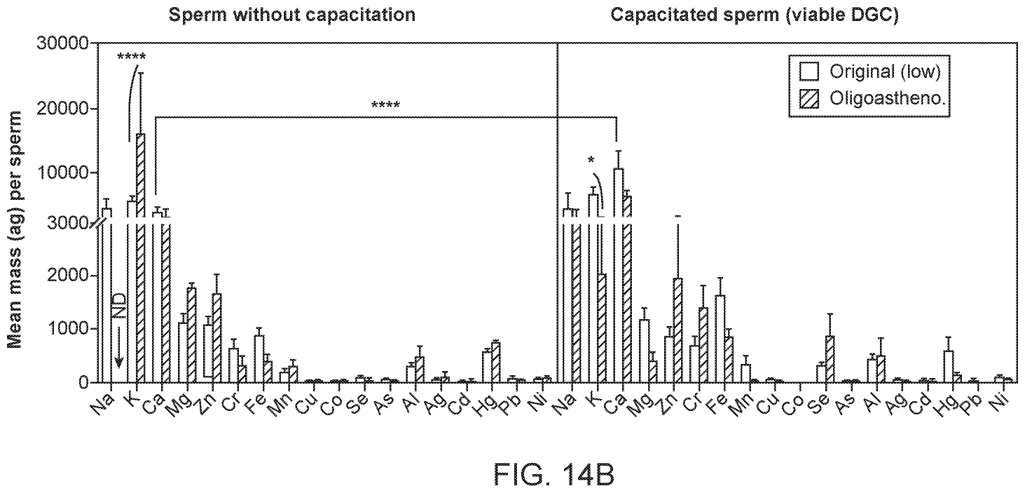

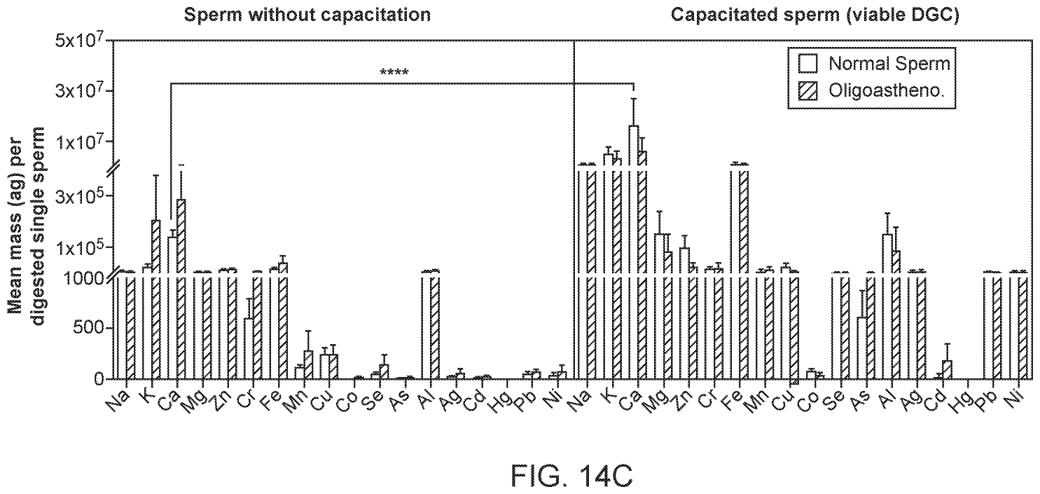

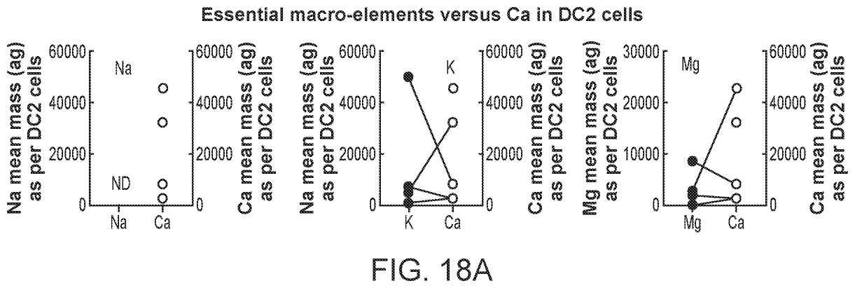

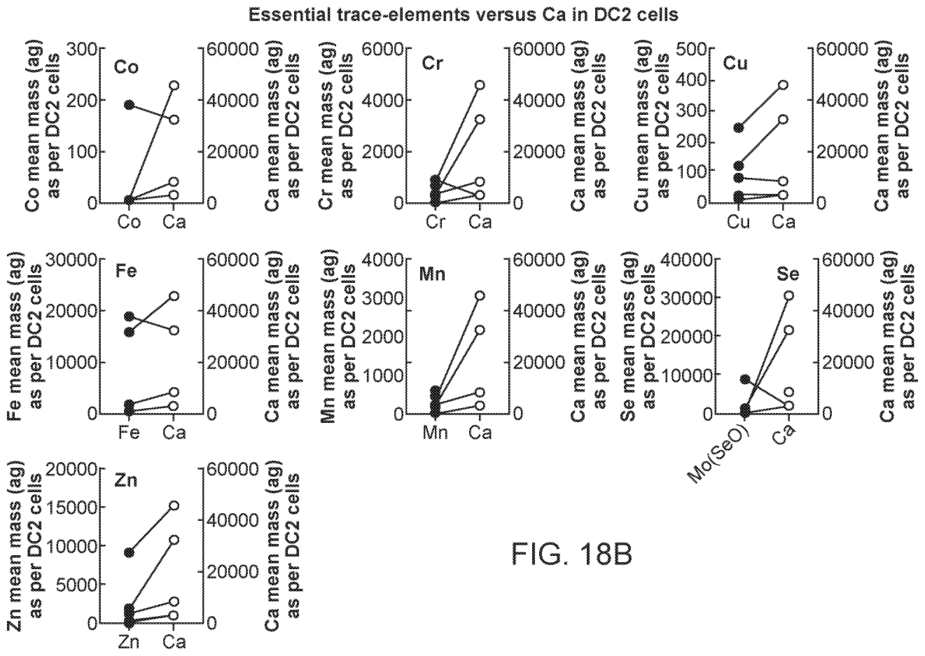

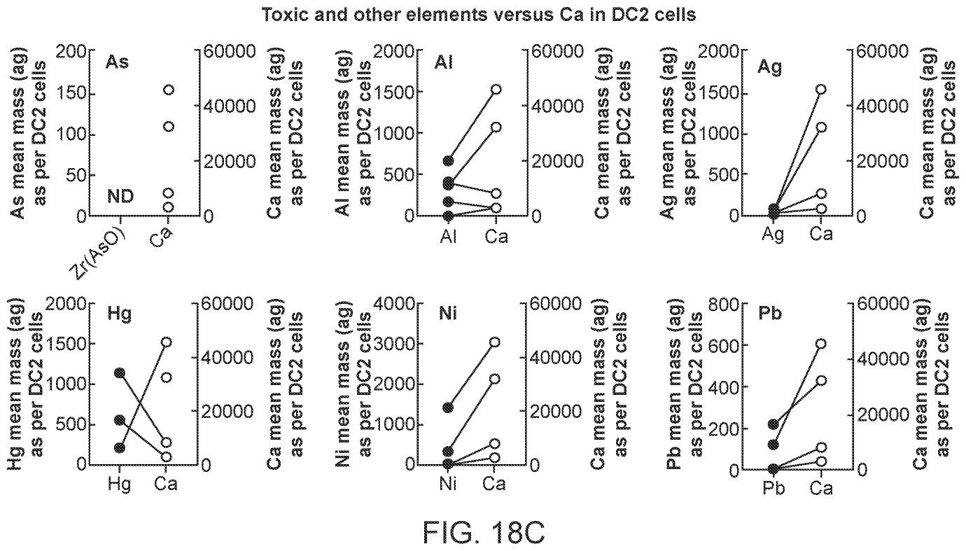

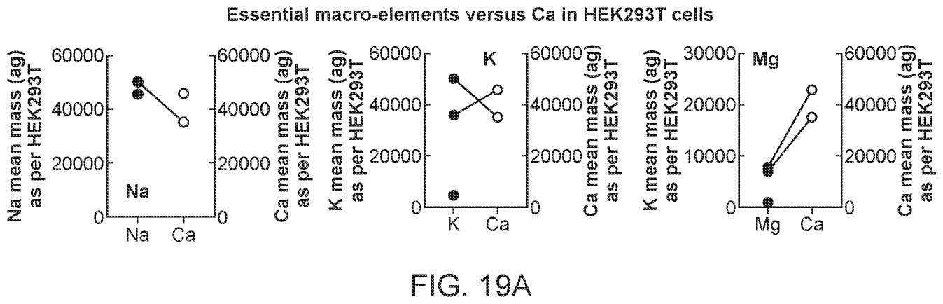

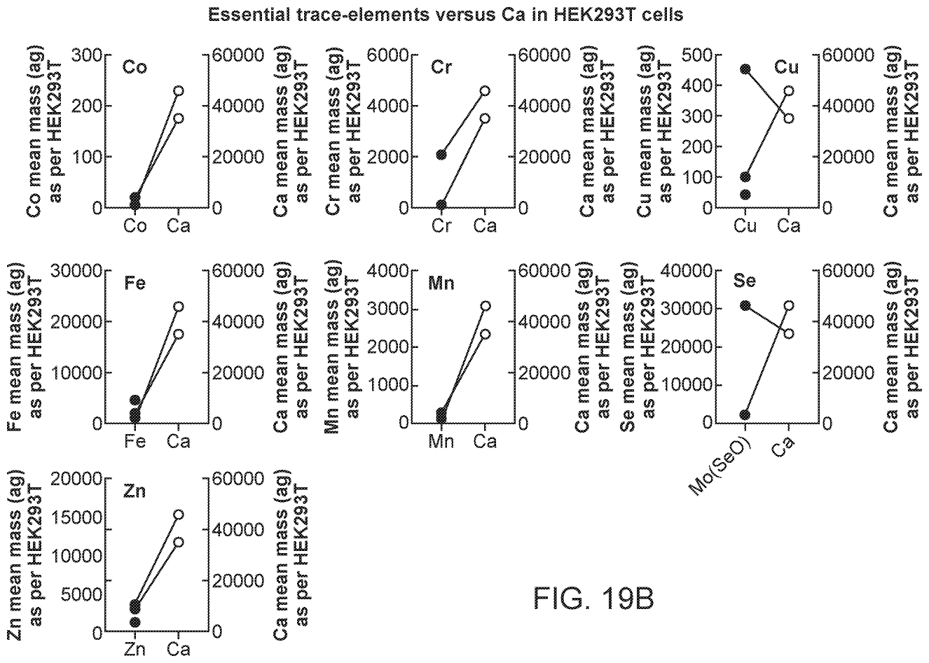

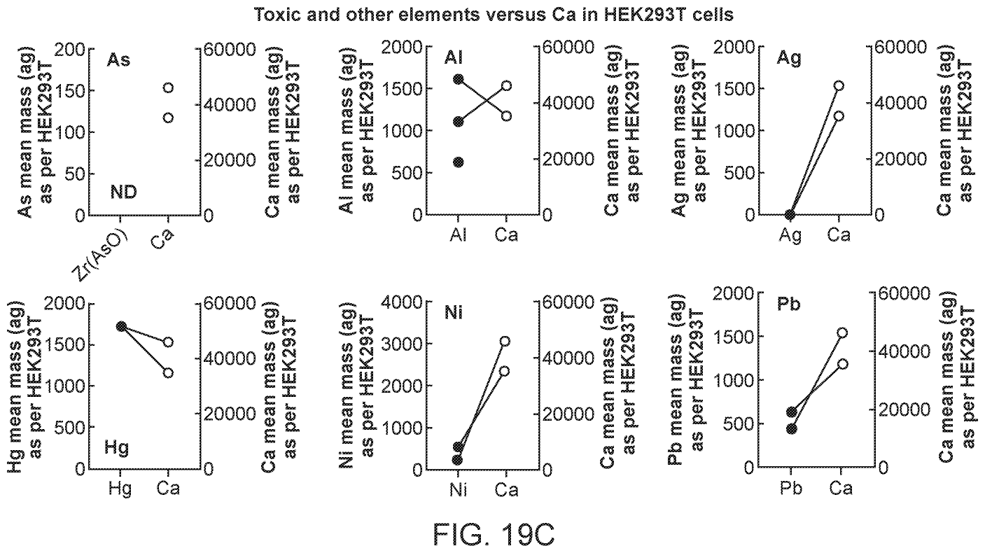

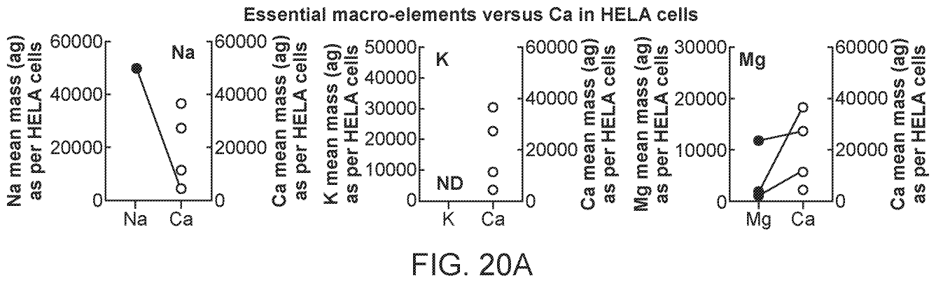

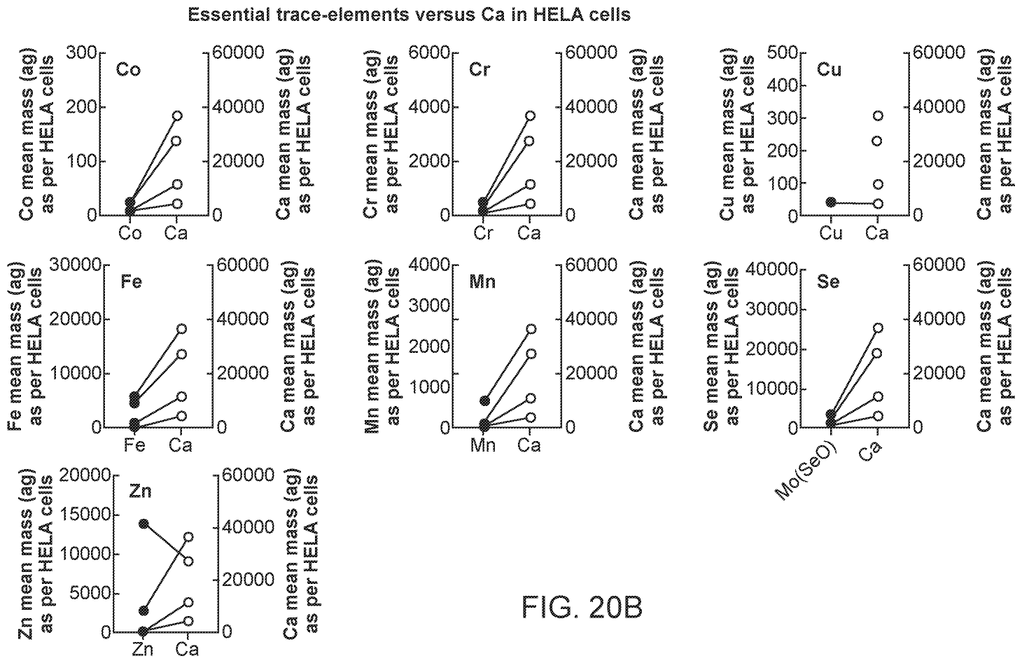

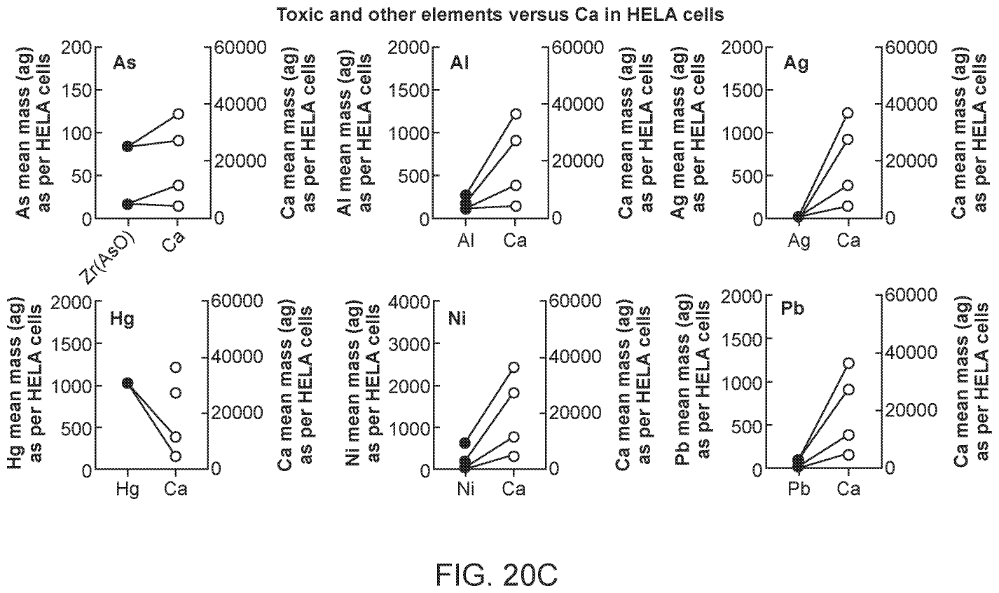

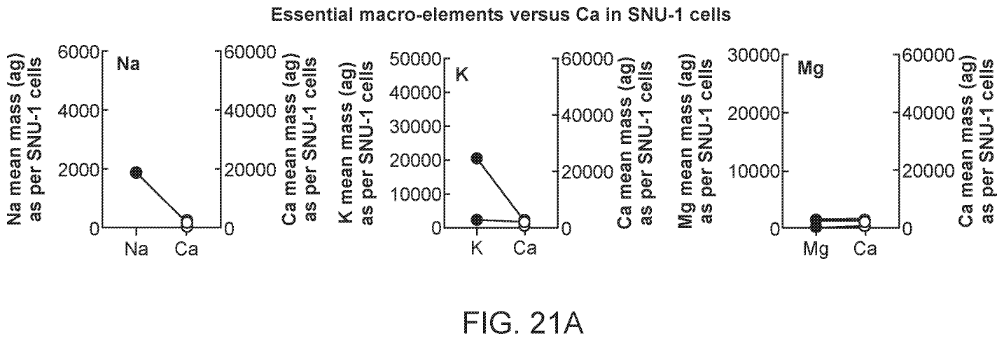

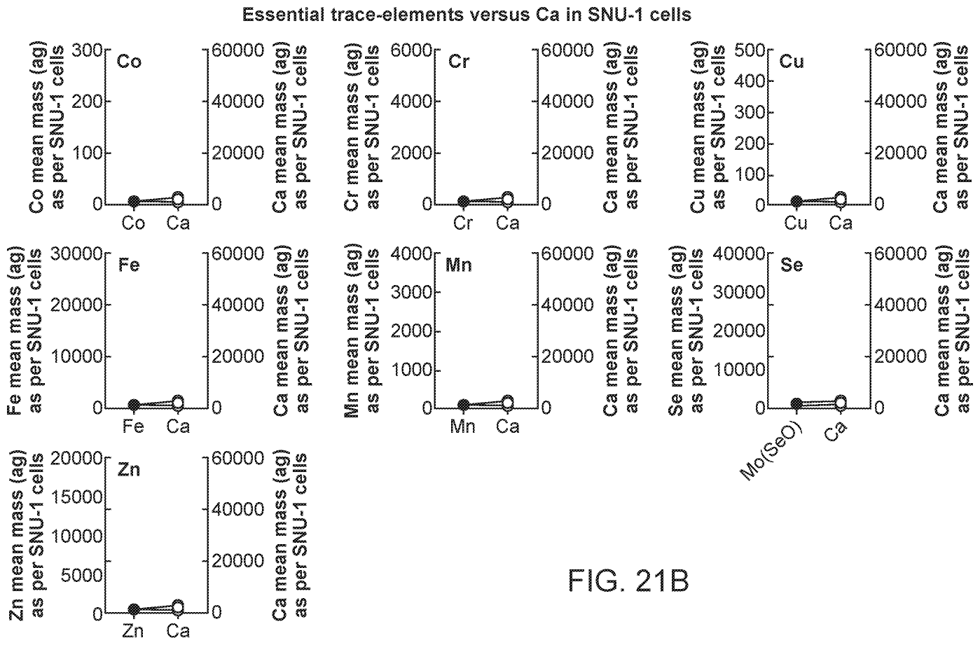

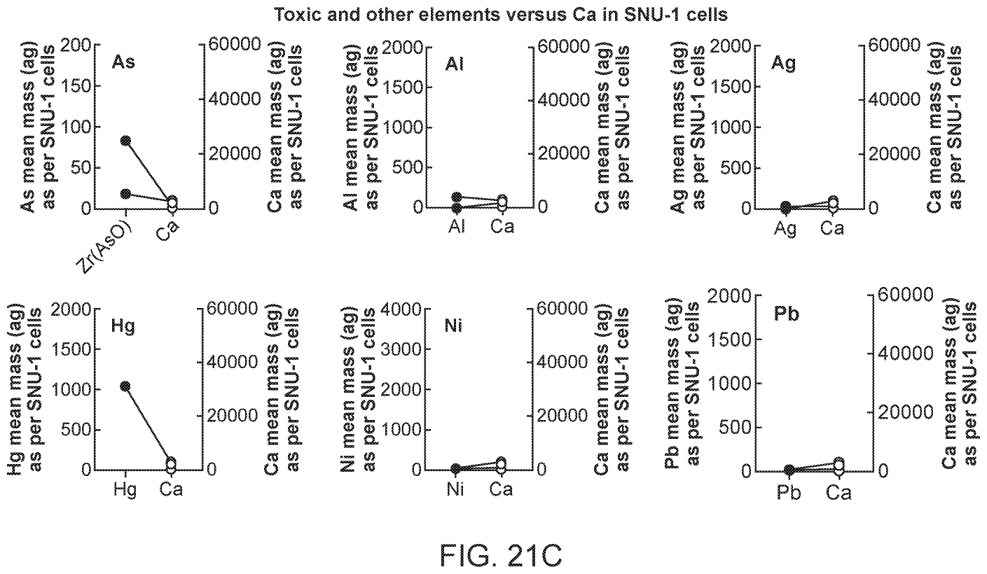

In some embodiments, the disclosure herein provides the correlation of bioavailability of an element with the calcium content at the single cell level of the same biological samples, for examples, the bioavailability of element with the calcium content in sperm cells (see, for example FIGS. 14-17), in wild-type somatic cells, in pathological somatic cells such as cancerous cells (see, for example, FIGS. 18-21).

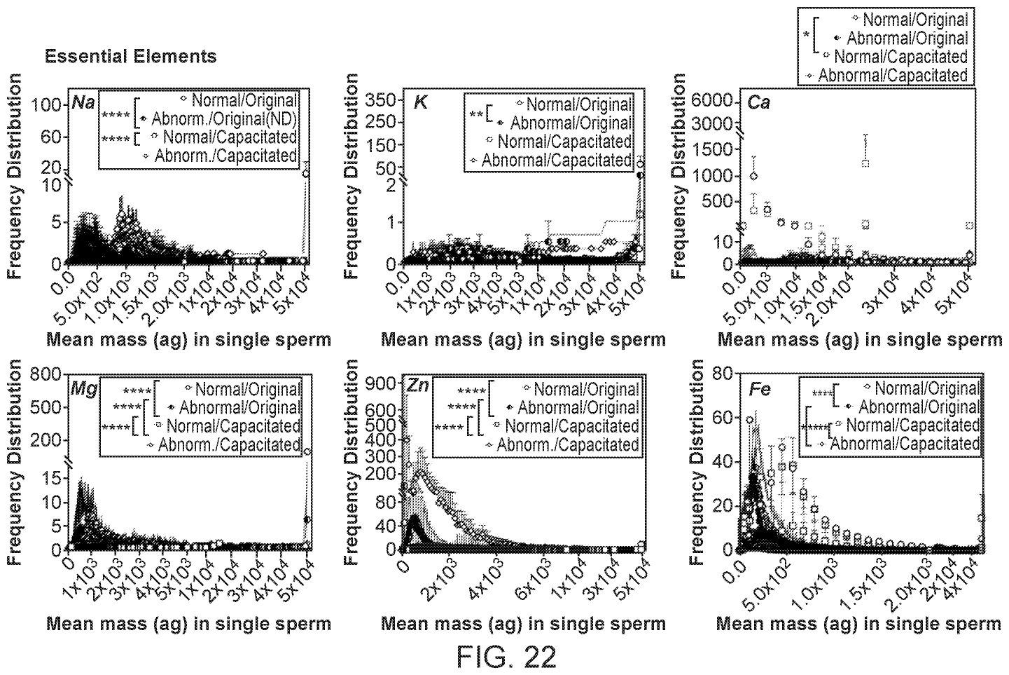

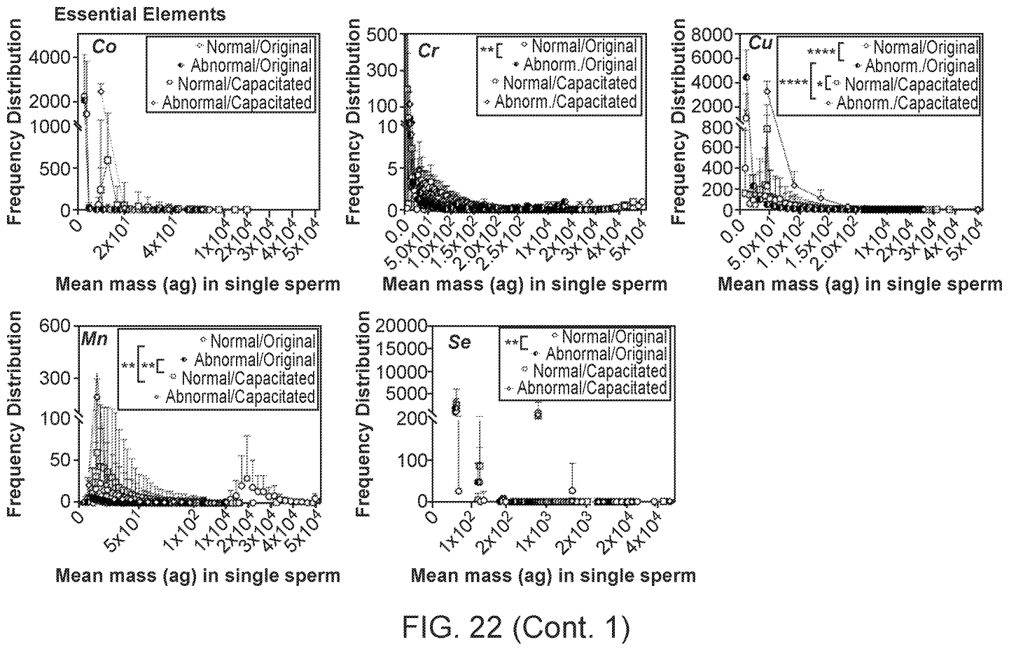

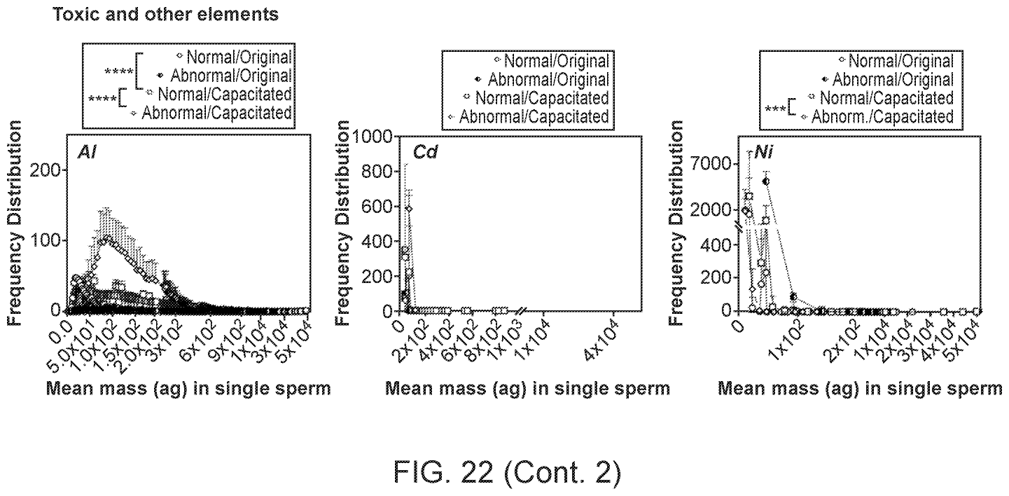

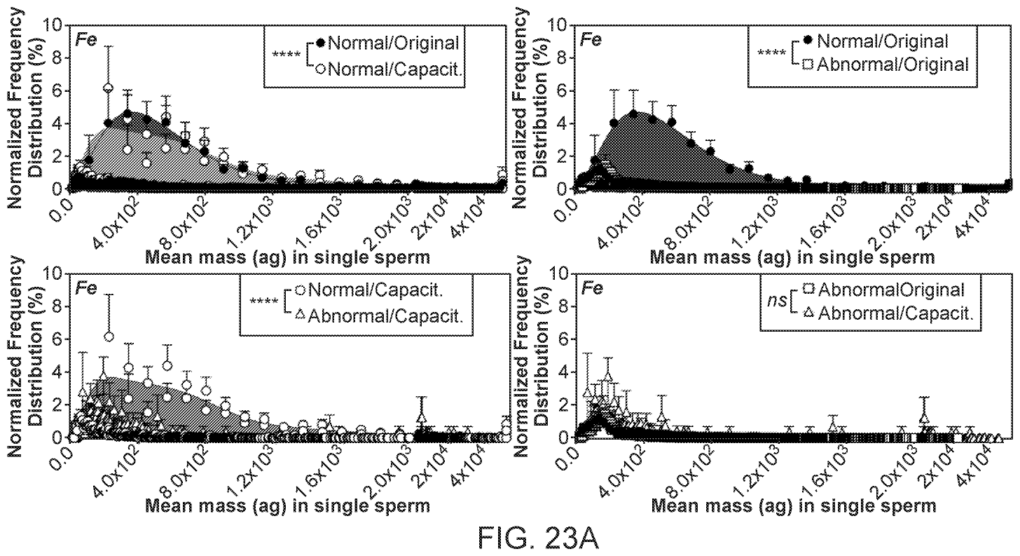

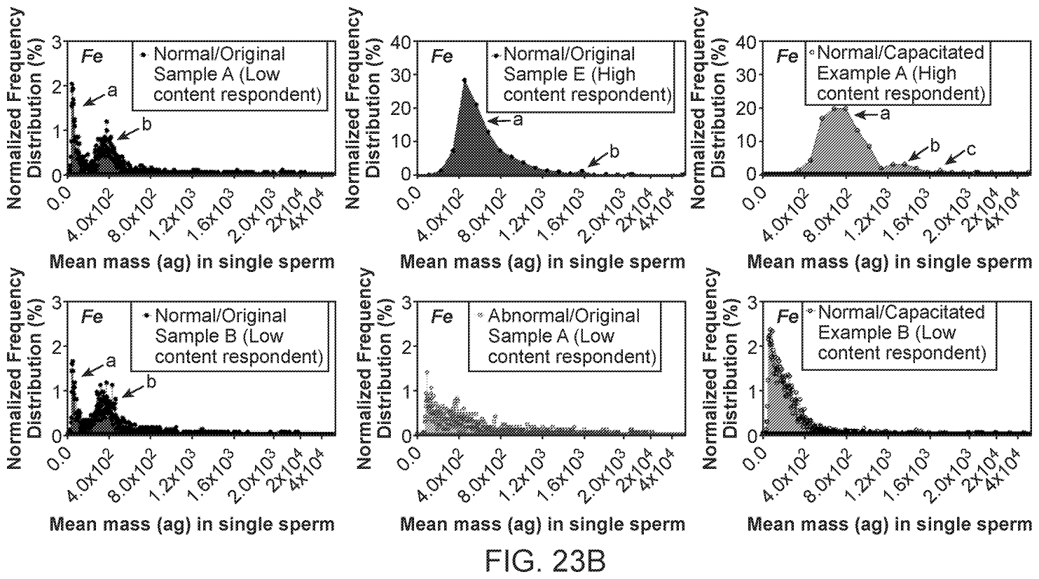

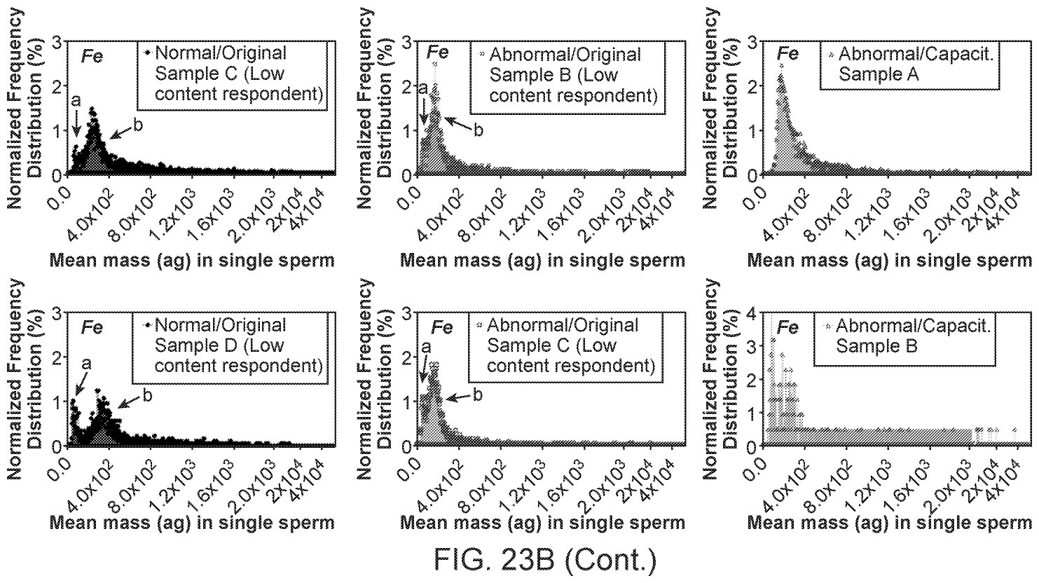

In some embodiments, the disclosure herein provides characteristics of frequency distribution of mean mass of the various elements detected in single human sperm cells of four physiological status (see, for example, FIG. 22) and the identification of sub-populations of essential elements in the single cells of the same biological samples, for examples, the bioavailability of element with the iron (Fe) content in sperm cells (see, for example, FIG. 23).

In some embodiments, unique single-cell elementomic profiles can be discovered in normal and abnormal (e.g. oligoasthenozoospermia) human spermatozoa.

In some embodiments, single-cell elementomic profiles of essential elements including Mg, Zn, Fe, Cu, Cr and Mn are associated with abnormal sperm functional risk, such as oligoasthenozoospermia risk;

In some embodiments, single-cell element-specific kinetic characteristics of ICP-MS signal in human spermatozoa are different from that of somatic cells, such as cancerous cells.

In some embodiments, the disclosure herein provides correlation of the single-cell element-specific kinetic characteristics of a specific element or in combination of two or more, in relation to the analytical outcome by applying Al (artificial intelligent) for matrix algorithm calculation, including the functional algorithms, for example, but not limited to, 1) Exponential alpha; 2) Exponential, cumulative probability; 3) Exponential, log probability; 4) Exponential, power; 5) Exponential, probability; 6) Exponential, product; 6) Exponential, sloping baseline; 7) Exponential, standard; 8) Exponential, weighted; 9) Exponential, weighted/constrained; 10) Guassian; 11) Binomial; 12) Polynomial; 13) Boltzman, charge-voltage; 14) Boltzman, shifted; 15) Boltzman, standard; 16) Boltzman, Z-delta (ascending); 17) Boltzman, Z-delta (descending), 18) Skewness; 19) Kurtosis; 20) Nonlinear regression (curve fit); 21) Simple linear regression; 22) Simple logistic regression; 23) Fit spline/Lowess; 24) Smooth, differentiate or integrate curve; 25) Area under curve; 26) Interpolate a standard curve; 27) t tests (and nonparametric tests); 28) One-way ANOVA (and nonparameteric or mixed model); 29) One sample t and Wilconxon text; 30) Frequency distribution; 31) Identify outliners; 32) Bland-Altman method comparison; 33) Two-way ANOVA (or mixed model); 34) Three-way ANOVA (or mixed model); 35) Multiple t tests (and nonparameteric tests); 36) Chi-square (and Fisher's exact) test; 36) Fraction of Total; 37) Correlation matrix; 38) Multiple linear regression; 39) Multiple logistic regression; 39) Principle component analysis; 40) Nested t-test; 41) Nested one-way ANOVA; etc.

In some embodiments, the disclosure herein provides correlation of the single-cell element-specific kinetic characteristics with health conditions, such as sperm functional quality or metabolic syndromes and related health conditions of a subject.

In some embodiments, the disclosure relates to a method and use of detecting multiple trace elements in trace cells, and belongs to the technical field of medical detection. The disclosure includes two parts: detection of multiple trace elements and signal kinetic analysis. Conventional single-cell inductively coupled plasma mass spectrometry (sc-ICP-MS) methods can be used: a single-cell nebulizer is used to aspirate the cell suspension into small droplets, each of which containing a single cell, into a single-cell spray chamber; bioavailability of elements and their kinetic characteristics of ICP-MS signal spike is analyzed at a single-cell level using a fast-digitizing software module; and the same method is used to prepare and detect the standards. Then the kinetic characteristics of specific elements in specific cell types are analyzed. The present disclosure can be applied to all somatic cells, gamete cells, prokaryotic cells, and human cancer cells and determines contents of multiple elements in trace amounts of the cells or even at a single cell level, thereby providing a reference parameter for evaluating the functional quality of cells. In some embodiments, the cells include but are not limited to cells isolated from semen, reproductive tract fluid, follicular fluid, blood, urine, saliva and feces.

The presently disclosed subject matter is directed to methods of aiding diagnosis, prognosis, monitoring and evaluation of a disease or other medical condition in a subject by detecting to determine the elemental availability (content of more than two elements) in trace amounts of single cells (e.g. sperm or somatic cells at cell density or concentration lower than 1×10⁶/ml) isolated from a biological sample from the subject. This method is to detect multiple trace and essential elements in trace cells and analyze the signal dynamics and kinetics of the said elements of the samples. The main steps of the multiple trace element detection part of the present disclosure include obtaining the cell-containing liquid and centrifuging the supernatant, with or without fixing, diluting with the diluent to the detection concentration below 1×10⁶/ml, load into the machine for detection, and signal characterization and data analysis in a single sample. The types of detected elements are more than two elements per measurement. Moreover, disclosed subject is directed to methods of diagnosis, monitoring a disease by determining the profile of elemental bioavailability within a biological sample; methods of associating an element or its particular ratio relation to another element single cells all by profiling elements that associated said element or its particular ratio relation of other elements' concentration in the same measurement; methods for evaluation performing remedy for sperm functions of a subject; or method for evaluation of physiological or pathological status or both status, such as cancerous status, of single cells of a subject.

In some embodiments, a method as disclosed herein comprises, or consists essentially of, or yet further consists of obtaining somatic cells by centrifuging a sample, removing the supernatant, diluting the cells with a diluent to a detection concentration of below 3×10⁶/ml, loading the diluted cells into a conventional single-cell ICP-MS (sc-ICP-MS) instrument for detection. In some embodiments, the detection method uses the conventional single-cell ICP-MS (sc-ICP-MS): using a nebulizer to aspirate the cell suspension into small droplets, each of which containing a single cell, into the single-cell spraying chamber; using the same procedures to detect standards and generate standard curves of the elements; and detecting the elemental spike signals of each single cell in the small droplet of using a fast-analyzing digitizing software as provided by the manufacturer of the sc-ICP-MS machine and analyzing bioavailability of multiple trace elements and the element-specific spike signal characteristic analysis at the single cell level.

In some embodiments, in order to solve an issue as discussed herein, the disclosure herein is to provide a method for detecting multiple trace elements in trace amount of cells. The method comprises, or consists essentially of, or yet further consists of the following steps:
Step 1: obtaining somatic cells, centrifuging the cells, removing the supernatant, suspending the centrifuged cells with a diluent to a detection concentration of lower than 3×10⁶/ml, and loading the suspended cells into an sc-ICP-MS instrument for detection;
Step 2: performing the detection method using a conventional sc-ICP-MS method: aspirating the cell suspension using a single-cell nebulizer into droplets in a single-cell spray chamber, wherein each of the droplets comprises a single cell, and analyzing multiple trace elements at the single cell level using a fast-analyzing digitizing software module;
Step 3: detecting a series of standard solutions having known concentrations of the elements using the same procedure in step 2, and plotting a standard curve.
Step 4: determining the unique elemental ICP-MS signal characteristics of specific cell types using a method comprising evaluating the characteristics of sc-ICP-MS signal dynamics of specific elements of specific cells, selected from dwell time, peak time of a single signal spike, the time ratio of pre- or post-peak of spike in relation to the dwell time, dynamic kinetic constant or area of pre-peak of spike, dynamic constant or area of post-peak of spike, etc;
Step 5: assessing the functional quality of the cells.

In some embodiments, the present disclosure provides a method for detecting multiple trace elements in human sperm cells. The method comprises, or consists essentially of, or yet further consists of the following steps:
Step 1: sample preparation: liquefying fresh human semen according to the conventional semen preparation method, centrifuging the liquefied semen, separating seminal plasma and sperm, re-suspending the sperm in 4% PFA, fixing the sperm in 4% PFA for 15 minutes, removing the fixing solution by washing, and performing the following steps using the sperm or storing the sperm at four degree Celsius for later use;
Step 2: diluting the sperm with a diluent to a detection concentration of lower than 3×10⁶/ml;
Step 3: loading samples into an sc-ICP-MS instrument for detection: installing a specific single-cell nebulizer and spray chamber, tuning with a tuning solution (2% v/v nitric acid containing 10 μg/L Li, Be, Mg, Fe, In, Ce, Pb and U) to determine appropriate instrument detection parameters, using gold particle standards to determine the single cell transmission efficiency which is generally in the range of 40%-60%, preparing standard solutions of different concentrations of different elements, plotting standard curve, wherein all standard solutions are prepared in ultrapure water, wherein in order to eliminate polyatomic interference and to obtain a high signal-to-noise ratio, measurements are performed in the dynamic reaction cell (DRC) mode, using ammonia as the reaction gas for the detection of K, Ca, Cr, and Fe in single cells, or using oxygen as the reaction gas for the detection of As and Se in single cells by detecting the oxidation reaction products of AsO and SeO as the analysis species, wherein other elements are measured in standard mode, wherein the sample spike signals are sampled for 50 s with a dwell time of 50 μs (i.e. digitizing frequency at 50 MHz), and wherein the total sample volume consumed by detecting 18 elements is about 400 μL.
Step 4: determining the unique elemental ICP-MS signal characteristics of specific cell types using a method comprising evaluating the characteristics of sc-ICP-MS signal dynamics of specific elements of specific cells, selected from dwell time, peak time of a single signal spike, the time ratio of pre- or post-peak of spike in relation to the dwell time, dynamic kinetic constant or area of pre-peak of spike, and dynamic constant or area of post-peak of spike;
Step 5: assessing the functional quality of the cells.

### Kits

Additionally provided is a kit comprising, or consisting essentially of, or yet further consisting of buffer and instructions for performing a method as disclosed herein.

In some embodiments, the kit comprises a sterile container which contains the infertility therapy or the buffer or both; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

In some embodiments, the instructions generally include information about the use of the buffer and optional infertility therapy for a method as disclosed herein. In other embodiments, the instructions include at least one of the following: description of the buffer; description of the optional infertility therapy; dosage schedule and administration of the optional infertility therapy; precautions; warnings; indications; counter-indications; overdose information; adverse reactions; animal pharmacology; clinical studies; or references. The instructions may be printed directly on the container (when present), or as a label applied to the container, or as a separate sheet, pamphlet, card, or folder supplied in or with the container.

A device capable of delivering the kit components through an administrative route may be included. Examples of such devices include syringes (for parenteral administration) or inhalation devices. In some embodiments, the infertility therapy of the present technology may be provided in the form of a prefilled syringe or autoinjection pen containing a sterile, liquid formulation or lyophilized preparation (e.g., Kivitz et al., 2006, Clin. Ther. 28:1619-29).

The kit components may be packaged together or separated into two or more containers. In some embodiments, the containers may be vials that contain sterile, lyophilized formulations of an infertility therapy that are suitable for reconstitution. A kit may also contain one or more buffers suitable for reconstitution or dilution or both reconstitution and dilution of other reagents. Other containers that may be used include, but are not limited to, a pouch, tray, box, tube, or the like. Kit components may be packaged and maintained sterilely within the containers.

The following examples are included to demonstrate some embodiments of the disclosure. However, those of skill in the art should, in light of the present disclosure, appreciate that many changes can be made in the specific embodiments which are disclosed and still obtain a like or similar result without departing from the spirit and scope of the invention.

### EXAMPLES

### Example 1: Method and Application of Detecting Multiple Trace Elements in Trace Amount of Cells

In order to make the present disclosure more comprehensible, some embodiments and accompanying drawings are described in detail as follows: the present disclosure provides a method for detecting multiple trace elements in trace amounts of cells, which includes the following steps:
Step 1: obtaining gamete cells and/or somatic cells, centrifuging the cells, removing the supernatant, suspending the centrifuged cells with a diluent to a detection concentration of lower than 3×10⁶/ml, and loading the suspended cells into an sc-ICP-MS instrument for detection;
Step 2: performing the detection method using a conventional sc-ICP-MS method: aspirating the cell suspension using a single-cell nebulizer into droplets in a single-cell spray chamber, wherein each of the droplets comprised a single cell, and analyzing multiple trace elements at the single cell level using a fast-analyzing digitizing software module;
Step 3: detecting a series of standard solutions having known concentrations of the elements using the same procedure in step 2, and plotting a standard curve.
Step 4: determining the unique elemental ICP-MS signal characteristics of specific cell types using a method comprising evaluating characteristics of sc-ICP-MS signal dynamics of specific elements of the specific cells, selected from dwell time, peak time of a single signal spike, time ratio of pre- or post-peak of the spike in relation to the dwell time, dynamic kinetic constant or area of the pre-peak of the spike, dynamic constant or area of the post-peak of the spike, etc;
Step 5: assessing the functional quality of the cells.

As shown in FIGS. 1-5, provided is a method for detecting multiple trace elements in trace amounts of cells for use in evaluating human sperm cells. The method comprises, or consists essentially of, or yet further consists of
Step 1: sample preparation: liquefying fresh human semen according to the conventional semen preparation method, centrifuging the liquefied semen, separating seminal plasma and sperm, re-suspending the sperm in 4% paraformaldehyde (PFA), fixing the sperm in 4% PFA for 15 minutes, removing the fixing solution by washing, and performing the following steps using the fixed sperm or storing the fixed sperm at four degree Celsius for later use;
Step 2: diluting the sperm with a diluent to a detection concentration of lower than 3×10⁶/ml;
Step 3: loading samples into an sc-ICP-MS instrument for detection: installing a specific single-cell nebulizer and spray chamber, tuning with a tuning solution (2% v/v nitric acid containing 10 μg/L Li, Be, Mg, Fe, In, Ce, Pb and U) to determine appropriate instrument detection parameters, using gold particle standards (such as 50 nm gold particles) to determine the single cell transmission efficiency which was generally in the range of 40%-60%, preparing standard solutions having different concentrations of different elements (such as Na, K, Ca, Mg, Zn, Fe, Cu, Se, Co, Cr, Cd, Mn, As, Hg, Pb, Ag, Al, Ni, etc.), plotting standard curves, wherein the concentrations of the standard solutions of Hg were 0.5 part per billion (ppb), 1 ppb, and 2 ppb, wherein the concentrations of the standard solutions of Ca were 50 ppb, 100 ppb, and 200 ppb, wherein the concentrations of the standard solutions of other elements were 5 ppb, 10 ppb, and 20 ppb, wherein all standard solutions were prepared in ultrapure water, wherein in order to eliminate polyatomic interference and to obtain a high signal-to-noise ratio, measurements were performed in the dynamic reaction cell (DRC) mode, using ammonia as the reaction gas for the detection of K, Ca, Cr, and Fe in single cells, or using oxygen as the reaction gas for the detection of As and Se in single cells by detecting the oxidation reaction products of AsO and SeO as the analysis species, wherein other elements were measured in standard mode, wherein the sample spike signals were sampled for 50 s with a dwell time of 50 μs (i.e. digitizing frequency at 50 MHz), and wherein the total sample volume consumed by detecting 18 elements was about 400 μL. The normal working conditions of sc-ICP-MS are shown in Table 1 below.

TABLE 1

| Instrumental | Single Cell ICP-MS | |
|---|---|---|
| Parameters | Analysis | |
| ICP parameters | | |
| Nebulizer | Meinhard HEN Cyto | |
| | Nebulizer or equivalent | |
| Spray chamber | Asperon ™ Spray Chamber | |
| | or equivalent | |
| ICP RF power | 400 W-1600 W | |
| Plasma gas flow | 15 | L/min |
| Auxiliary gas flow | 1.2 | L/min |
| Nebulizer gas flow | 0.38 | mL/min |
| Sample Flow Rate | 0.016-0.025 | mL/min |
| Torch | Fixed 2.0 mm Injector | |
| | Quartz Torch | |
| Cones | Nickel, Tri-cone Design | |
| Measurement | | |
| parameters | | |
| Data acquisition | Standard or TRA | |
| mode | | |
| TRA duration | | |
| time (s) | | |
| Dwell Time | 50 | μs |
| Scan Time | 50 | s |
| Transport Efficiency | 39-60% | |
| DRC parameters | | |

| | |
|---|---|
| RPa | 0 |
| RPq | 0.45 ml/min for ⁵⁶Fe⁺, ⁵²Cr⁺, ⁴⁴Ca⁺, ³⁹K⁺, |
| | ⁹⁶Mo(SeO)⁺⁹¹Zr(AsO)⁺, |
| | 0.25 ml/min for others |
| DRC gas (NH₃) | 0.8 ml/min for ⁴⁴Ca⁺, |
| | 0.6 ml/min for ⁵²Cr⁺ and ⁵⁶Fe⁺, |
| | 1 ml/min for ³⁹K⁺ |
| DRC gas (O₂) | 0.45 ml/min for ⁹⁶Mo(SeO)⁺ |
| | and ⁹¹Zr(AsO)⁺ |

There is still a lack of methods for single sperm ion spectrum analysis in the clinics. Additionally, the ion content in sperm is also an important factor to evaluate sperm quality. Thus, it is important to establish a method to detect the ion spectrum of individual sperm, and to provide a diagnostic method for assessing the quality of sperm in clinical labs and cell function, thereby providing references and values in this regard. Further, unique characteristics of ICP-MS signals of specific elements can be distinguishable in single sperm cells, including the dwell time of different elements of single sperm, the peak time of a single signal and its ratio to the dwell time, the kinetic constant and area before the peak, and the kinetic constant and area after the peak. The characteristics of these ICP-MS signal dynamic kinetics have important biological significance and far-reaching implication relevant to pathological status evaluation, and thus have clinical application implications.

As shown in FIG. 1, quantitative analysis is provided of multiple elements in a single human sperm in different samples. By using the detection method as disclosed herein, the difference of multiple elements in different samples was detected. The lowest sensitivity for detection of multiple elements reached a concentration of atto-gram (ag) per cell (see, FIG. 1).

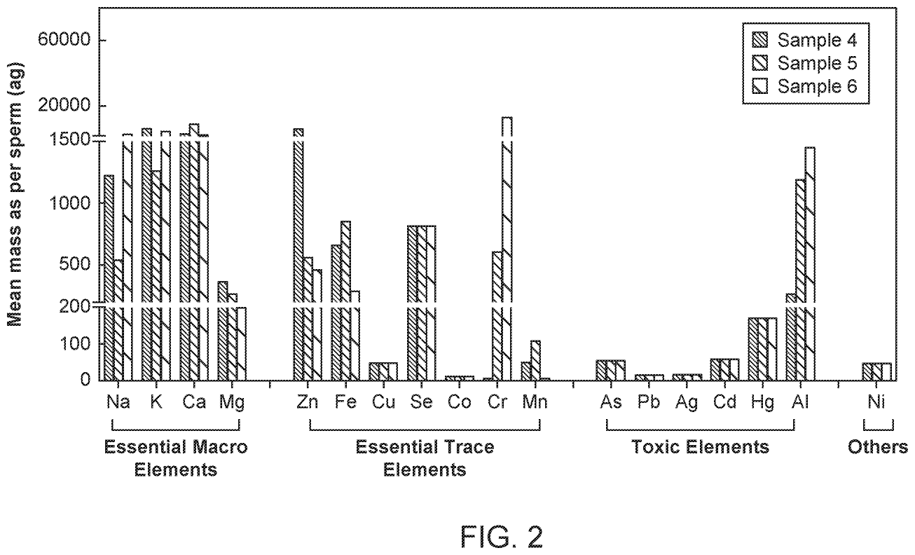

As shown in FIG. 2, an example is provided of a few samples with a relatively high content of essential trace elements in single human sperm. The detection method as disclosed herein detected the difference in the content of essential trace elements in different samples. These findings can be used as standards and suggestions for element supplementation can be made to subjects with the deficiency of these elements.

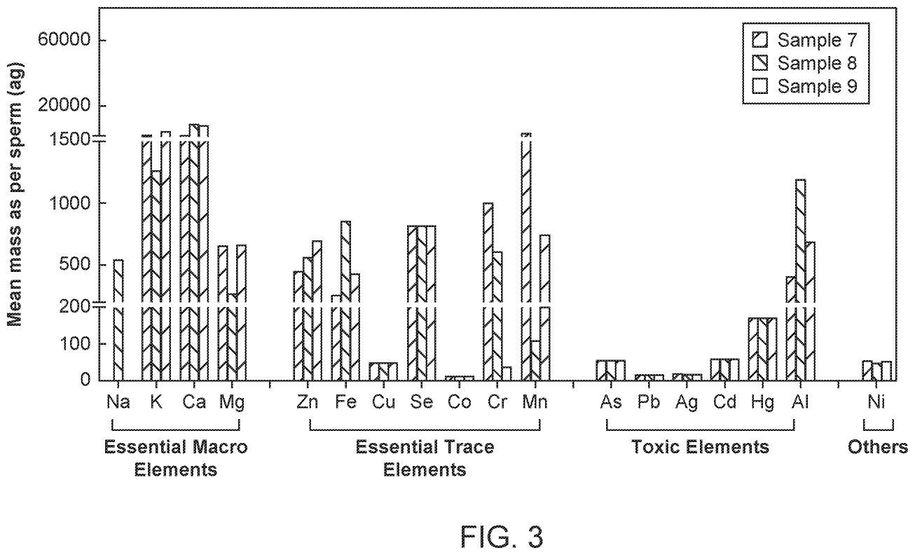

As shown in FIG. 3, an example is provided of a few samples with a relatively high content of toxic elements in single human sperm. These subjects with relatively high levels of toxic elements can be treated for clinical detoxification.

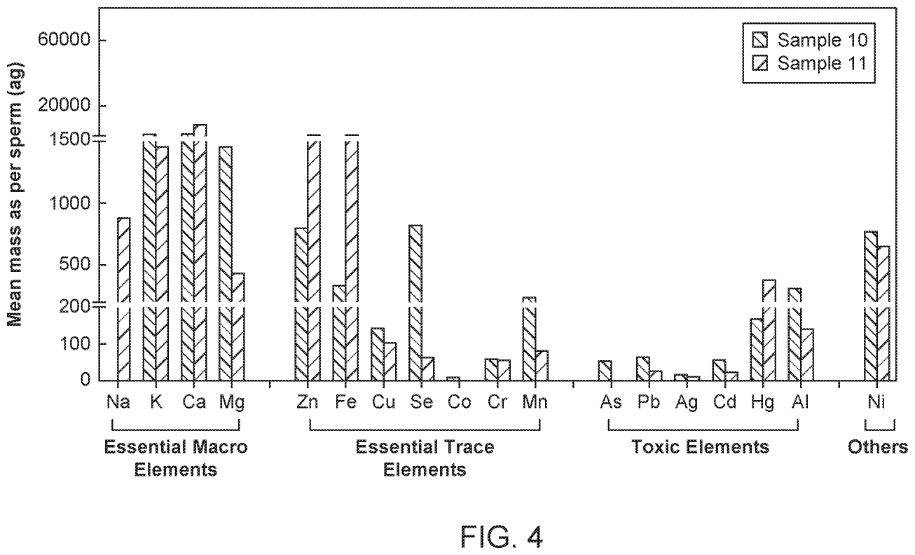

As shown in FIG. 4, an example is provided of a few samples with a relatively high content of other elements in single human sperm. These reference parameters have clinical implications.

As shown in FIG. 5, the sc-ICP-MS single signal's kinetic dynamics characteristics of specific element are provided. The figure is based on the sc-ICP-MS signal dynamics characteristics of the iron and copper elements in single human sperm cells of the same sample as an example, showing the element-specific characteristics unique to the cell, including dwell time, peak time of a single signal spike and its ratio relating to dwell time, dynamic kinetic constant or area of pre-peak of spike, and dynamic constant or area of post-peak of spike.

Different cells contain different element content; while certain elements in different cells show unique ICP-MS signals. Thus, such parameters can distinguish specific properties of different cells. For example, in cultured mouse epididymal epithelial DC2 cells, human fetal kidney 293T cells, human cervical cancer HeLa cells, and human gastric cancer SNU-1 cells, the time and constant of the sc-ICP-MS signal related characteristics of different elements are distinguishable. The characteristics of these ICP-MS signal dynamic kinetics have important biological significance and far-reaching implications in relevant to pathological status evaluation, and thus have clinical application implications.

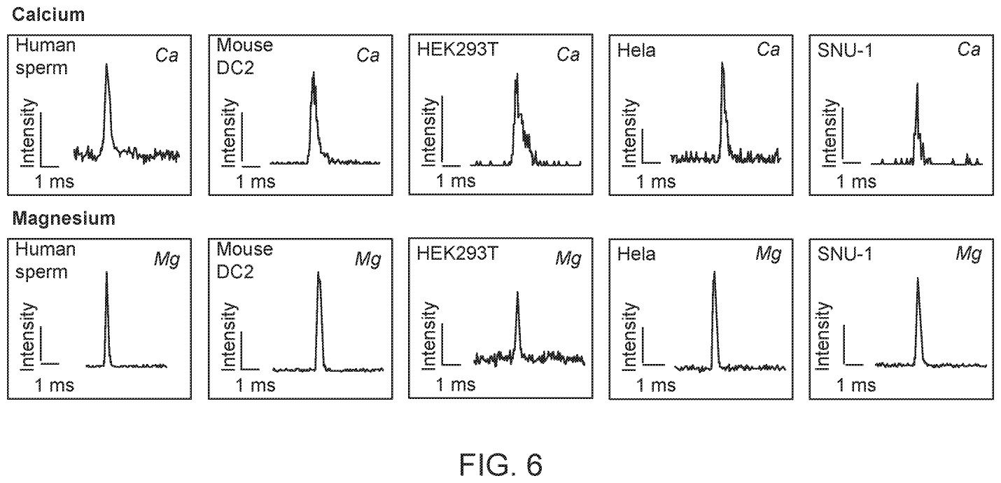

FIG. 6 shows the signal characteristics of essential macro-elements (calcium and magnesium) in different types of cells.

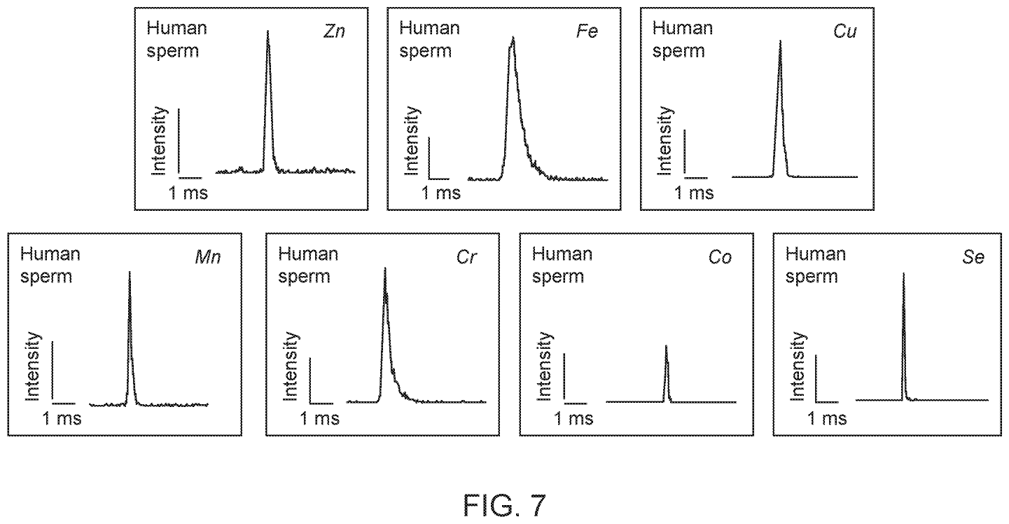

FIG. 7 shows the signal characteristics of essential trace elements in human sperm, including zinc (Zn), iron (Fe), copper (Cu), manganese (Mn), chromium (Cr), cobalt (Co), selenium (Se), etc.

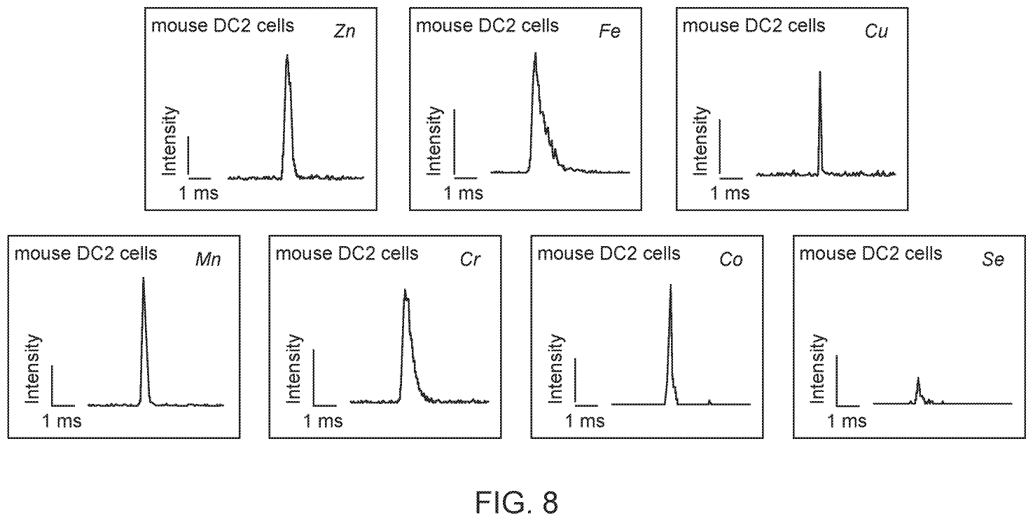

FIG. 8 shows the signal characteristics of the necessary trace elements in mouse epididymal epithelial DC2 cells, including zinc (Zn), iron (Fe), copper (Cu), manganese (Mn), chromium (Cr), cobalt (Co), selenium (Se), etc.

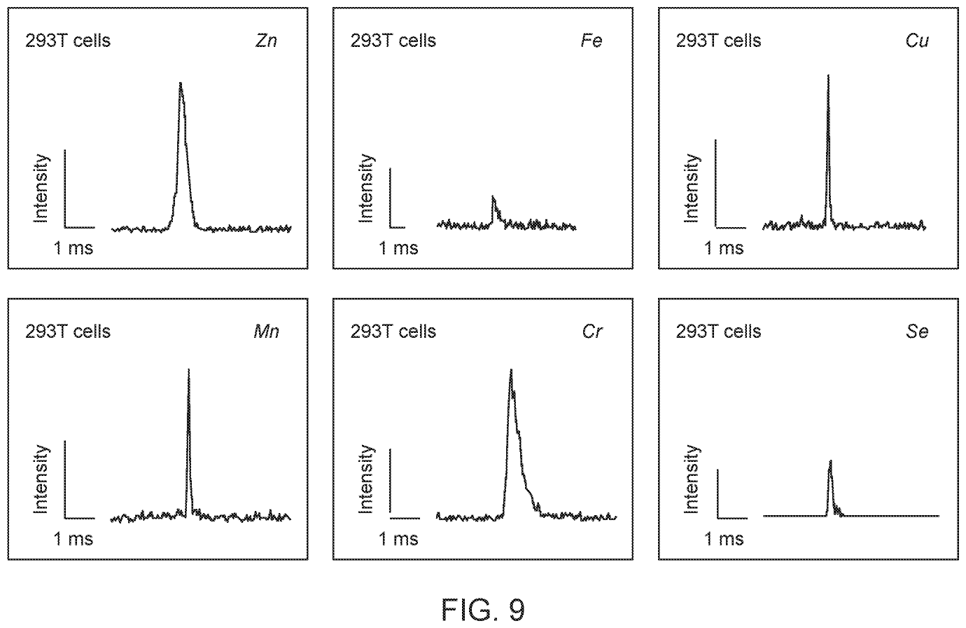

FIG. 9 shows the signal characteristics of essential trace elements in human fetal kidney 293T cells, including zinc (Zn), iron (Fe), copper (Cu), manganese (Mn), chromium (Cr), selenium (Se), etc.

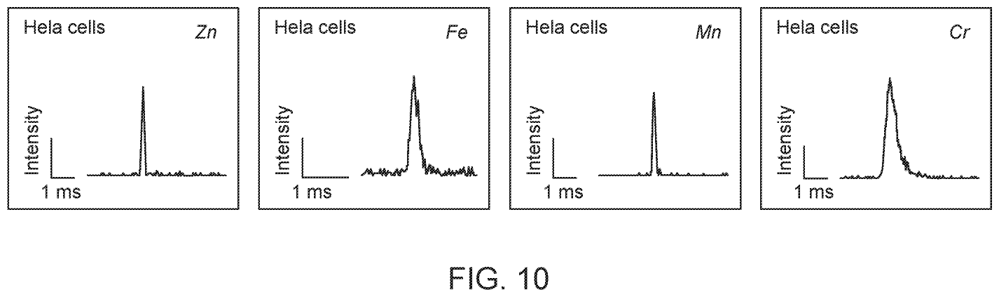

FIG. 10 shows the signal characteristics of essential trace elements in human cervical cancer Hela cells, including zinc (Zn), iron (Fe), manganese (Mn), chromium (Cr), etc.

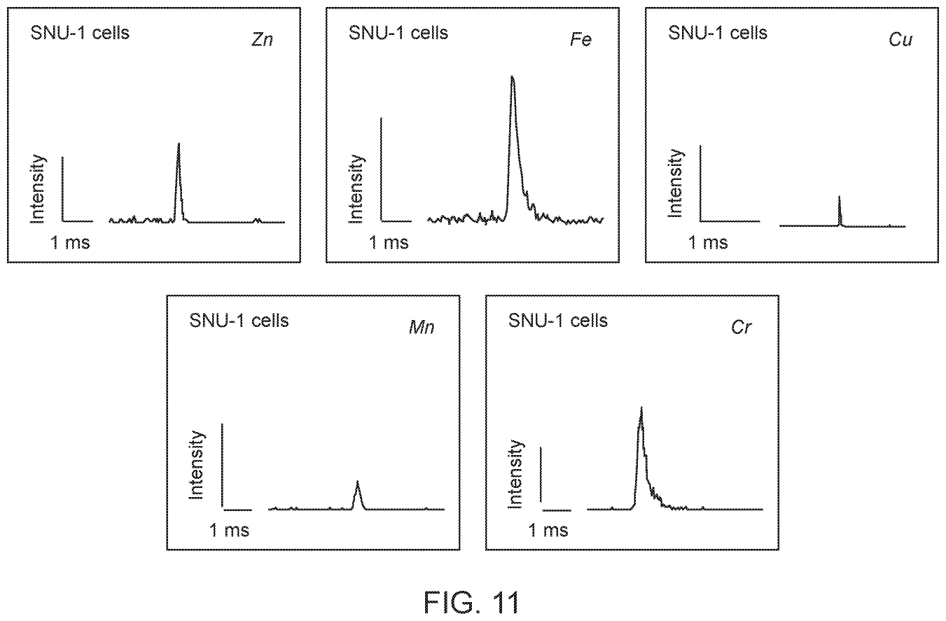

FIG. 11 shows the signal characteristics of essential trace elements in human gastric cancer SNU-1 cells, including zinc (Zn), iron (Fe), copper (Cu), manganese (Mn), chromium (Cr), etc.

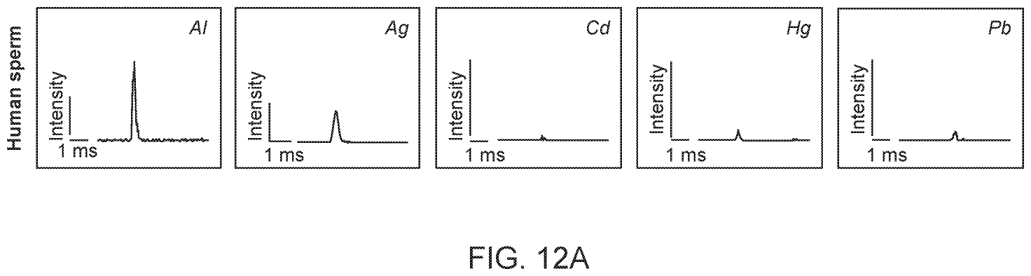

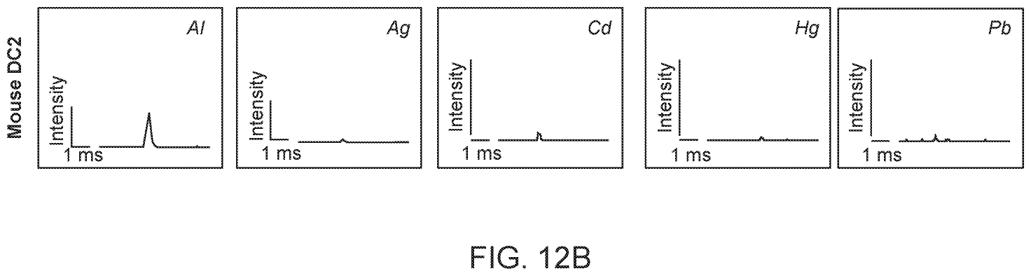

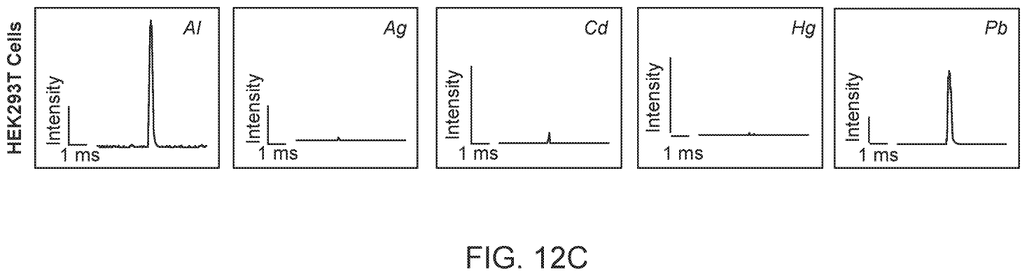

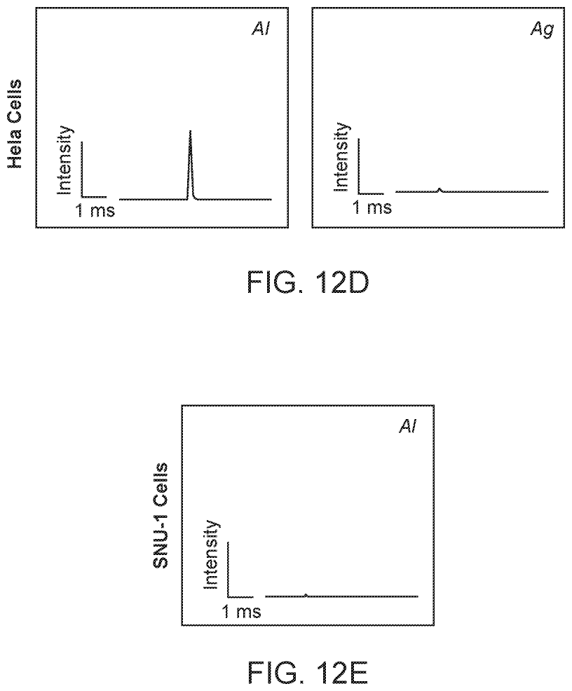

FIG. 12 provides the signal characteristics of toxic elements in different types of cells. FIG. 12A shows human sperm. FIG. 12B shows mouse epididymal epithelial DC2 cells. FIG. 12C shows human fetal kidney 293T cells. FIG. 12D shows human cervical cancer Hela cells. FIG. 12E shows human gastric cancer SNU-1 cells. The signal values and kinetic characteristics of these toxic elements have biotoxicological and pathological significance, and can be used in clinical diagnosis.

### Example 2: Uses of and Methods for Elementomic Characterization Analysis in Diagnosis and Prognosis of Medical Diseases and Conditions

Elementomic characterization analysis of normal and dysfunctioned human spermatozoa using single-cell ICP-MS: Sperm dysfunction is the main cause of male infertility nowadays, but the diagnostic failure rate still accounts for 30% to 70% of clinical cases, most of which are still labelled as idiopathic. This is partly because the traditional assessment methods of sperm function cannot fully meet the clinical needs. In an attempt to determine whether the element bioavailability profile in single human spermatozoa can be an approach to clarify the functional association with male factor infertility, single-cell inductively coupled plasma mass-spectrometry (sc-ICP-MS) was employed. To this end, the elementomic bioavailability profiles were characterized of human normal spermatozoa and dysfunctioned sperm with oligoasthenozoospermia. It was found that not only the elementomic profiles, but the unique signatures of specific elements of human spermatozoa were related to the risk of oligoasthenozoospermia. Without wishing to be bound by the theory, the single-cell elementomic characterization analysis approach can be used to analyze spermatozoal function and other health conditions related to male fertility.

Human sperm sample preparation: All the included human studies were approved by the Medical Ethics Review Board of Shanghai Institute for Biomedical and Pharmaceutical Technologies (original “Shanghai Institute of Planned Parenthood Research”), as well as the Research Ethics Board of ShanghaiTech University. The semen samples were collected using the method introduced by the WHO. With the patients' consent, the remaining sperm samples were collected without hindering clinical uses. Samples of a total 68 specimen were collected from the hospitals including the Reproductive Medicine Center of Zhongshan Hospital affiliated to Fudan University, Shanghai Jiai Genetics and IVF Institute. Within these examined samples, 34 of them were original semen samples and 34 were capacitated sperm samples for in vitro fertilization (IVF) or intracytoplasmic sperm injection (ICSI) assays. Nine of them had both original semen samples and had been capacitated. All these samples were collected by masturbation after 3-5 days of recommended abstinence from sexual intercourse or masturbation. Sperm count and motility of the harvested sperm fluids were measured (37° C.) by the computer-aided sperm analysis (CASA) and the sperm samples for IVF/ICSI were treated by density gradients according to WHO laboratory manual for the examination and processing of human semen at the site of sample collection. The related information is shown in Table 2.

Samples were prepared to a sperm density lower than 1×10⁶ per ml from four physiological status of sperm cells: 1) normal semen without undergoing capacitation (normal sperm, without capacitation); 2) normal viable motile sperm treated with a standard procedure of density gradient centrifugation in a commercial HTF-HEPES buffer for sperm to undergo capacitation during centrifugation (normal viable DGC capacitated sperm); 3) asthenozoospermia or oligozoospermia sperm without capacitation (oligoastheno); 4) the viable DGC capacitated sperm but categorized as oligoasthenozoospermia (capacitated oligoastheno). The oligoasthenozoospermia was categorized according to the lower reference limits for semen characteristics as stated in the 6^{th} edition of WHO laboratory manual for the examination and processing of human semen.

Normal samples were those semen with concentrations greater than 15×10⁶ per ml, and asthenozoospermia samples were identified with concentration less than 15×10⁶ per ml in original semen, whereas oligozoospermia were the samples with sperm motility (PR+NP %) less than 40% (Agarwal et al., 2021; and WHO, 2010, WHO laboratory manual for the examination and processing of human semen Fifth edition.).

TABLE 2
Analysis of Semen Parameters of Human Subjects Included
in This Study (Mean ± Standard Error of Mean).

| | | | For IVF/ICSI (capacitated) | |
|---|---|---|---|---|
| | Original Semen | | Normal | |
| Subject | Normal | Oligozoospermia/ | capacitated | Oligozoospermia/ |
| parameters | spermatozoa | Asthenozoospermia | spermatozoa | Asthenozoospermia |
| Sperm count | 214.3 ± 30.0 | 58.8 ± 30.1 | 148.7 ± 19.7 | 73.6 ± 24.4 |
| (×10⁶) | (n = 29) | (n = 2) | (n = 28) | (n = 6) |
| Sperm | 51.5 ± 5.0 | 20.8 ± 5.7 | 70.7 ± 4.9 | 39.2 ± 7.0 |
| concentration | (n = 29) | (n = 5) | (n = 28) | (n = 6) |
| (×10⁶ sperms/ml) | | | | |
| Total | 56.3 ± 2.4 | 28.8 ± 7.1 | 57.7 ± 2.0 | 25.8 ± 3.0 |
| motility | (n = 29) | (n = 5) | (n = 28) | (n = 6) |
| (%) | | | | |
| Progressive | 31.7 ± 2.8 | 12.5 ± 10.6 | 30.1 ± 1.5 | 14.2 ± 3.0 |
| motility | (n = 12) | (n = 2) | (n = 28) | (n = 6) |
| (%) | | | | |
| Age | 34.6 ± 1.1 | 32.0 ± 2.6 | 39.8 ± 1.4 | 38.5 ± 3.2 |
| (years) | (n = 29) | (n = 5) | (n = 28) | (n = 6) |

Aliquots of the harvested sperm semen in 2-ml Eppendorf tubes were then sent in a styrofoam protected container at room temperature (RT) within two hours to the sc-ICP-MS laboratory for the subsequent experimental preparations. The spermatozoa were spin down at 500 g for 10 min and fixed in 4% PFA for 15 min at RT, followed with three washes with pure water before dilutions in milli-Q water to desired concentrations for sc-ICP-MS measurements. All sperm samples were collected during fall or winter. The spermatozoa were immediately aspirated with a 1-ml injection infusion pumping (Harvard Apparatus) into the aspiration chamber for sc-ICP-MS analysis. For the measurement of total cells, the cells were digested with concentrated nitric acid at 99° C. for 30 min. Then, the digested solution was diluted with milli-Q water into 2% HNO₃ and determined by ICP-MS.

Cultured cell sample preparation: DC2 cell line (one of mouse epididymal epithelial cell lines, and a gift from Dr. Yong-Lian Zhang's lab) was cultured in an incubator at 33° C., 5% CO₂ with Full-IMDM (Iscove modified Dulbecco medium (IMDM) supplemented with 1 nM 5α-dihydrotestosterone and containing 10% (v/v) fetal bovine serum (FBS), penicillin (100 g/mL) and streptomycin (100 g/mL)). Human gastric cancer cell line SNU-1 (a gift from Dr. Shuo Shi at ShanghaiTech University) was cultured in an incubator at 37° C., 5% CO₂ with Roswell Park Memorial Institute (RPMI) 1640 culture medium supplemented with streptomycin (100 g/mL), penicillin (100 U/mL), glutamine (2 mM), and 10% (v/v) FBS (complete medium). HEK293T and Hela cell lines were cultured in an incubator at 37° C., 5% CO₂ with full DMEM (Dulbecco's Modified Eagle Medium), containing 10% (v/v) FBS, penicillin (100 g/mL) and streptomycin (100 g/mL). The cultured cells were digested into a single dispersed cell with TrypLE, washed three times with PBS, spin down at 1000 g for 5 min and either fixed in 4% PFA for 15 min at RT or unfixed, followed with three washes with pure water and removal of the cell clumps with a 70-mesh sieve to ensure single cell samples before dilutions in milli-Q water to desired concentrations for sc-ICP-MS measurements.

Sample preparation: The fresh human semen was liquefied and centrifuged according to the conventional semen preparation method. Then the seminal plasma and sperm were separated and the sperm was resuspended and fixed with 4% PFA for 15 minutes. Then the fixative solution was removed by washing, and the sperm samples were used directly or stored at four degree for later use.

The sample was diluted with a diluent to a detect concentration of below 3×10⁶/ml.

The samples were then loaded to an sc-ICP-MS instrument for detection: a specific single-cell nebulizer and spray chamber were installed. A tuning solution (2% v/v nitric acid containing 10 μg/L Li, Be, Mg, Fe, In, Ce, Pb and U) was used to determine appropriate instrument detection parameters. Gold particle standards (such as 50 nm gold particles) was used to determine the single cell transmission efficiency which was generally in the range of 40%-60%. Standard solutions were prepared of different concentrations of different elements (such as Na, K, Ca, Mg, Zn, Fe, Cu, Se, Co, Cr, Cd, Mn, As, Hg, Pb, Ag, Al, Ni, etc.). Standard curves were plotted accordingly. The concentrations of the standard solutions of Hg were 0.5 part per billion (ppb), 1 ppb, and 2 ppb. The concentrations of the standard solution of Ca were 50 ppb, 100 ppb, and 200 ppb. The concentrations of the standard solution of other elements were 5 ppb, 10 ppb, and 20 ppb. All standard solutions were prepared in ultrapure water. In order to eliminate polyatomic interference and to obtain a high signal-to-noise ratio, measurements were performed in the dynamic reaction cell (DRC) mode, using ammonia as the reaction gas for the detection of K, Ca, Cr, and Fe in single cells, or using oxygen as the reaction gas for the detection of As and Se in single cells by detecting the oxidation reaction products of AsO and SeO as the analysis species. Other elements were measured in standard mode. The sample spike signals were sampled for 50 s with a dwell time of 50 μs (i.e., digitizing at a frequency of 50 MHz), and the total sample volume consumed by detecting 18 elements was about 400 μL. The normal working conditions of sc-ICP-MS are shown in Table 3 below. Exemplified results can be found in FIGS. 1-5.

TABLE 3

| Instrumental | Single Cell ICP-MS | |
|---|---|---|
| Parameters | Analysis | |
| ICP parameters | | |
| Nebulizer | Meinhard HEN Cyto Nebulizer | |
| Spray chamber | Asperon ™ Spray Chamber | |
| Torch | Glass torch | |
| ICP RF power | 400 W-1600 W | |
| Plasma gas flow | 15 | L/min |
| Auxiliary gas flow | 1.2 | L/min |
| Nebulizer gas flow | 0.38 | mL/min |
| Sample Flow Rate | 0.016-0.025 | mL/min |
| Torch | Fixed 2.0 mm Injector | |
| | Quartz Torch | |
| Cones | Nickel, Tri-cone Design | |
| Measurement | | |
| parameters | | |
| Data acquisition | Standard | |
| mode | | |
| TRA duration | | |
| time (s) | | |
| Dwell Time | 50 | μs |
| Scan Time | 50 | s |
| Transport Efficiency | 41-60% | |
| DRC parameters | | |

| | |
|---|---|
| RPa | 0 |
| RPq | 0.45 ml/min for ⁵⁶Fe⁺ and ⁴⁰Ca⁺, |
| | 0.25 ml/min for others |
| DRC gas (NH₃) | 0.45 ml/min for ⁴⁰Ca⁺ ⁵²Cr⁺ and ⁵⁶Fe⁺, |
| | 1 ml/min for ³⁹K⁺, |
| DRC gas (O₂) | 0.45 ml/min for ⁹⁶Mo(SeO)⁺ |
| | and ⁹¹Zr(AsO)⁺ |

Note:
DRC: dynamic reaction cell.

As illustrated in FIGS. 6-12, examples show characteristics of sc-ICP-MS signal dynamics of different elements in a single human sperm, including the dwell time of a single signal (whole-peak dwell time), the peak time of a single signal during the dwell time, the pre-peak dwell time and raising tau constant before the peak, and the post-peak dwell time and tailing tau constant after the peak. It was found that different cells not only contain different elements (bioavailability), but specific elements also have unique ICP-MS signals of special elements in single cells, and they can be identified in different types of cells. For example, the time and constant of the sc-ICP-MS signal related characteristics of different elements can distinguish different cell types, such as cultured mouse epididymal epithelial DC2 cells, human fetal kidney 293T cells, human cervical cancer HeLa cells, human gastric cancer SNU-1 cells. The biological significance, relevant pathological significance, and clinical application prospects of these signal dynamics characteristics are under investigation.

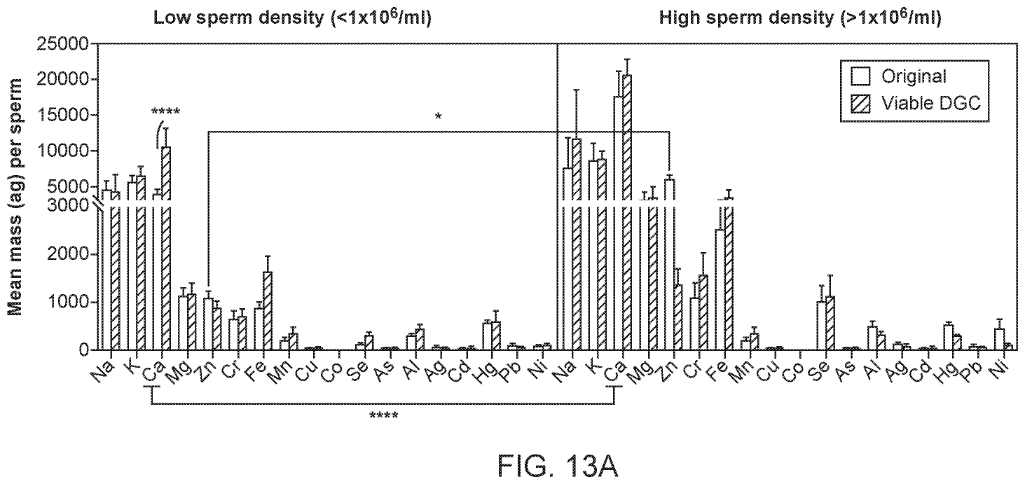

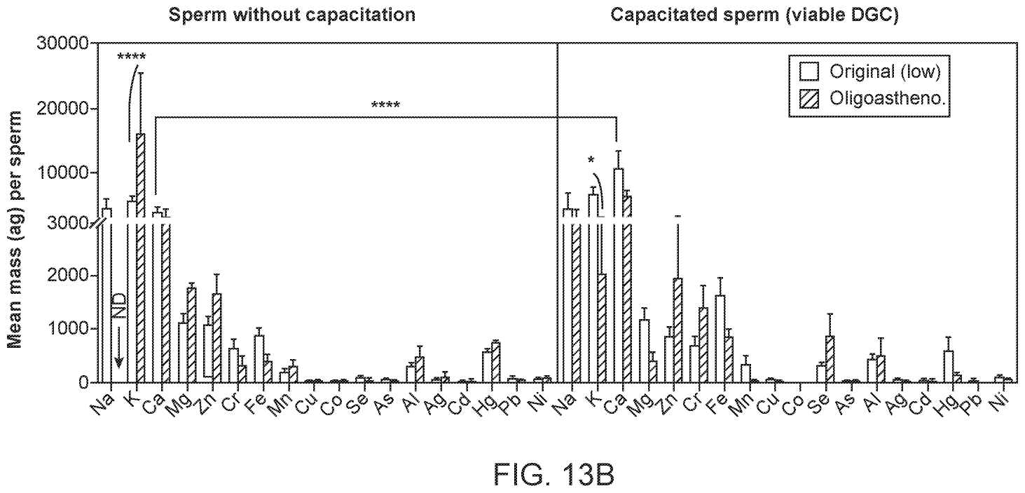

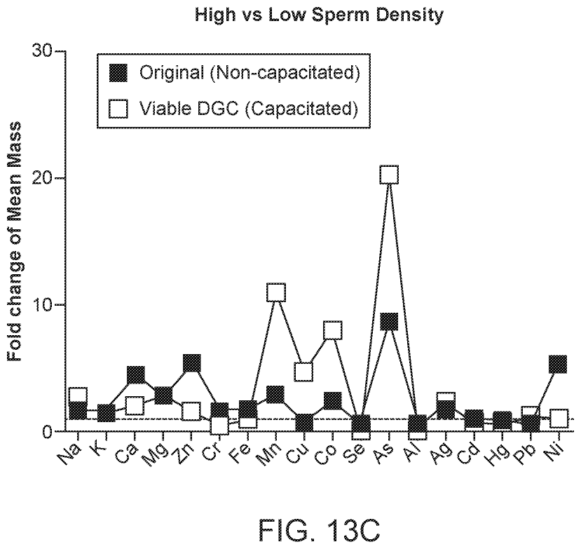

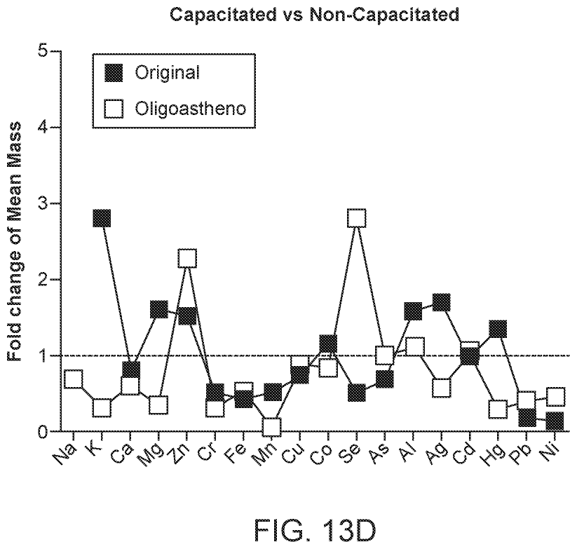

FIGS. 13-14 provide characteristics of the mean mass profile of elements in single human sperm of normal and oligoasthenospermia samples.

When analyzing the elemental mean mass data obtained from the ICP-MS readings, it was noticed that the values of some elements changed abruptly from reading to reading. In order to determine the cause of this change, relationship between element contents versus the dilution factor and the sperm density of samples were analyzed. The results showed that at a lower cell density, the mean masses of elements were relatively stable. When the cell density was higher than around 1×10⁶ per ml, the averaged contents of Na, Ca, Mg and Zn elements became significantly correlated and increased with the increase of cell. In some embodiments, the results also showed that the averaged contents of most elements were stable when the sperm sample was diluted 10 times or more, regardless of original cell concentration. Although no statistically difference was determined with the tested dilution factor of samples, a tendency of higher mean mass was determined with original dilution factor for the Na, K, Ca, Mg, Zn, and Fe elements. For these elements, further analyzes were then performed with dilution factor at 10 times or higher. Therefore, for the statistical analysis, the mean mass of elements was divided into two groups, with a lower or higher cell density than 1×10⁶ per ml, both with a dilution at 10 times or more.

The results showed that only the mean content of Ca was significant increased when comparing the normal spermatozoa diluted from original semen samples and the viable motile sperm from the samples treated with a procedure of density gradient centrifugation (DGC) in a HTF buffer, which also allowed sperm to undergo capacitation and to be harvested with good motility (FIG. 13A). This result is consistent with the fact that Ca is essential for sperm capacitation and hyperactivation. During these processes, a large amount of calcium ions influxes into the sperm cells, thus activates the sperm cells and prepares them for acrosome reaction and fertilization (Ickowicz et al., 2012, Asian J Androl 14, 816-821; and Navarrete et al., J Cell Physiol, 2015, 230, 1758-1769). Disturbance of calcium homeostasis in sperm leads to defects in sperm motility and capacitation, which in turn leads to male infertility in mice, with accompanied by an increase in the resting level of intracellular calcium in sperm cells (Okunade et al., 2004, J Biol Chem 279, 33742-33750; and Schuh et al., 2004, J Biol Chem 279, 28220-28226).

The results also showed that in sperm with a higher cell density, the mean mass of Zn element was significantly increased under control condition but not after DGC-capacitation procedure (FIG. 13A). Some other elements also showed greater mean mass content in sperm with higher density, although there was no statistical difference. Without wishing to be bound by the theory, two mechanisms were proposed herein to explain this phenomenon. The first mechanism is that this was due to the matrix background carried over from the original semen plasma. Supporting this mechanism, the fold change of mean mass of most elements, including macro-essential elements Na, Ca and Mg, and trace-essential elements Zn, Cr, Fe, Cu and Se, were increased at higher cell density (FIG. 13C). In addition, it has been reported that the cellular surface glycocalyx regulates the ambient sodium homeostasis and thus controls permeability and sodium-potassium pump activity in pathophysiological conditions (Korte et al., 2012, Pflugers Arch 463, 269-278). Moreover, it was found that the mean mass of K was substantially inversely correlation the Na of spermatozoa (FIG. 14A). Without wishing to be bound by the theory, proposed herein is another mechanism that this might be due to the ions trap in the carbohydrate-rich glycocalyx coating on the sperm membrane surface, which is known to be about 20-60 nm thick (Fabrega et al., 2012, Reprod Fertil Dev 24, 619-630; Schroter et al., 1999, Hum Reprod Update 5, 302-313; and Tecle and Gagneux, 2015, Mol Reprod Dev 82, 635-650). Further experiments are under investigation to confirm this mechanism and the physiological significance behind it.

It was found that, compared to control sperm diluted from original semen condition, the average Ca mass of sperm treated with the DGC capacitation procedure also increased significantly, but no changes in Ca mass content was detected in the sperm with abnormal oligoasthenospermia (FIG. 13B). In addition, compared with normal sperm under control conditions, the K mean mass of oligoasthenospermia sperm increased significantly, but the K average mass of DGC-capacitated oligoasthenospermia samples reduced. Compared with the normal control, the fold change of mean mass of essential elements K, Mg and Zn increased after DGC capacitation procedure, while only Zn increased in oligoasthenospermia sperm (FIG. 13D).

To verify the accuracy of this sc-ICP-MS method, the single-cell mean mass results of samples with a sperm density lower than 1×10⁶ per ml were compared with bulk analysis by applying a conventional acidic cell digestion ICP-MS method. The cellular elemental contents determined by this sc-ICP-MS analysis and by the commonly used acidic digestion of batch of cells were in a comparable range (FIGS. 14B-14C). Consistently, when comparing the normal spermatozoa diluted from original semen samples, the mean content of Ca increased significantly in viable motile sperm of samples capacitated in DGC condition.

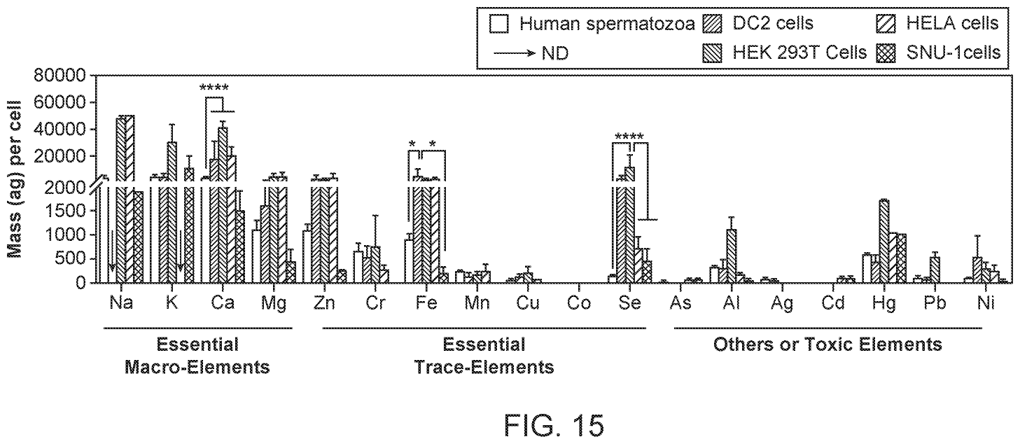

FIG. 15 provides characteristics of elementomic mean mass profiles in normal single human spermatozoon in comparison with somatic cells.

In order to explore whether the mean mass elementomic profile was specific in normal single human spermatozoon, the elementomic profiles were determined in cultured somatic cells, including WT mouse epididymal epithelial DC2 cells, embryonic human fetal kidney 293T cells, human cervical cancer HeLa cells and human gastric cancer SNU-1 cells (FIG. 15). The mean mass results of the different cell types showed that compared with somatic cells, the overall content of essential elements in human sperm was generally less, especially compared with DC2 cells, HELA cells and HEK293T cells: the contents of Ca and Fe were statistically less, while Se was substantially higher in mouse WT DC2 epithelial cells and embryonic human HEK293T cells than in human normal sperm cells. Interestingly, a significantly less amount of Fe or Se content was noticed in cancerous HeLa and/or SNU-1 cells, comparing to WT DC2 cells and embryonic HEK293T cells.

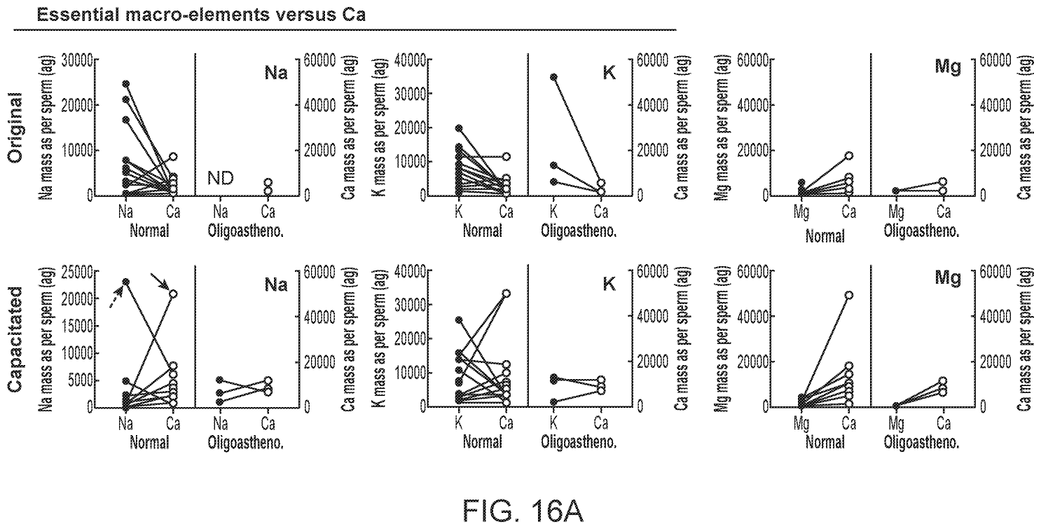

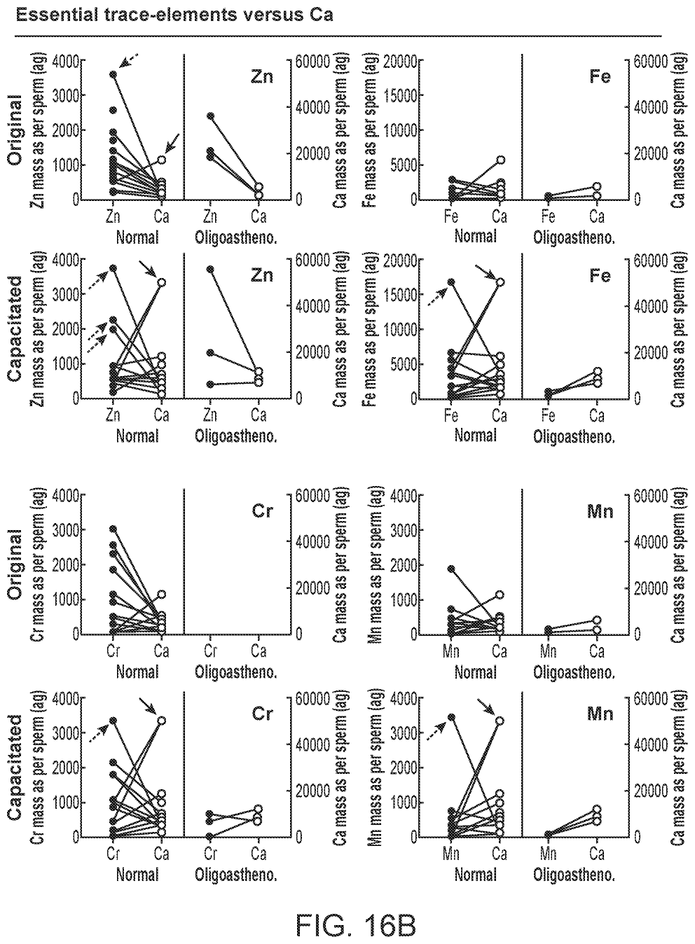

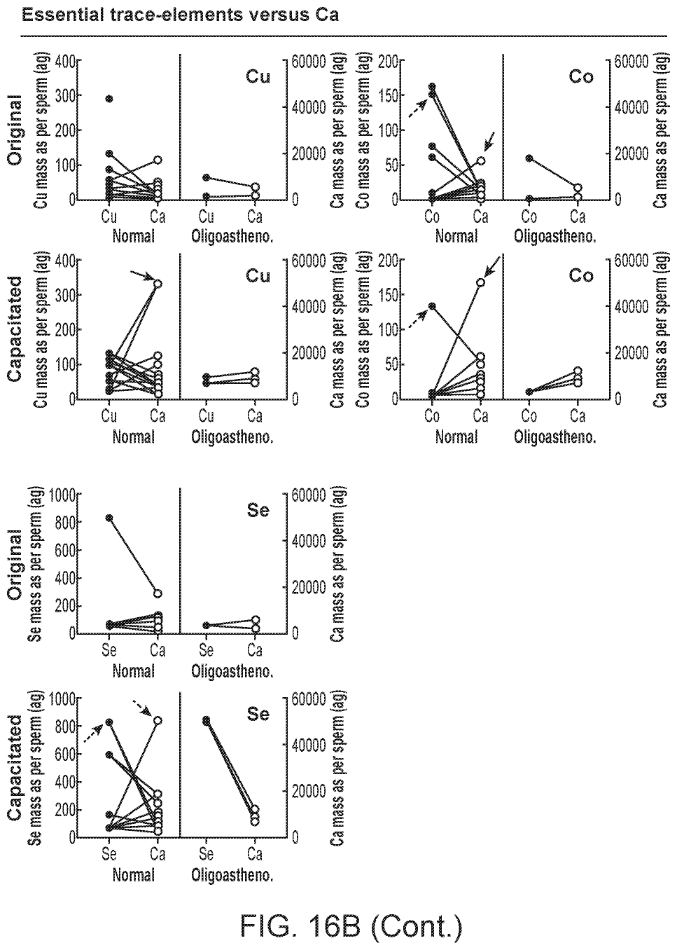

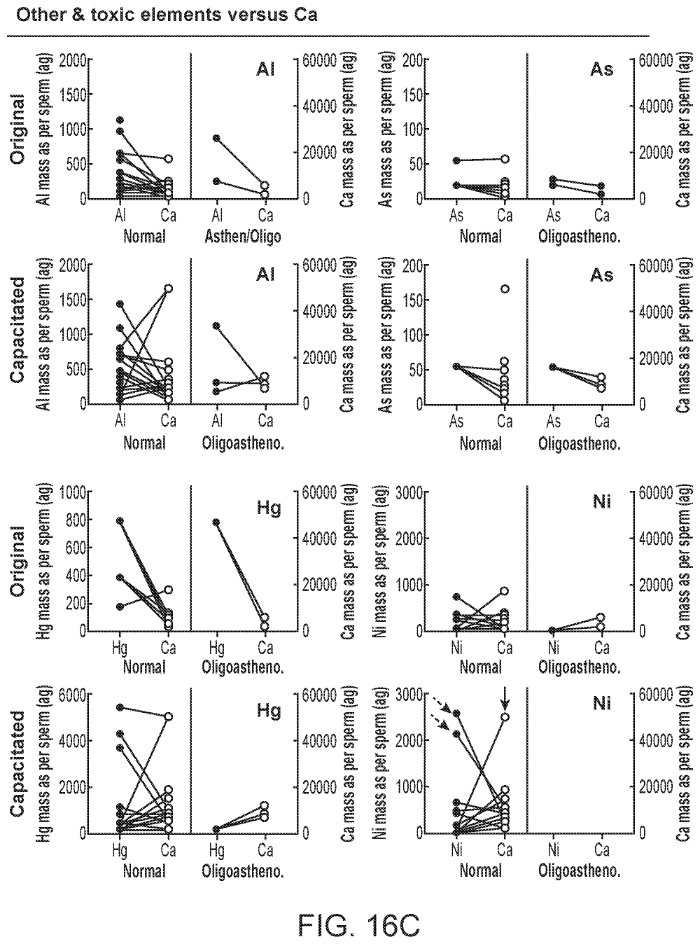

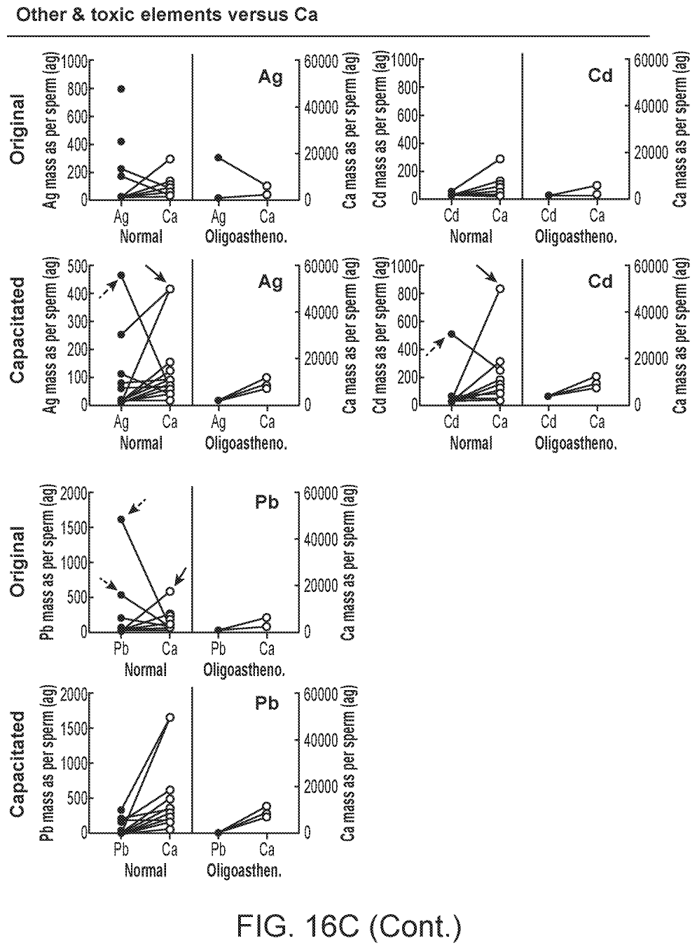

FIGS. 16-21 provide element-to-Ca relation in single cells of the same biological samples.

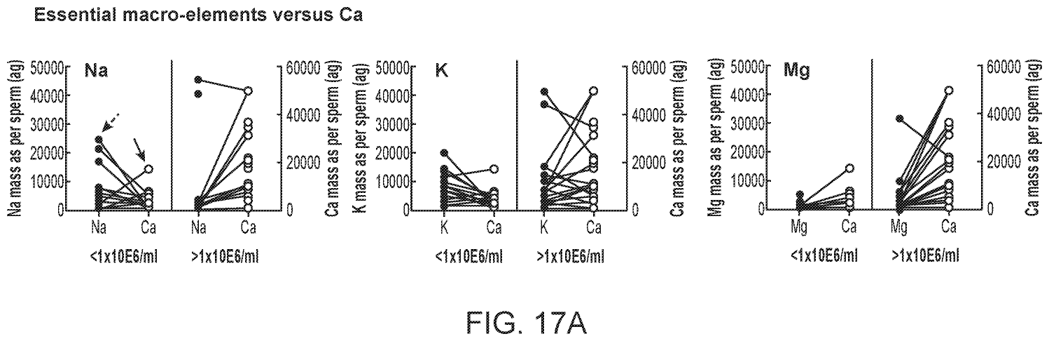

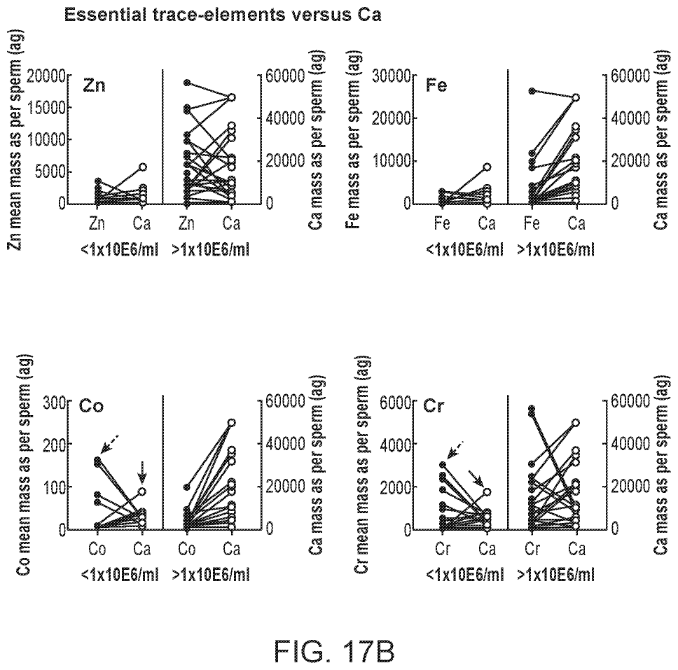

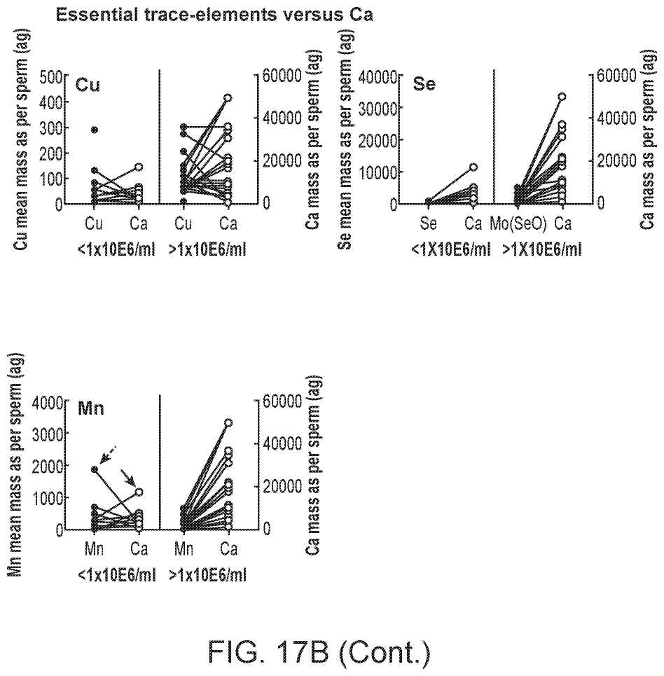

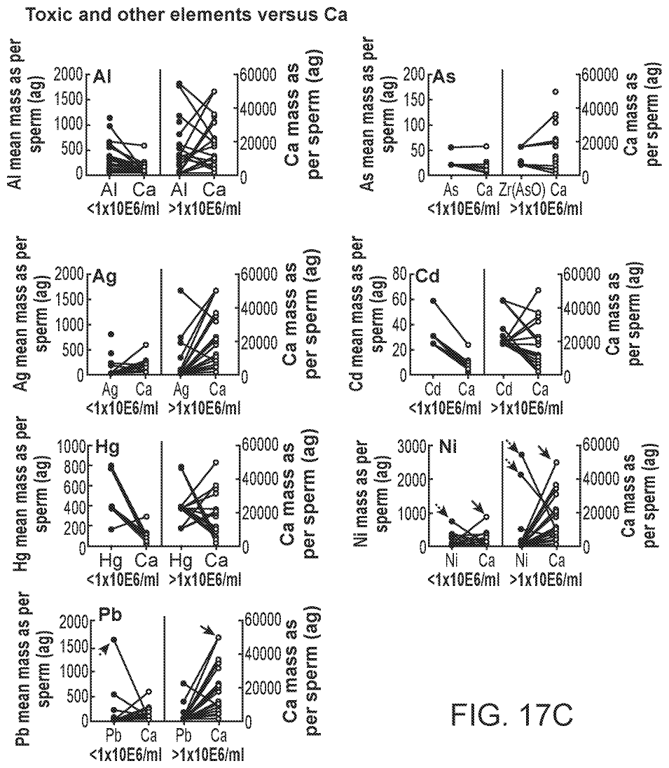

Ca homeostasis is essential for sperm fertilization function and male fertility. Studies suggests that calcium interacts with Pb and that low Pb exposure results in low sperm motility and asthenozoospermia risk (Zhang et al., 2021, Cell Biosci 11, 150). Metal ion homeostasis, particularly transition metals Fe and Cu, plays an indispensable role in male reproduction, though their role is a double-sided coin (Mirnamniha et al.; Nelson; and Tvrda et al.). The interactions of the elements and the underlying physiological relevance are still largely unknown. As a first attempt to understand the inter-relation profile amongst elements, correlation analysis was performed on relation of contents of the determined elements compared to Ca in single cells of spermatozoa and somatic cells. It was found that in the correlation plots of elements versus Ca in human spermatozoa, mean contents of essential elements, including Na, Zn, Co, Cr, Fe, Cu, Mn and Se correlated with that of Ca (FIGS. 16A-16B and FIGS. 17A-17B). No correlation was observed for the essential K and Mg macro metals. In addition, an inverse correlation was also observed for the contents of toxic elements Ag, Cd, Ni and Pb with that of Ca (FIG. 16C and FIG. 17C). The correlation of mean mass of elements versus Ca in culturing somatic cells were also plotted (FIGS. 18-21). The results evidence that Ca homeostasis is important for Na-associated nutrients homeostasis and redox-sensitive element-associated physiological processes as well as toxic elemental detoxification. Further experiments are under investigation to confirm physiological significance of the correlation between elements and specific elementomics at the single-cell level, and to explore the underlying molecular mechanisms of elementomic-network associated physiological processes in normal and abnormal spermatozoa, as well as in somatic cells.

FIGS. 22-25 provide association of elementomic characteristics with oligoasthenozoospermia risk.

The data obtained by sc-ICP-MS provided the distribution patterns of mean mass of elements in individual cells, which reflected the differences of cells in the same population. Based on the average mean mass of elements of a whole population of single spermatozoon, no association between the oligoasthenozoospermia risk and the mean mass contents of elemental contents of spermatozoa was observed. Further evaluated were the mean mass frequency distribution patterns of specific elements of individual spermatozoon prepared from semen samples of normal or oligoasthenozoospermia, treated with or without a capacitation procedure in a HTF solution accompanying by density gradient centrifugation. The results showed that almost all distribution patterns of determined elements of individual spermatozoon did not follow a perfect Gaussian distribution, including the essential macro- and micro-elements like Na, K, Ca, Mg, Zn, Fe, Cr, Cu and Mn. In some essential micro-elements like Co and Se and the toxic or other elements like Al, Cd and Ni, the frequency distributions can be described with a Poisson distribution, assuming a perfectly random arrival of ions to the detector, as described previously for ICP-MS spike events detection (Cornelis and Hassellov, 2014, J Anal Atom Spectrom 29, 134-144; and Wang et al., 2015). Statistical analyses showed that comparted with control group, the distribution patterns of Na, Ca, Mg, Zn and Al in samples prepared with normal semen processed by the capacitation procedure were significantly different. In addition, distribution patterns for K, Fe, Cr, Cu and Se in spermatozoa with abnormal oligoasthenozoospermia were significantly different from that of control group.

Taking the distribution patterns of Fe mean mass contents as an example, the resulted showed that in the entire defined spermatozoa population of a single preparation of normal semen, regardless of the challenge of capacitation procedure, was composed of various subpopulations, of which there were at least a low Fe-contented population and another high Fe-contented population (FIG. 23A). In both high or low-contented populations of signals of the same biological samples, subpopulations were also identified preferentially in normal than abnormal spermatozoa with oligoasthenozoospermia (FIG. 23B). In the abnormal oligoasthenozoospermia sample preparations, no high-Fe content subpopulation was observed, regardless with or without capacitation challenges (FIG. 23A). Statistical analyses showed that Fe distribution pattern was significantly altered after capacitation-induction procedure compared to control group of normal spermatozoa (FIG. 23A). In abnormal spermatozoa, the distribution was statistically significant compared to normal semen control group, although no difference was found between capacitated abnormal spermatozoa and the original abnormal spermatozoa without capacitation. Interestingly, the distribution pattern of abnormal sperm after capacitation was more random than without capacitation. These results evidence that element distribution pattern revealed element distribution patterns is a factor related to oligoasthenozoosperma risk, especially the Fe content in single spermatozoon, consistent with the notion of metal homeostasis in regulation of sperm function and male fertility (Mirnamniha et al.; Nelson; and Tvrda et al.).

Unique elemental dynamic kinetics of ICP-MS spike signals were identified in normal, oligoasthenozoospermia spermatozoa and somatic cancerous cells.

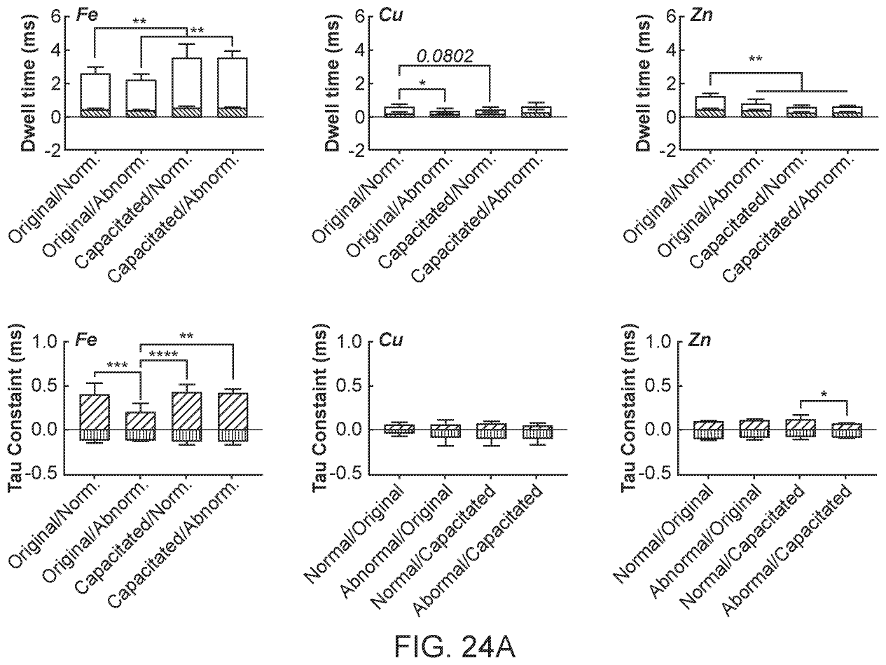

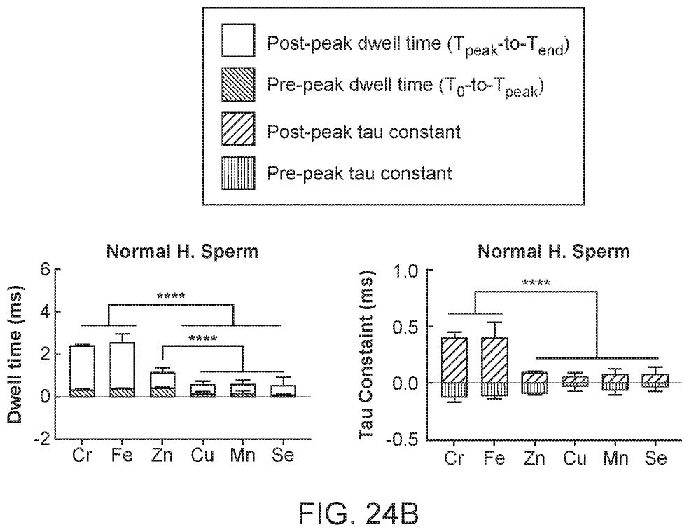

In order to determine whether there is a difference in the kinetic dynamics of specific types of elements between normal and oligoasthenozoospermia spermatozoa (FIG. 24A), further evaluated were the dynamic kinetic parameters ICP-MS spike signal characteristics, taking Fe and Cu of normal spermatozoa as examples as demonstrated in FIG. 5. These parameters included spike signal dwell time, which consists of pre-peak time and post-peak time to reach or curtail from, respectively, the peak of a single signal within the dwell time, and the parameters of dynamic kinetic constants before and after the spike peak within the dwell time, as well as the areas under the defined peaks. In an attempt to define the characteristics, the exported ICP-MS spike signal tracings were analyzed and the parameters were determined manually. For the kinetics before and after the spike peak as per single signals, the time constants were obtained by fitting the raising or curtailing of pre-peak or post-peak dynamics with a standard alpha exponential function.

Taking Fe, Zn and Cu as examples, evaluated were the sc-ICP-MS element signal peak style patterns, where Fe represents an example of elements with asymmetric trailing peak style, and Cu represents an example of elements with peri-symmetrical peak style (FIG. 24A). The results showed that whether it was normal or abnormal oligoasthenozoospermia sperm, after being challenged by the capacitation procedure, the post-peak dwell time of Fe of spermatozoa was significantly increased. No difference of dwell time was observed between normal and abnormal spermatozoa. Compared with normal control sperm and capacitation-stimulated sperm, the post-peak tau constant of abnormal sperm was substantially reduced. Regarding Cu, the dwell time in abnormal spermatozoa was reduced compared to normal group under uncapacitated condition, whereas no differences in dwell time or tau constant between the two groups after capacitation stimulation. Regarding Zn, compared to normal group or uncapacitated sperm, the whole-spike dwell time of normal sperm was significantly higher than the abnormal group, either before or after capacitation stimulation. As for the tau constant of Zn, it was significantly decreased after capacitation in the abnormal group compared to the normal spermatozoa.

The kinetic dynamics of specific types of elements in normal human spermatozoa, including Cr, Fe, Zn, Cu, Mn and Se were also summarized for comparison (FIG. 24B). The dwell times for the Cr and Fe were significantly higher than other elements such as Zn, Cu, Mn and Se, whereas Zn was also significantly higher than Cu, Mn and Se. Regarding the tailing tau constants of Cr and Fe were significantly higher than other elements. The physiological implication of this special kinetic profile in spermatozoa is still under investigation.

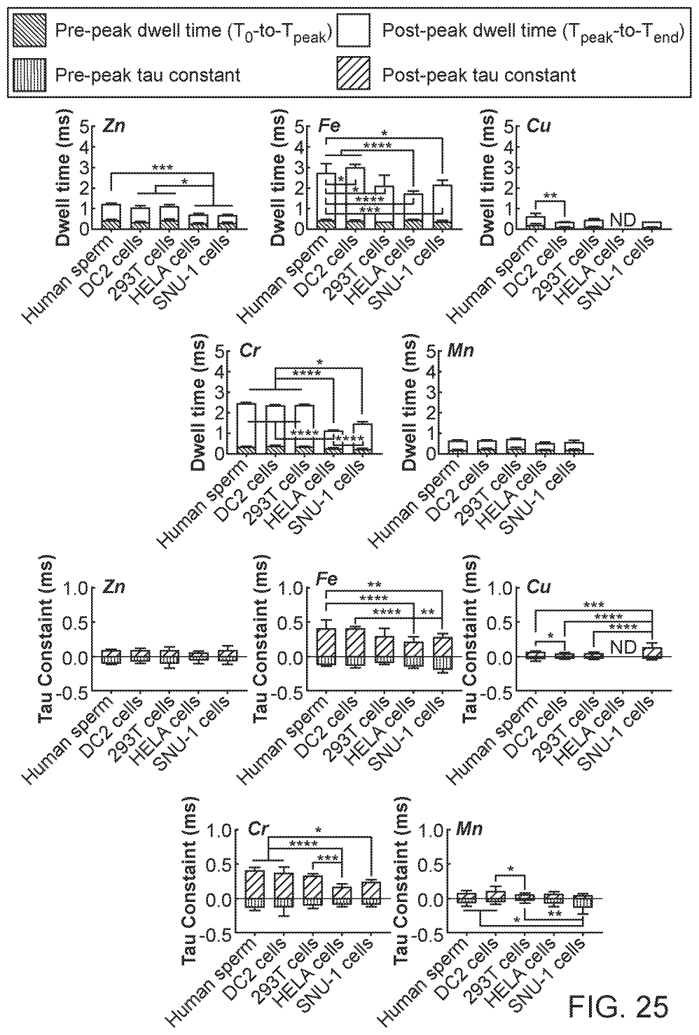

To determine whether the single elemental ICP-MS signal characteristics were specific to spermatozoa, further evaluated were the dwell time (full-peak, pre-peak and post-peak dwell time) and dynamic peak-related kinetic constants of essential micro elements in other cultured somatic cells (FIG. 25). In general, the dwell time for Fe and Cr elements was substantially longer than Zn, Cu and Mn elements, regardless of cell types. Compared with normal human spermatozoa, the full-peak and post-peak dwell time of Zn, Fe, Cu, and Cr was significantly different from those of various somatic cells, while the pre-peak dwell time was not. No difference was observed in the dwell time of Mn between human spermatozoa and various somatic cells. Regarding time constants, the post-peak kinetic tau constants for Fe, Cu and Cr elements in human spermatozoa were prominently different from other somatic cells. For the pre-peak dwell time, only Mn element was significantly different in SNU-1 cells compared with human spermatozoa and 293T cells, while no difference was observed in Zn, Fe, Cu and Cr elements in tested cell types.

Overall, these results showed for the first time that the different dynamic ICP-MS peak patterns and kinetics of different distribution of different elements in sperm cells and some somatic cells, including cancerous cells, were distinguished and potentially related to pathological status. To support this hypothesis, the results showed that the single ICP-MS spike signal characteristics of elementomic in single spermatozoon were correlated with the risk of oligoasthenozoospermia. Without wishing to be bound by the theory, different peak patterns of different elements of the same cell population represent a unique elementomic signature of a given particular cell type, which may be related their status under defined physiological or pathophysiological conditions. The signal values and kinetic characteristics of these elements have biotoxicological and pathological significance, and have the potential to be used in clinical diagnosis.

The same parameters of other examined elements of all cell types examined in this study are also determined using the same analytical approach. These parameters include all characteristics of specific elemental signals of a given cell type with unique elemental and morphological features. Experiments are under investigation to confirm the clinical significance and other potential application of these characteristic parameters, such as for the diagnosis or prognosis of male infertility or cancer or other disease.

Additional experiments are under investigation to confirm the physiological and pathological significance of the sub-populations of elements in single human sperm cells under physiological or pathological conditions for clinical diagnosis.

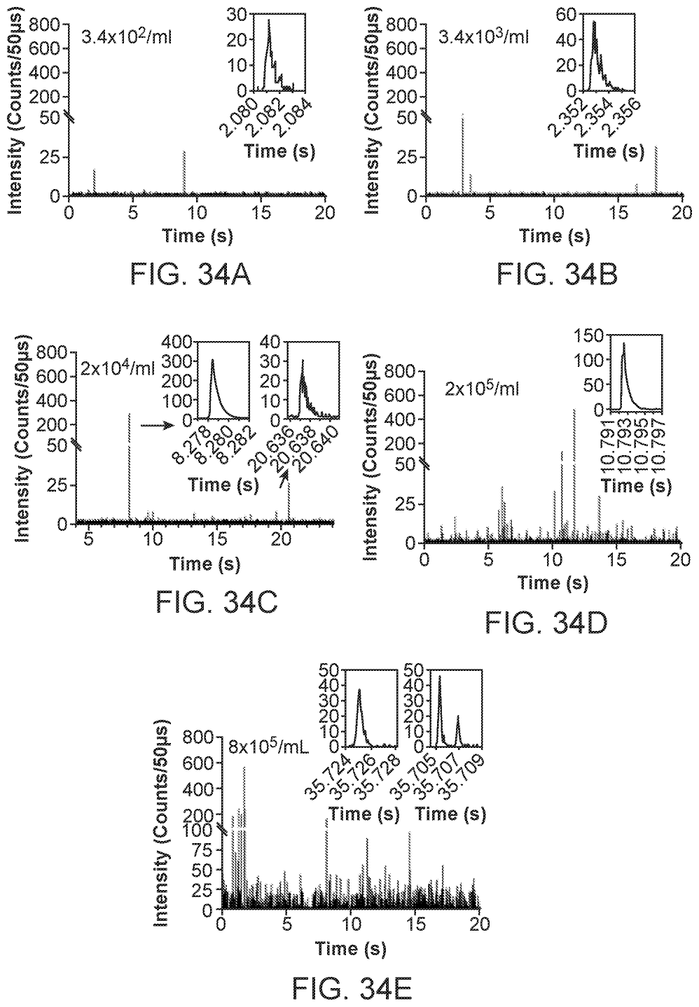

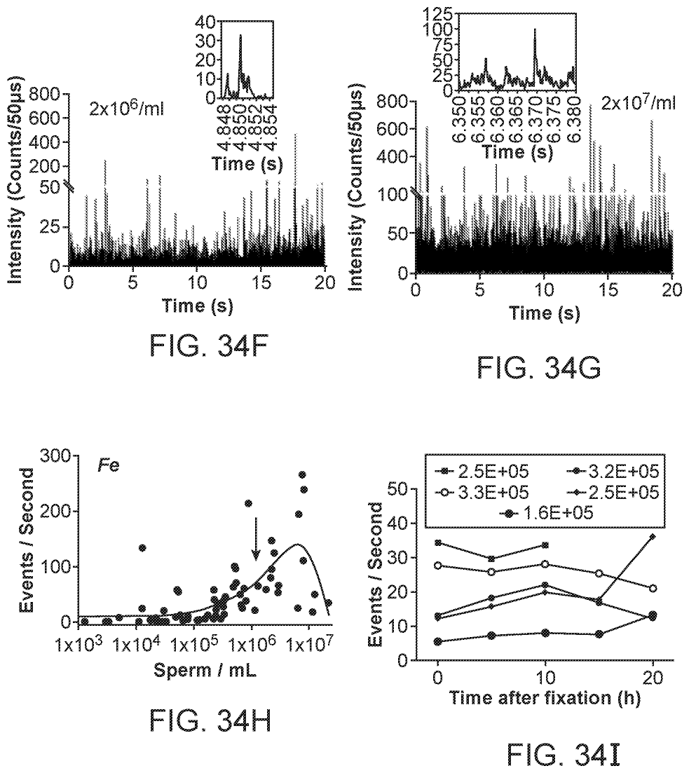

In order to obtain sensitive and accurate signals of single sperm cells by ICP-MS, the number density of sperm was controlled, and only one cell was transported to the plasma at any given time, which is determined by dwell time. In FIG. 34A-34I, it shows that the signal profile of element Fe increased as the cell density loaded into ICP-MS for measurement was increased. The cell density increased linearly with the cell events ranging from 3×10⁴ per mL to ˜2×10⁶ per mL, while the cell events decreased beyond a cell density higher than ˜3×10⁶ per mL, without wishing to be bound by the theory, reflecting the overlap of cell signals. In order to avoid false positive signals, those with cell densities higher than 1×10⁶ per mL were excluded from statistical analysis. No obvious change of cell events over 20 h after fixation of sperm cells was observed and the sample was stored in pure water before measurement.

### Example 3: Analysis of Dynamic Elementomic Profile During Capacitation of Mouse Spermatozoa Using Single-Cell ICP-MS

Analysis of metal Ion flux during capacitation of mouse spermatozoa using single-cell ICP-MS: Currently, the clinical analyses of male infertility mainly rely on semen analysis and sperm parameters. However, the high diagnostic failure rate indicates that the current evaluation methods are still insufficient, and a new approach for evaluating sperm function still needs to be developed. With increasing evidence showing the role of biometals in reproductive biology, the importance of metal ion homeostasis in sperm function and male fertility is emergent. To determine the changes of metals in single sperm cells during fertilization activities, single-cell inductively coupled plasma mass spectrometry (sc-ICP-MS) technology was employed to measure the metal concentrations of capacitating sperm. This study used a male sterile calcium pump PMCA4 knockout mouse model with abnormal calcium regulation during the fertilization events. Consistently, the resulted showed an abnormal dynamic calcium profile in the PMCA4-KO sperm undergoing capacitation. Overall, the study demonstrates that sc-ICP-MS can be applied for sperm functional analysis.

### Materials and Methods

Materials and reagents: All reagents used were analytical grade or at the highest quality available. Sodium chloride, potassium chloride, and potassium dihydrogen phosphate were purchased from Sinopharm Company (Shanghai, China). Nitric acid (65%), calcium chloride, magnesium sulfate heptahydrate, glucose, HEPES, sodium lactate, sodium pyruvate, sodium bicarbonate and phenol red were all purchased from Sigma-Aldrich Company (St. Louis, MO USA), and BSA was purchased from BBI Life Science Company (Shanghai, China). Single cell ICP-MS spherical 60 nm gold nanoparticles element standard was supplied by sc-ICP-MS analytical equipment manufacturer (PerlinElemer, Shanghai, China). Ultrapure water (18.2 MΩ) was used. Standard solutions of the desired concentrations were prepared daily using a stepwise dilution of the stock solution.

Animals: Pmca4 knockout (KO) mouse model was obtained from Delaware Biotechnology Institute, University of Delaware, USA and housed in Shanghai Research Center for Model Organisms. All animal experiments were performed in accordance with the guidelines on the use of laboratory animals established by the Animal Ethics Committee of ShanghaiTech University. Since male Pmca4^{-/-} mice were sterile, these mice were produced using heterozygous pairings. Pmca4^{+/+} (wild-type, WT) and Pmca4^{-/-} (knockout, KO) C57BL/6 mice, 8-12 weeks old, were used in this work. Forward primer (5′-CTGTGGGAACCCCGTTGGTCTCTTTC-3′) and reverse primer (5′-GCACCCAGGCGATGGATGGCAAAGCT-3′) were used for identifying the genotype of Pmca4 KO mice using PCR method, as reported previously (see, for example, Okunade et al., 2004, J Biol Chem 279, 33742-33750).

Sperm capacitation medium: HEPES buffer medium was used throughout the study for Pmca4 KO mouse sperm preparation and capacitation. The composition of the HEPE-buffered medium was 95 mM NaCl; 5 mM KCl; 1.7 mM CaCl₂; 1.2 mM MgSO₄·7H₂O; 1.2 mM KH₂PO₄; 20 mM sodium lactate (60%); 0.27 mM sodium pyruvate; 25 mM NaHCO₃; 50 mM glucose; 3 mg/mL BSA; 20 mM HEPES and 0.02 mg/mL phenol red. All the solutions were first prepared without HEPES, BSA, NaHCO₃ or CaCl₂. The HEPES buffer was sterilized by passage through a 0.22 μm filter before using and warmed on a 37° C. metal block.

Preparation of sperm samples: The mice were sacrificed by anaesthetization with sodium pentobarbital and the epididymides were quickly removed. Then the spermatozoa were obtained for capacitation assay as previously reported (see, for example, Zi et al., PLoS Genet, 2015, 11, e1005485; and Ma et al., iScience, 2019, 14, 210-225). Briefly, the caudal regions of epididymides were cut twice before being gently shaken into the HEPES buffered capacitation medium, which allowed the sperm to flow out of the epididymal tubules, and the epididymides were removed after shaking. The HEPES-buffered capacitation medium containing the sperm was placed in a 37° C. metal-bath for 5 min and assigned the time as 0 h. At the indicated time points, the spermatozoa were immediately aspirated with a 1-ml injection infusion pumping (Harvard Apparatus) into the aspiration chamber for single-cell ICP-MS analysis.

Instrumentation: For single-cell ICP-MS experiments, used were the quadrupole-based Perkin-Elmer NexION 2000 ICP-MS equipped with the single cell analysis module and the Asperon™ single cell spray chamber under the operating conditions listed in Table 4. Before analysis, the capacitated sperm was collected at the indicated time points through centrifugation firstly, and the capacitated buffer was removed. By adding appropriate volume of HEPES-buffered capacitation solution, the sperm was resuspended and diluted to 10⁵/mL using hemacytometer before loading for the ICP-MS analysis. 3% of nitric acid followed with deionized water was used to wash the aspiration chamber between samples.

TABLE 4
Parameters for Single Cell Analysis Using NexION 2000 ICP-MS.
Instrumental Parameters

| Sample Flow Rate | 0.04 | mL/min |
|---|---|---|
| Dwell Time | 50 | μs |
| Scan Time | 60 | s |
| Transport Efficiency | 6.95% | |
| Threshold | 10.26 | Counts |

Statistics: All experiments were repeated at least three times using independent WT and Pmca4 KO mouse sperm samples. Data are presented as means SD. Two-way ANOVA was performed to compare the differences amongst multiple comparisons. P-values of 0.05 or below were considered significant. Skewness and kurtosis parameters of frequency distribution versus sperm mass populations were calculated using Prism software (GraphPad).

### Results and Discussion

### Optimization of Experimental Conditions

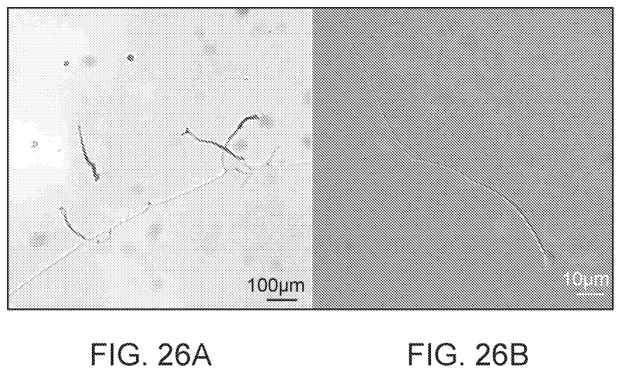

For sc-ICP-MS analysis, it is important to optimize the conditions under which cells maintain their intact cell morphology and remain in a monodispersed status, thus each ICP-MS spike corresponds to a single cellular event. Light microscopic examination showed that the sperm cells maintained their morphological integrity and were monodispersed after aspirated into the spay chamber (FIG. 26), before loading into the ICP-MS plasma chamber for elemental detection. Since sperm cells were bathed in the capacitation medium which was rich with sodium and its extracellular-to-intracellular concentration ratio was far too high for single cell analysis, whereas the interference of potassium ions was severe without other scavenging gas in the reaction chamber, and thus the contents of these two ions were then excluded from analysis in this study.

### Calcium Spectral Analysis Indicates Calcium Overload in Single Pmca4 KO Sperm Cells

In sperm, the capacitation process leads to an elevated intracellular Ca²⁺ levels through influx from the external environment. Following capacitation homeostasis in the cytosol is maintained by calcium efflux via the Pmca4 pump.

Therefore, Pmca4 deficiency leads to elevated resting intracellular Ca²⁺ concentration and resulting impaired sperm motility (see, for example, Navarrete et al., J Cell Physiol, 2015, 230, 1758-1769; and Schuh et al., 2004, J Biol Chem 279, 28220-28226). The results showed that the average resting calcium content in single Pmca4 KO sperm cells was slightly higher than that in WT cells. Prior to capacitation stimulation, the average contents at time zero was found to be 2881+256 attograms in single WT sperm and 3225+221 attograms in Pmca4 KO sperm, and were slightly decreased to 2556+212 attograms in WT sperm but increased to 3903±1375 attograms in Pmca4 KO sperm after in the capacitation physiological condition for 2 h (FIG. 27A). The overall calcium content in Pmca4 KO sperm was significantly increasingly elevated compared to the WT controls (P<0.05), especially after two hours of capacitation, although the increase was not significant at initial time points (FIG. 27A). The frequency distribution of calcium mass in single cells was altered in Pmca4 KO sperm compared to control WT sperm as capacitation progressed towards 2 h (FIGS. 27B-27F). The results showed that the data distribution at time zero basically showed normal distribution for the frequency distribution measurements of calcium content in single Pmca4 KO sperm cells compared to WT controls. Thus, the increased calcium content in Pmca4 KO sperm cells is consistent with the notion that Ca²⁺ ions are not extruded and some sperm cells accumulate more Ca²⁺ than others. Interestingly, the skewness and kurtosis parameters were significantly (P<0.05) increased at 2 h capacitation (FIGS. 27G-27H). Skewness and kurtosis, respectively, are the measures of symmetry of a frequency distribution and degree of tailedness in it. In other words, skewness measures the relative size of the two tails whereas kurtosis measures the combined sizes of the two tails of a frequency distribution. Thus, the significant positively alterations of the skewness and kurtosis in Pmca4 KO sperm cells is consistent with the failure of Ca²⁺ ion extrusion via the Pmca4 pump, resulting in some sperm cells accumulating more Ca²⁺ than others. The range of Ca mean mass content (in attogram) in single live sperm cells changed dynamically at different time points under the capacitation conditions, as analyzed by sc-ICP-MS in the same single sperm population of Pmca4 KO and WT mice, especially at the initial zero time point and the 1.5 h or 2 h under capacitation conditions (FIG. 27I). These results are consistent with the observation of increased Ca²⁺ in capacitated sperm cells in Pmca4 KO sperm, evidencing that single-cell ICP-MS technology can determine the calcium contents of sperm in physiological and pathological conditions.

### Altered Elementomic Dynamics in Single Sperm Cells of Pmca4 KO Mice During Capacitation Process

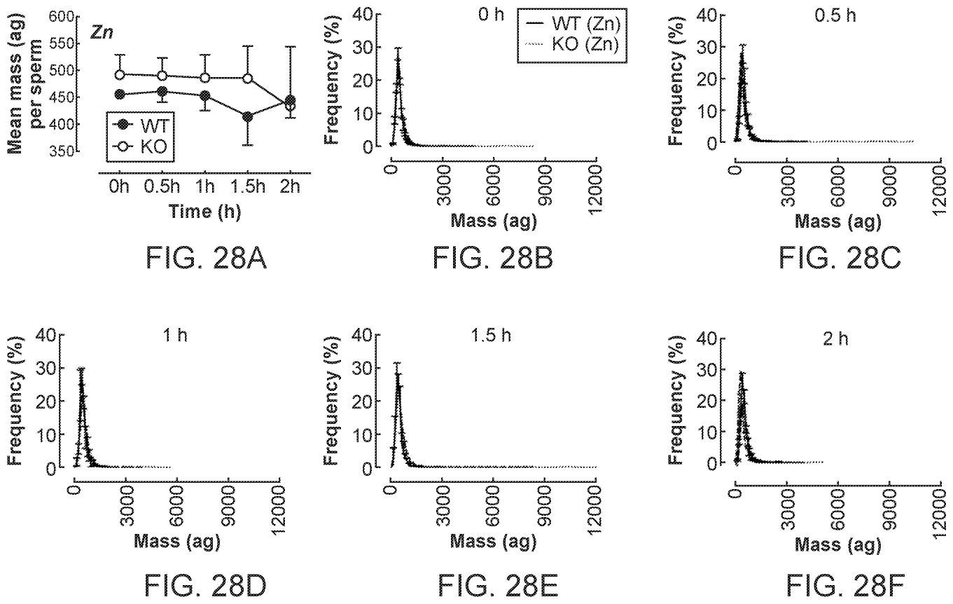

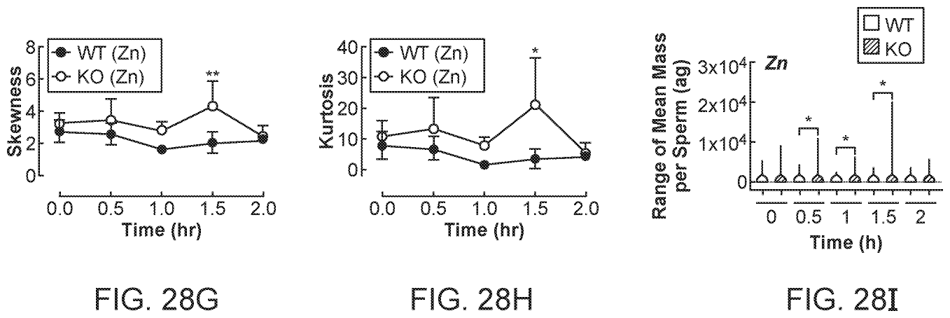

Although analyses of the mean masses and the frequency distributions of the mean mass of the elements of Zn, Fe, Cu, Mn, and Se in the single sperm of Pmca4 KO sperm cells showed no obvious differences compared to WT sperm during capacitation (FIGS. 28A-28F; 29A-29F; 30A-30F; 31A-31F; 32A-32F); however, the range of the mean mass content of these elements showed obvious altered dynamics in the single live sperm cells of Pmca4 KO mice compared to the WT sperm under the capacitation conditions, and the changes of the dynamics were especially prominent for Fe, moderate for Zn, and mild for Cu, Mn, Se elements (FIGS. 28I, 29I, 30I, 31I and 32I).

Altered Skewness and Kurtosis Parameters of Dynamic Frequency Patterns of Zinc and Manganese Ions in Single Pmca4 KO Sperm Cells

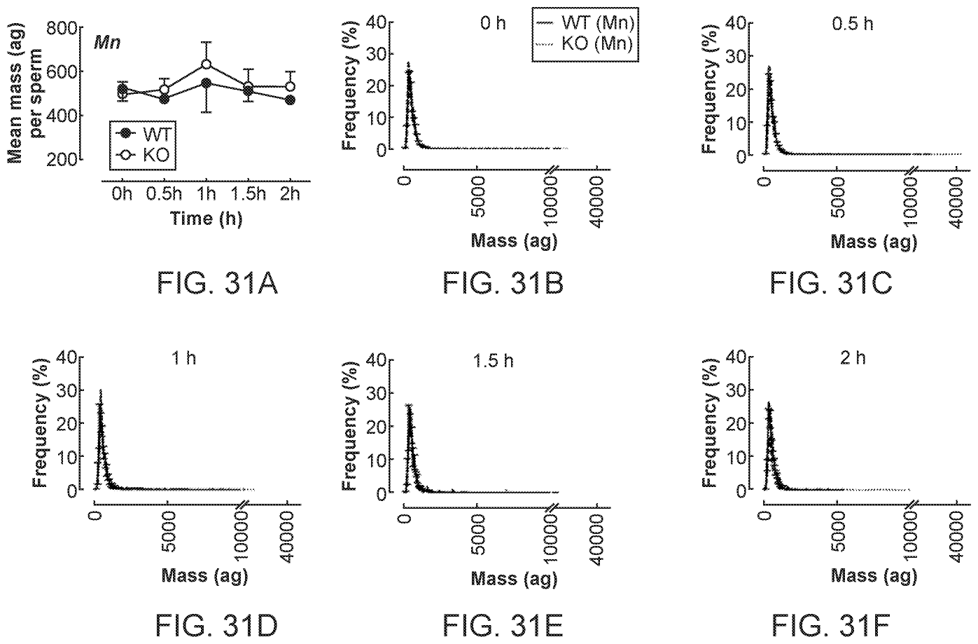

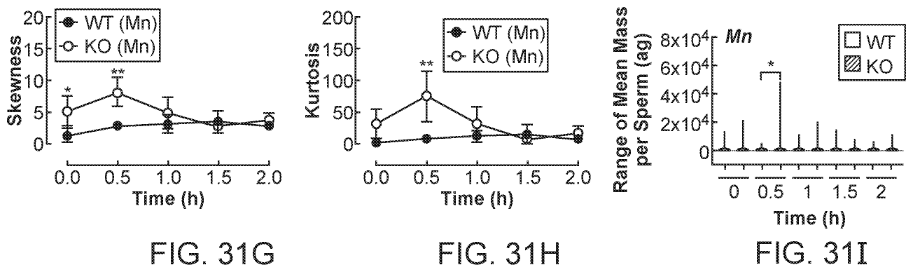

It has been reported that exogenous Zinc (Zn) leads to disrupted calcium homeostasis and cell death and elevated ROS level (see, for example, Guo et al., Arch Biochem Biophys, 2014, 560, 44-51). In addition, a high level of manganese has been reported to have a harmful effect on human sperm viability and motility (see, for example, Li et al., 2012b), whereas trace amounts of manganese have been reported to provide antioxidant protection for sperm cryopreservation (see, for example, Cheema et al., Oxid Med Cell Longev, 2009, 2, 152-159). In this study, despite the measured mass of Zn or manganese (Mn) in single sperm cells of Pmca4 KO mice were insignificant versus WT mice, however, the skewness and kurtosis parameters for the frequency distribution of Zn and Mn ions showed significant variation (P<0.05) during the course of capacitation (FIG. 28 and FIG. 31).

These results evidence that accumulated intracellular Ca²⁺ might alter the Zn- or Mn-associated redox status dynamically in Pmca4 KO sperm. Thus, experiments are under investigation to determine if this correlates with specific clinical idiopathic male infertile cases.

Characteristic Dynamic Patterns of Metallomics in Single Sperm Cells

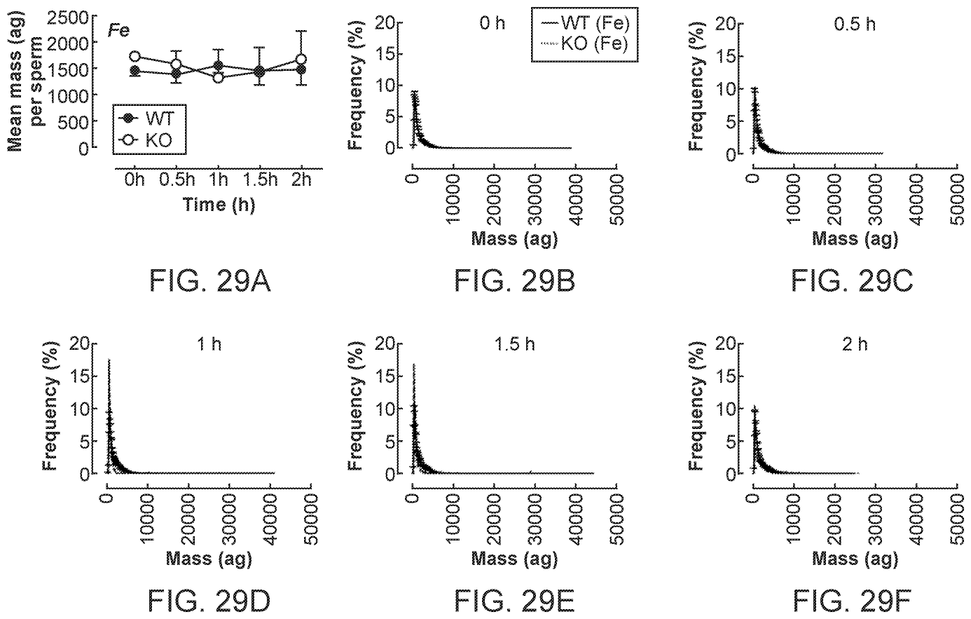

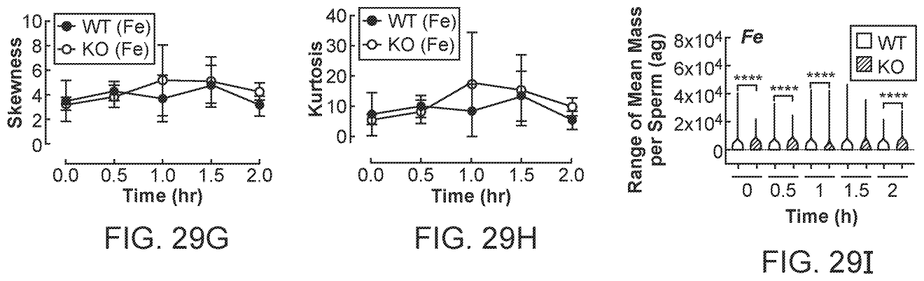

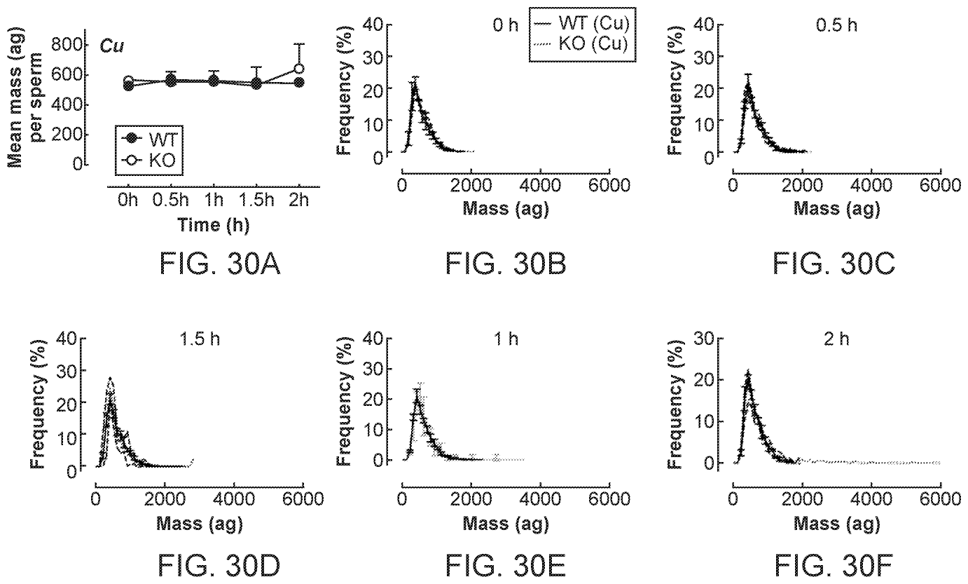

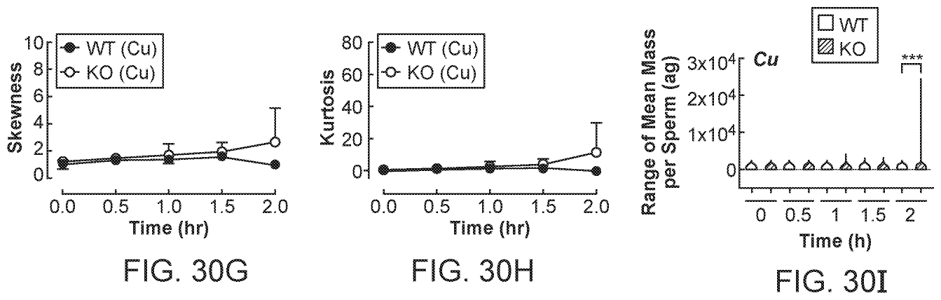

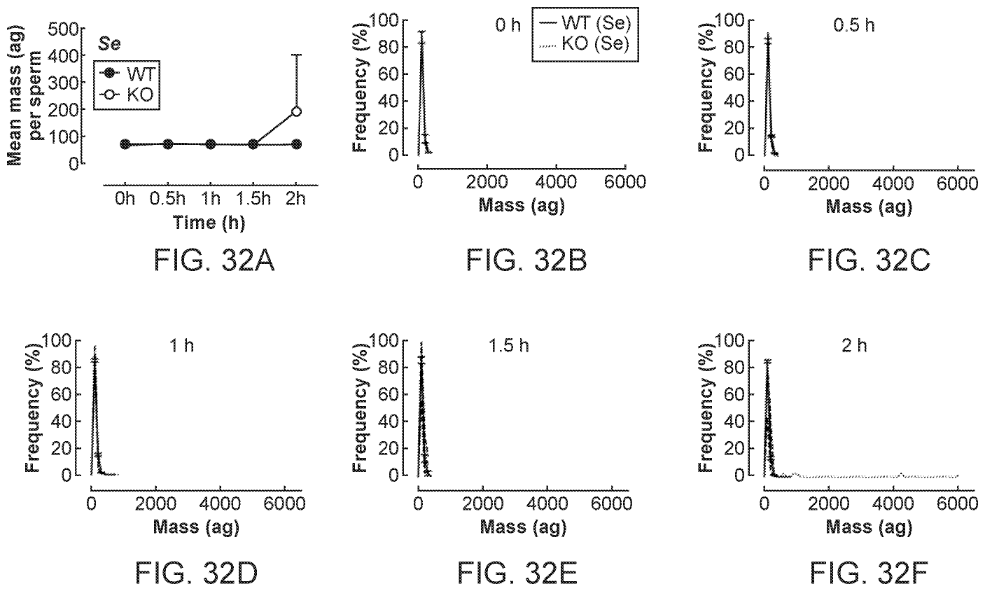

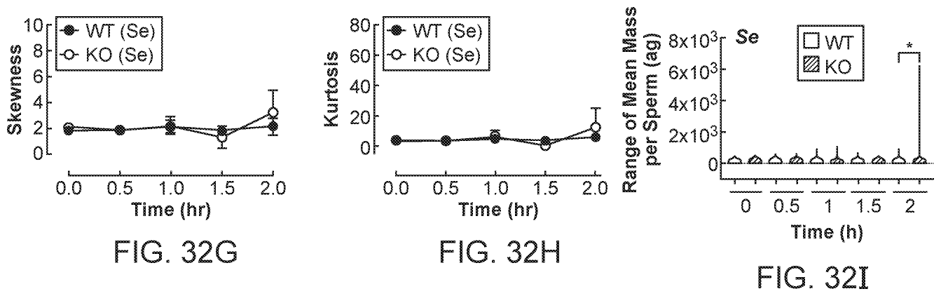

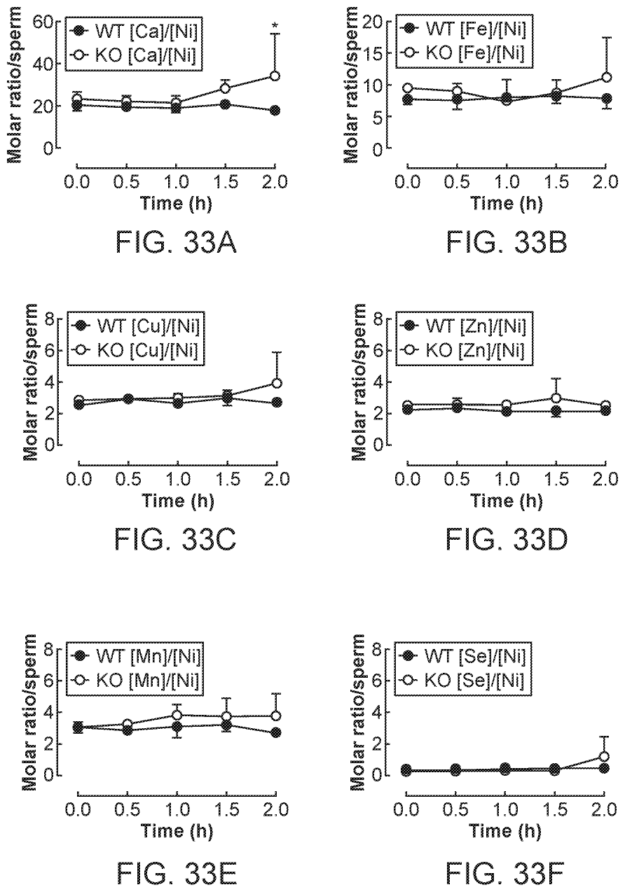

In order to elaborate the elementomics characteristics of the Pmca4 KO sperm cells, in addition to the measured and analyzed the mean masses and dynamic patterns of macro elements and rare trace elements in the same sperm samples, including calcium (FIG. 27), zinc (FIG. 28), iron (FIG. 29), copper (FIG. 30), manganese (FIG. 31), selenium (FIG. 32) ions under the sperm capacitation process, and as an attempt to further elaborate the elementomic characteristics of specific elements in the single sperm cells, the ratio of molar content of each element in single sperm cells against nickel element of each sample was determined and demonstrated in FIG. 33A-33F. We used nickel element mean molar mass as the dividend because its content was relatively low and stable and remained unchanged during the capacitation process. Results showed that the molar ratio of calcium concentration versus nickel concentration in single sperm was significantly different between the Pmca4 KO and WT groups (P<0.05) after 2 h of capacitation. These results evidence that ratiometric analysis of the elemental content of the same samples can further reveal the significance of the elementomics characteristics in single cells.

### CONCLUSIONS

This study represents an initial step toward the application of sc-ICP-MS in elementomics analysis of single sperm cells. The work demonstrates the feasibility of sc-ICP-MS to become a commonly used analytical tool for the assessment of calcium and other essential metal contents of single sperm and the dynamic elemental patterns under physiological processes, such as sperm capacitation, and thereby the assessment of sperm functional quality, and eventually male fertility of individuals. This method is rapid and simultaneously characterizes various elements and involves a relatively simple procedure. However, maintaining the accuracy of pre-test sperm integrity prior experimental procedure and post-experimental algorithmic analysis are the focus and challengers of the application of this technology in clinics. Nevertheless, the study herein shows the potential application of sc-ICP-MS to the clinics for the evaluation of human sperm functional quality, providing a multi-angle reference for clinical diagnosis.

While certain embodiments have been illustrated and described, a person with ordinary skill in the art, after reading the foregoing specification, can effect changes, substitutions of equivalents and other types of alterations to the methods of the present technology as set forth herein. Each aspect and embodiment described above can also have included or incorporated therewith such variations or aspects as disclosed in regard to any or all of the other aspects and embodiments.

The present technology is also not to be limited in terms of the particular aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to particular methods, reagents, compounds, compositions, labeled compounds or biological systems, which can, of course, vary. It is also to be understood that the terminology used herein is for the purpose of describing particular aspects only, and is not intended to be limiting. Thus, it is intended that the specification be considered as exemplary only with the breadth, scope and spirit of the present technology indicated only by the appended claims, definitions therein and any equivalents thereof.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

The present technology may include, but is not limited to, the features and combinations of features recited in the following lettered paragraphs, it being understood that the following paragraphs should not be interpreted as limiting the scope of the claims as appended hereto or mandating that all such features must necessarily be included in such claims:

A. A method for detecting multiple trace elements in trace cells, characterized in that it comprises the following steps:
Step 1: obtaining somatic cells, centrifuging the cells, removing the supernatant, suspending the centrifuged cells with a diluent to a detection concentration of lower than 3×10⁶/ml, and loading the suspended cells into an sc-ICP-MS instrument for detection;
Step 2: performing the detection method using a conventional sc-ICP-MS method: aspirating the cell suspension using a single-cell nebulizer into droplets in a single-cell spray chamber, wherein each of the droplets comprises a single cell, and analyzing multiple trace elements at the single cell level using a fast-analyzing digitizing software module;
Step 3: detecting a series of standard solutions having known concentrations of the elements using the same procedure in step 2, and plotting standard curves.
Step 4: determining the unique elemental ICP-MS signal characteristics of specific cell types using a method comprising evaluating the characteristics of sc-ICP-MS signal dynamics of specific elements of specific cells, selected from dwell time, peak time of a single signal spike, the time ratio of pre- or post-peak of the spike in relation to the dwell time, dynamic kinetic constant or area of pre-peak of the spike, dynamic constant or area of post-peak of the spike, etc;
Step 5: assessing the functional quality of the cells.
   B. A method for evaluating functional quality of a cell, characterized in that it evaluates the dynamic kinetic characteristics of sc-ICP-MS signal spikes of specific elements in specific cells, selected from dwell time, peak time of a single signal spike, ratio of the peak time to the dwell time, dynamic kinetic constant or area of pre-peak of the spike, or dynamic constant or area of post-peak of the spike.
   C. Use of the method for detecting multiple trace elements in trace cells according to Paragraph A in evaluating human sperm cells.
   D. Use of the method for detecting multiple trace elements in trace cells according to Paragraph A in evaluating various morphologies and types of cells, selected from all somatic cells, gamete cells, prokaryotic cells of any genus, or isolable cells in semen, reproductive tract fluid, follicular fluid, blood, urine, saliva or stool.
   E. Use of the method for evaluating functional quality of the cell according to Paragraph B in evaluating human sperm cells.
   F. Use of the method for evaluating functional quality of the cell according to Paragraph B in evaluating various morphologies and types of cells, selected from all somatic cells, gamete cells, prokaryotic cells, or isolable cells in semen, reproductive tract fluid, follicular fluid, blood, urine, saliva or stool.
   G. A method for detecting multiple trace elements in human sperm cells, characterized in that it comprises the following steps:
Step 1: sample preparation: liquefying fresh human semen according to the conventional semen preparation method, centrifuging the liquefied semen, separating seminal plasma and sperm, re-suspending the sperm in 4% PFA, fixing the sperm in 4% PFA for 15 minutes, removing the fixing solution by washing, and performing the following steps using the sperm or storing the sperm at four degree Celcius for later use;
Step 2: diluting the sperm with a diluent to a detection concentration of lower than 3×10⁶/ml;
Step 3: loading samples into an sc-ICP-MS instrument for detection: installing a specific single-cell nebulizer and spray chamber, tuning with a tuning solution (2% v/v nitric acid containing 10 μg/L Li, Be, Mg, Fe, In, Ce, Pb and U) to determine appropriate instrument detection parameters, using gold particle standards to determine the single cell transmission efficiency which is generally in the range of 40%-60%, preparing standard solutions having different concentrations of different elements, plotting standard curves, wherein all standard solutions are prepared in ultrapure water, wherein in order to eliminate polyatomic interference and to obtain a high signal-to-noise ratio, measurements are performed in the dynamic reaction cell (DRC) mode, using ammonia as the reaction gas for the detection of K, Ca, Cr, and Fe in single cells, or using oxygen as the reaction gas for the detection of As and Se in single cells by detecting the oxidation reaction products of AsO and SeO as the analysis species, wherein other elements are measured in standard mode, wherein the sample spike signals are sampled for 50 s with a dwell time of 50 μs (i.e. digitizing frequency at 50 MHz), and wherein the total sample volume consumed by detecting 18 elements is about 400 μL.
Step 4: determining the unique elemental ICP-MS signal characteristics of specific cell types using a method comprising evaluating the characteristics of sc-ICP-MS signal dynamics of specific elements of specific cells, selected from dwell time, peak time of a single signal spike, the time ratio of pre- or post-peak of the spike in relation to the dwell time, dynamic kinetic constant or area of pre-peak of the spike, and dynamic constant or area of post-peak of the spike;
Step 5: evaluating functional quality of the cell

Other aspects are set forth within the following claims.

1. A method for detecting infertile spermatozoa in a sample obtained from a subject, the method comprising: detecting a concentration of at least one metal in the sample that falls outside of a predetermined range using single cell inductively coupled plasma mass spectrometry (sc-ICP-MS).

2. The method of claim 1, wherein the at least one metal is selected from the group consisting of sodium (Na), potassium (K), calcium (Ca), magnesium (Mg), zinc (Zn), iron (Fe), copper (Cu), selenium (Se), cobalt (Co), chromium (Cr), cadmium (Cd), manganese (Mn), arsenic (As), mercury (Hg), lead (Pb), silver (Ag), aluminium (Al), and nickel (Ni).

3. The method of claim 1, wherein the sample is diluted with a buffer prior to the detecting step.

4. The method of claim 1, wherein the predetermined range corresponds to metal concentrations detected in spermatozoa from a population of fertile subjects.

5. The method of claim 1, wherein the spermatozoa are capacitated.

6. The method of claim 1, wherein the spermatozoa are not capacitated.

7. The method of claim 1,
wherein the predetermined range of the Na concentration is between about 5 attogram (ag) to about 50,000 ag for spermatozoa that are not capacitated and between about 25 ag to about 50,000 ag for spermatozoa that are capacitated;
wherein the predetermined range of the K concentration is between about 50 ag to about 50,000 ag for spermatozoa that are not capacitated and between about 280 ag to about 50,000 ag for spermatozoa that are capacitated;
wherein the predetermined range of the Ca concentration is between about 200 ag to about 50,000 ag for spermatozoa that are not capacitated and between about 700 ag to about 20,500 ag for spermatozoa that are capacitated;
wherein the predetermined range of the Mg concentration is between about 8 ag to about 50000 ag for spermatozoa that are not capacitated and between about 75 ag to about 15,100 ag for spermatozoa that are capacitated;
wherein the predetermined range of the Zn concentration is between about 5 ag to about 50,000 ag for spermatozoa that are not capacitated and between about 20 ag to about 50,000 ag for spermatozoa that are capacitated;
wherein the predetermined range of the Fe concentration is between about 5 ag to about 50,000 ag for spermatozoa that are not capacitated and between about 13 ag to about 50,000 ag for spermatozoa that are capacitated;
wherein the predetermined range of the Al concentration is between about 3 ag to about 50,000 ag for spermatozoa that are not capacitated and between about 6 ag to about 46700 ag for spermatozoa that are capacitated;
wherein the predetermined range of the Se concentration is between about 59 ag to about 50,000 ag for spermatozoa that are not capacitated and between about 62 ag to about 45,810 ag for spermatozoa that are capacitated;
wherein the predetermined range of the Co concentration is between about 3 ag to about 3,700 ag for spermatozoa that are not capacitated and between about 9 ag to about 20,200 ag for spermatozoa that are capacitated;
wherein the predetermined range of the Cu concentration is between about 9 ng to about 50,000 ag for spermatozoa that are not capacitated and between about 9 ng to about 37,590 ag for spermatozoa that are capacitated;
wherein the predetermined range of the Cr concentration is between about 4 ng to about 50,000 ag for spermatozoa that are not capacitated and between about 5 ag to about 46,700 ag for spermatozoa that are capacitated; and
wherein the predetermined range of the Mn concentration is between about 2 ag to about 50,000 ag for spermatozoa that are not capacitated and between about 7 ag to about 32,610 ag for spermatozoa that are capacitated.

8. A method for detecting infertile spermatozoa in a sample obtained from a subject, comprising: detecting a dynamic or kinetic parameter of a signal spike of at least one metal selected from the group of sodium (Na), potassium (K), calcium (Ca), magnesium (Mg), zinc (Zn), iron (Fe), copper (Cu), selenium (Se), cobalt (Co), chromium (Cr), cadmium (Cd), manganese (Mn), arsenic (As), mercury (Hg), lead (Pb), silver (Ag), aluminium (Al), and nickel (Ni) in the sample that falls outside of a predetermined range by single cell inductively coupled plasma mass spectrometry (sc-ICP-MS).

9. The method of claim 8, wherein the predetermined range corresponds to dynamic or kinetic parameters detected in spermatozoa from a population of fertile subjects.

10. The method of claim 9, wherein the dynamic or kinetic parameter of the spike are selected from: dwell time, pre-peak dwell time, post-peak dwell time, peak time, ratio between the peak time and the dwell time, raising tau constant before the peak, dynamic area before the peak, tailing tau constant after the peak, dynamic area after the peak, or any combination thereof.

11. The method of claim 10, wherein the dynamic or kinetic parameter of the spike comprises:
(a) a dwell time of the Fe spike between about 1.4 to about 7.9 ms for spermatozoa that are not capacitated and a dwell time of the Fe spike between about 1.5 to about 6.7 ms for spermatozoa that are capacitated;
(b) a tailing tau constant after the peak of the Fe spike about between about 0.18 to about 0.81 ms for spermatozoa that are not capacitated and a tailing tau constant after the peak of the Fe spike between about 0.18 to about 0.90 ms for spermatozoa that are capacitated;
(c) a raising tau constant before the peak of the Fe spike at about −0.35 ms or lower for spermatozoa that are not capacitated and a raising tau constant before the peak of the Fe spike at about −0.80 ms for spermatozoa that are capacitated;
(d) a dwell time of the Cu spike at about 1.5 ms or shorter for spermatozoa that are not capacitated and that are capacitated;
(e) a raising tau constant before the peak of the Cu spike at about −0.2 ms or lower for spermatozoa that are not capacitated and a raising tau constant before the peak of the Cu spike at about −0.6 ms or lower for spermatozoa that are capacitated;
(f) a tailing tau constant after the peak of the Cu spike at about 0.15 ms or lower for spermatozoa that are not capacitated and a tailing tau constant after the peak of the Cu spike at about 0.2 ms or lower for spermatozoa that are capacitated;
(g) a dwell time of the Zn spike at about 2.1 ms or shorter for spermatozoa that are not capacitated and a dwell time of the Zn spike at about 1.2 ms or shorter for spermatozoa that are capacitated;
(h) a raising tau constant before the peak of the Zn spike at about −0.25 ms or lower for spermatozoa that are not capacitated and a raising tau constant before the peak of the Zn spike at about −0.20 ms or lower for spermatozoa that are capacitated;
(i) a tailing tau constant after the peak of the Zn spike at about 1.15 ms or lower for spermatozoa that are not capacitated and a tailing tau constant after the peak of the Zn spike at about 0.25 ms or lower for, spermatozoa that are capacitated;
(j) a dwell time of the Cr spike at about 3.25 ms or shorter for spermatozoa that are not capacitated and a raising tau constant before the peak of the Cr spike at about −0.45 ms or lower for spermatozoa that are not capacitated;
(k) a tailing tau constant after the peak of the Cr spike between about 0.2 to about 0.5 ms for spermatozoa that are not capacitated;
(l) a dwell time of the Se spike at about 1.5 ms or shorter for spermatozoa that are not capacitated;
(m) a raising tau constant before the peak of the Se spike at about −0.15 ms or lower for spermatozoa that are not capacitated; and
(n) a tailing tau constant after the peak of the Se spike at about 0.25 ms or lower for spermatozoa that are not capacitated.

12. A method for detecting infertile spermatozoa in a sample, comprising:
(i) contacting a first population of the spermatozoa from the subject with a HTF (Human Tubal Fluid) buffer; and
(ii) detecting a concentration of at least one metal selected from the group of potassium (K), calcium (Ca), magnesium (Mg), mercury (Hg), silver (Ag), and aluminium (Al) in the first population of spermatozoa post the contacting step that is comparable to or lower than a concentration present in a second population of spermatozoa not contacted with a HTF buffer using single-cell inductively coupled plasma mass spectrometry (sc-ICP-MS); or
(iii) detecting a concentration of at least one metal selected from the group of sodium (Na), potassium (K), calcium (Ca), magnesium (Mg), zinc (Zn), iron (Fe), copper (Cu), selenium (Se), cobalt (Co), chromium (Cr), cadmium (Cd), manganese (Mn), arsenic (As), mercury (Hg), lead (Pb), silver (Ag), aluminium (Al), and nickel (Ni) in the first population of spermatozoa post the contacting step that is comparable to or lower than a concentration present in a second population of spermatozoa not contacted with a HTF buffer using single-cell inductively coupled plasma mass spectrometry (sc-ICP-MS); or
(iv) detecting a concentration of selenium (Se) in the first population of spermatozoa post the contacting step that is comparable to or higher than a concentration present in the second population of spermatozoa not contacted with a HTF buffer using the sc-ICP-MS; or
(v) each of (ii), (iii), and (iv).

13. The method of claim 12, wherein the sample is obtained from a subject.

14. The method of claim 13, wherein the subject has or suspect of having idiopathic infertility, asthenozoospermia, oligozoospermia, or oligoasthenozoospermia.

15. The method of claim 13, further comprising treating the subject with an infertility therapy or infertility procedure.

16. The method of claim 15, wherein the concentration of at least one metal in the spermatozoa is lower than the predetermined range and the infertility therapy comprises treatment with the at least one metal.

17. (canceled)

18. The method of claim 1, wherein the spermatozoa is diluted to a concentration of 3×10⁶ spermatozoa/ml or less prior to the detecting step.

19. The method of claim 1, wherein the spermatozoa is diluted to 10 times or more prior to the detecting step.

20. The method of claim 18, wherein the spermatozoa is centrifuged to remove the seminal plasma prior to the dilution.

21. The method of claim 1, wherein the sample comprises semen, optionally liquefied; or fixed; or capacitated; or cryopreserved; or liquefied and fixed; or liquefied and capacitated; or liquefied and cryopreserved; or fixed and capacitated; or fixed and cryopreserved; or capacitated and cryopreserved; or liquefied, fixed and capacitated; or liquefied, fixed and cryopreserved; or liquefied, capacitated and cryopreserved; or liquefied, fixed, capacitated and cryopreserved.

22.-24. (canceled)

25. A kit comprising buffer and instructions for performing the method of claim 1.

26. The method of claim 8, wherein the spermatozoa is diluted to a concentration of 3×10⁶ spermatozoa/ml or less prior to the detecting step.

27. The method of claim 8, wherein the spermatozoa is diluted to 10 times or more prior to the detecting step.

28. The method of claim 26, wherein the spermatozoa is centrifuged to remove the seminal plasma prior to the dilution.

29. The method of claim 8, wherein the sample comprises semen, optionally liquefied; or fixed; or capacitated; or cryopreserved; or liquefied and fixed; or liquefied and capacitated; or liquefied and cryopreserved; or fixed and capacitated; or fixed and cryopreserved; or capacitated and cryopreserved; or liquefied, fixed and capacitated; or liquefied, fixed and cryopreserved; or liquefied, capacitated and cryopreserved; or liquefied, fixed, capacitated and cryopreserved.

30. A kit comprising buffer and instructions for performing the method of claim 8.
